(19) United States
(12) Patent Application Publication
(10) Pub. No.: US 2018/0072945 A1
(43) Pub. Date: Mar. 15, 2018

(54) MATERIAL FOR ORGANIC ELECTROLUMINESCENT ELEMENTS, ORGANIC ELECTROLUMINESCENT ELEMENT, DISPLAY DEVICE AND LIGHTING DEVICE

(71) Applicant: Konica Minolta, Inc., Tokyo (JP)

(72) Inventors: Shinya OTSU, Koganei-shi, Tokyo (JP); Tetsuya YAMADA, Kawasaki-shi, Kanagawa (JP); Motoaki SUGINO, Akishima-shi, Tokyo (JP)

(21) Appl. No.: 15/562,731

(22) PCT Filed: Apr. 18, 2016
(86) PCT No.: PCT/JP2016/062213

§ 371 Date: Sep. 28, 2017

(30) Foreign Application Priority Data
Apr. 27, 2015 (JP) 2015-090408
Dec. 4, 2015 (JP) 2015-237481

(51) Int. Cl. C09K 11/06; H01L 51/50; H01L 51/52; H01L 27/32

(52) U.S. Cl. CPC C09K 11/06; H01L 51/5036; H01L 27/32; H01L 51/5203; H01L 51/5048

(57) ABSTRACT

To provide a material for organic EL elements, which is capable of suppressing voltage increase during driving of an organic electroluminescent element as well as decrease of initial voltage caused by easy level control and mobility improvement, and which is additionally capable of improving luminous efficiency. A material for organic EL elements according to the present invention is characterized by containing a compound having a structure represented by general formula (1),

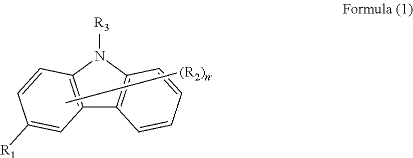

### TECHNICAL FIELD

The present invention relates to a material for an organic electroluminescent element, an organic electroluminescent element, a display device and a lighting device. In particular, the present invention relates to a material for an organic electroluminescent element enabling to suppress decrease of initial voltage and voltage increase during driving, and further to improve emission efficiency, an organic electroluminescent element, a display device and a lighting device.

### BACKGROUND

An organic electroluminescent element (hereafter, it is also called as an organic EL element) is a light emitting element having a constitution in which a light emitting layer containing a luminescent organic compound is interposed between a cathode and an anode. A hole injected from an anode and an electron injected from a cathode are recombined in the light emitting layer by applying an electric field, thus, an exciton is formed. It uses emitted light (fluorescence and phosphorescence) when the above exciton is deactivated. An organic EL element is a totally solid state element constituted by a film of an organic material having a thickness of only submicron and it enables to emit light at a voltage of several voltages to several ten voltages. Therefore, it is expected to be used for a flat display and an illumination of the next generation.

As a development of an organic EL element toward practical application, it was reported an organic EL element making use of phosphorescence emitted from an excited triplet state from Princeton University. Thereafter, there have been actively investigated materials that emit phosphorescence at room temperature.

Further, organic EL elements operated by making use of phosphorescence emission make it possible to achieve a light emitting efficiency which is theoretically larger by about four times than those of conventional organic EL elements operated by making use of fluorescence emission. Therefore, starting from material development, a layer structure and electrodes of a light emitting element for the organic EL elements have been investigated and developed all over the world.

As described above, a phosphorescence emission method has a high potential. However, the phosphorescence material is usually used as a mixed film with a so-called host compound. The reasons of this usage are mainly the following two. One of them is to employ the host compound as a dispersion agent to avoid decrease of emission efficiency caused by aggregation of the emission material. The other reason is to achieve the role of transport a charge (hole and electron) to an emission material.

Here, an electron transport and injection mechanism will be described by referring to FIG. 7.

An organic EL element material is an insulating organic molecule. Therefore, electrons and holes cannot be directly injected from an anode and a cathode into a dopant (it cannot be carried out charge injection complied with an Ohm's law.

In order to inject and transport charge into this insulating organic compound, it is required to make a thin film (thickness of 100 nm or less), and to decrease an energy barrier. That is, since the energy barrier between the anode and the light emitting layer is large, holes cannot be directly injected. Consequently, it is required to have a hole injection-transport thin layer having an intermediate energy level between the anode and the light emitting layer. Further, with respect to electrons, it is required to have an electron injection-transport thin layer. In addition, the charge will make hopping transfer as a general rule between the space of a nt conjugated portion of an organic molecule. Therefore, all materials for an organic EL element have a chemical structure containing a combination of aromatic compounds such as benzene and pyridine.

An electron is injected from the cathode to a LUMO level of an organic molecule to result in forming an anion radical. Since the anion radical is unstable, the electron is transferred to an adjacent molecule. When this process is repeated, it appears that only the electron is moved from the right side to the left side in the figure.

On the other hand, an electron is transferred from a HOMO level of an adjacent organic molecule to the anode. Namely, a hole is injected to result in forming a cation radical. This is moved from the left side to the center of the figure.

Namely, in order to achieve charge transfer-injection, it is important to have a nt conjugated portion that enables to control a HOMO level and LUMO level of an organic molecule, and to make hopping transfer.

There are two methods for controlling (deepening) a HOMO level and a LUMO level. One of them is to introduce an aromatic heterocycle containing an electron attractive N atom (for example, pyridine, pyrimidine, triazine, and quinoline). The second method is to introduce an electron attractive group. The latter method is more easily done for molecular design. By introducing it into a known material for an organic EL element, the required HOMO level and LUMO level may be easily achieved. A cyano group or a trifluoromethyl group (an electron attractive group) is often used for this purpose (refer to Patent document 1 and Patent document 2).

However, when an electron attractive group such as a cyano group or a trifluoromethyl group is introduced in an organic compound, it will be produced large intermolecular polarization (generation of plus a positive charge portion and a negative charge portion in the molecule). Thereby, it may be induced a large intermolecular charge interaction (intermolecular attraction between a positive charge portion and a negative charge portion. This will weaken an intermolecular n-n interaction that is primarily required for carrier hopping. As a result, it will be produced decrease of transfer. In particular, decrease of electron transfer is pronounced.

### PRIOR ART DOCUMENTS

### Patent Documents

Patent document 1: WO 2005/044795

Patent document 2: WO 2012/005269

### SUMMARY OF THE INVENTION

### Problems to be Solved by the Invention

The present invention has been made in view of the above-described problems and situation. An object of the present invention is to provide a material for an organic electroluminescent element capable of suppressing: decrease of the initial driving voltage caused by easy energy level control and improved mobility; and voltage increase during driving of the organic electroluminescent element. Further, the material enables to improve emission efficiency. An object of the present invention is to provide an electroluminescent element, a display device, and a lighting device.

### Means to Solve the Problems

The present inventors have made investigation into the reasons of the above-described problems in order to solve the problems. As a result, the present inventors found out the following and achieved the invention.

When a carbazole derivative that is introduced with a cyano group or a trifluoromethyl group and a condensed ring is used as a material for an organic electroluminescent element, it is possible to suppress decrease of initial voltage, and to improve emission efficiency, and further to suppress voltage increase during driving of the element.

Further, when dibenzofuran is used as a condensed ring, an intermolecular π-π interaction becomes large, and as a result, molecular motion during driving of the element is restrained and voltage increase during driving becomes small. Further, the movement of the emission dopant during driving may be restrained. This will prevent aggregation of the emission dopant during driving of the element, and the stability of exciton of the emission dopant is improved.

That is, the above-described problems of the present invention are solved by the following means.

1. An organic electroluminescent element material containing a compound having a structure represented by Formula (1).

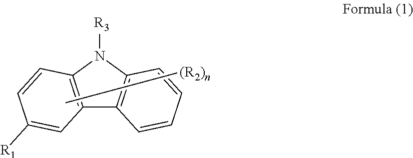

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; and n represents an integer of 0 to 7, provided that when R₂ and R₃ each independently represent an alkyl group, an aryl group, a heteroaryl group, or a fluoroalkyl group, at least one of R₂ and R₃ contains a structure represented by Formula (2).

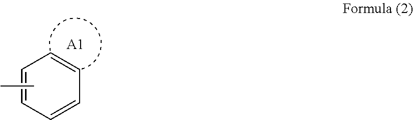

In Formula, A1 represents a 5-membered heterocycle, provided that the 5-membered heterocycle may further have a substituent, and the substituent may form a ring.

2. The organic electroluminescent element material described in the item 1,

wherein in the compound represented by Formula (1), when R₂ and R₃ each independently represent an alkyl group, an aryl group, a heteroaryl group, or a fluoroalkyl group, at least one of R₂ and R₃ contains a substituent represented by Formula (2).

3. The organic electroluminescent element material described in the item 1,

wherein R₂ and R₃ each independently represent an alkyl group, an aryl group, a heteroaryl group, or a fluoroalkyl group, at least one of R₂ and R₃ in itself represents a substituent represented by Formula (2).

4. The organic electroluminescent element material described in any one of the items 1 to 3,

wherein the compound having a structure represented by Formula (1) is a compound having a structure represented by Formula (3).

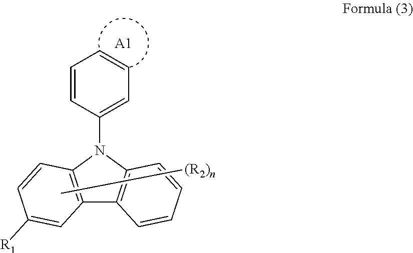

In Formula, R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and A1 represents a 5-membered heterocycle, provided that the 5-membered heterocycle may further have a substituent, and the substituent may form a ring.

5. The organic electroluminescent element material described in any one of the items 1 to 3,

wherein the compound having a structure represented by Formula (1) is a compound having a structure represented by Formula (4).

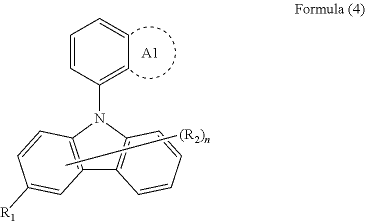

In Formula, R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and A1 represents a 5-membered heterocycle, provided that the 5-membered heterocycle may further have a substituent, and the substituent may form a ring.

6. The organic electroluminescent element material described in any one of the items 1 to 3,

wherein the compound having a structure represented by Formula (1) is a compound having a structure represented by Formula (5).

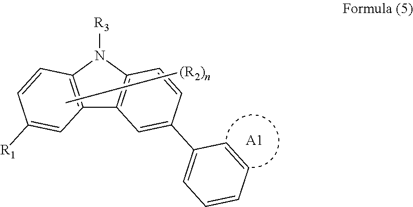

In Formula, R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; n represents an integer of 0 to 6; and A1 represents a 5-membered heterocycle, provided that the 5-membered heterocycle may further have a substituent, and the substituent may form a ring.

7. The organic electroluminescent element material described in any one of the items 1 to 3,

wherein the compound having a structure represented by Formula (1) is a compound having a structure represented by Formula (6).

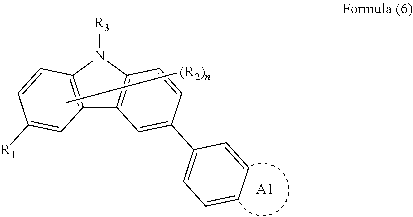

In Formula, R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; n represents an integer of 0 to 6; and A1 represents a 5-membered heterocycle, provided that the 5-membered heterocycle may further have a substituent, and the substituent may form a ring.

8. The organic electroluminescent element material described in any one of the items 1 to 7,

wherein A1 in Formula (2) represents one selected from the group consisting of: a furan ring, a thiophene ring, a pyrrole ring, an indole ring, a benzofuran ring, a benzothiophene ring, a pyrazole ring, an irmidazole ring, a triazole ring, an oxazole ring, and a thiazole ring.

9. The organic electroluminescent element material described in any one of the items 1 to 8,

wherein the compound having a structure represented by Formula (1) has an emission maximum wavelength of a 0-0 transition band in a phosphorescence spectrum to be 450 nm or less.

10. The organic electroluminescent element material described in any one of the items 1 to 9,

wherein a LUMO level of a condensed ring compound in a substituent containing a structure represented by Formula (2) is lower than a LUMO level of carbazole.

11. The organic electroluminescent element material described in the item 1,

wherein the compound having a structure represented by Formula (1) is a compound having a structure represented by Formula (7).

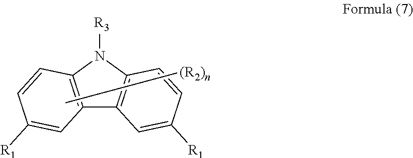

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; and n represents an integer of 0 to 6, provided that when R₂ and R₃ each independently represent an alkyl group, an aryl group, a heteroaryl group, or a fluoroalkyl group, at least one of R₂ and R₃ contains a structure represented by Formula (2).

12. The organic electroluminescent element material described in any one of the items 1 to 3,

wherein the compound having a structure represented by Formula (1) is a compound having a structure represented by Formula (8).

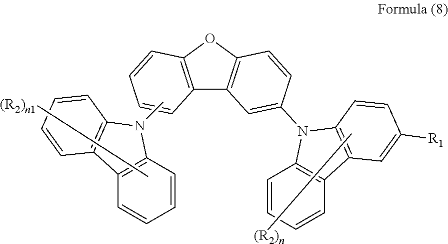

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

13. The organic electroluminescent element material described in any one of the items 1 to 3,

wherein the compound having a structure represented by Formula (1) is a compound having a structure represented by Formula (9).

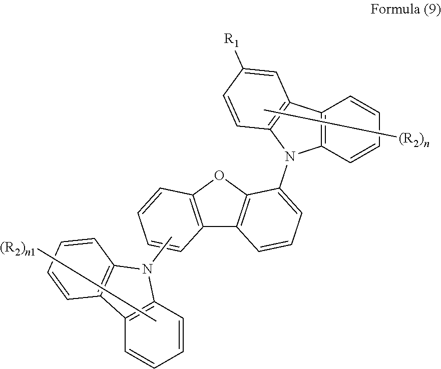

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

14. The organic electroluminescent element material described in any one of the items 1 to 3,

wherein the compound having a structure represented by Formula (1) is a compound having a structure represented by Formula (10).

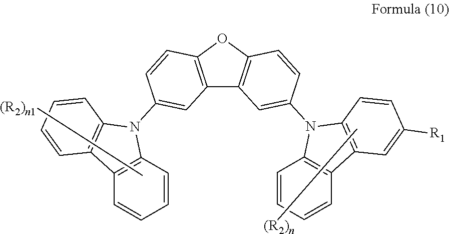

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

15. The organic electroluminescent element material described in any one of the items 1 to 3,

wherein the compound having a structure represented by Formula (1) is a compound having a structure represented by Formula (11).

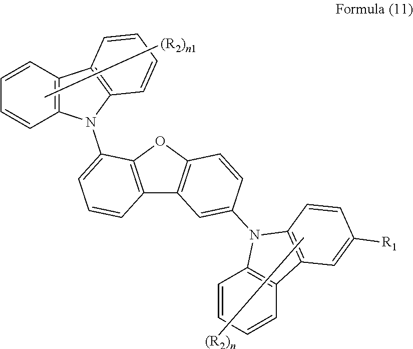

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

16. The organic electroluminescent element material described in any one of the items 1 to 3,

wherein the compound having a structure represented by Formula (1) is a compound having a structure represented by Formula (12).

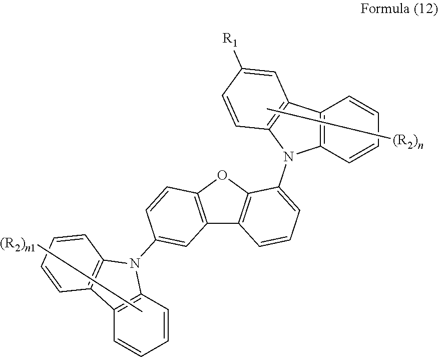

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

17. The organic electroluminescent element material described in any one of the items 1 to 3,

wherein the compound having a structure represented by Formula (1) is a compound having a structure represented by Formula (13).

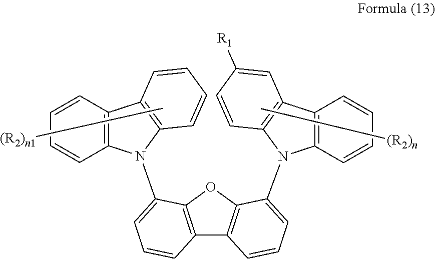

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

18. The organic electroluminescent element material described in the item 1,

wherein the compound having a structure represented by Formula (1) is a compound having a structure represented by Formula (14).

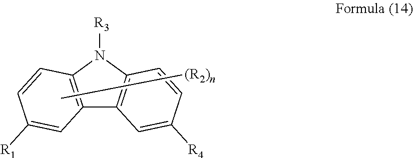

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; R₄ represents a dibenzofuran ring; and n represents an integer of 0 to 6, provided that when R₂ and R₃ each independently represent an alkyl group, an aryl group, a heteroaryl group, or a fluoroalkyl group, at least one of R₂ and R₃ contains a structure represented by Formula (2).

19. The organic electroluminescent element material described in the items 1 or 3,

wherein the compound having a structure represented by Formula (1) is a compound having a structure represented by Formula (15).

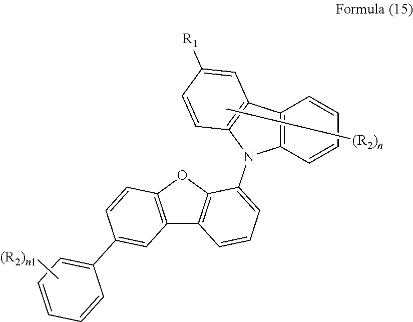

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 5.

20. The organic electroluminescent element material described in the items 1 or 3,

wherein the compound having a structure represented by Formula (1) is a compound having a structure represented by Formula (16).

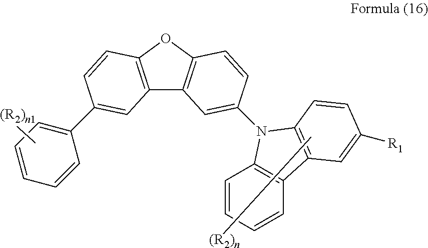

In Formula, R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 5.

21. An organic electroluminescent element containing the organic electroluminescent element material described in any one of the items 1 to 20.
22. The organic electroluminescent element described in the item 21 emitting blue light.
23. The organic electroluminescent element described in the item 21 emitting white light.
24. A display device equipped with the organic electroluminescent element described in any one of the items 21 to 23.
25. A lighting device equipped with the organic electroluminescent element described in any one of the items 21 to 23.

### Effects of the Invention

By the above-described means, it is possible to provide an organic electroluminescent element capable of suppressing: decrease of the initial driving voltage caused by easy energy level control and improved mobility; and voltage increase during driving of the organic electroluminescent element. Further, the material enables to improve emission efficiency. It is also possible to provide an electroluminescent element, a display device, and a lighting device.

An expression mechanism or an action mechanism of the effects of the present invention is not clearly identified, but it is supposed as follows.

It is possible to ensure compatibility of easy control of energy level and improved mobility by using a specific carbazole compound in at least one organic layer interposed between an anode and a cathode of an organic EL element. This carbazole compound contains an elegy level controlling group of a cyano group or a trifluoromethyl group, as well as a condensed ring having a strong π-π interaction. As a result, it is possible to suppress decrease of initial voltage, and to improve emission efficiency. Further, by an introduction of a rigid condensed ring, the glass temperature will be increased. A molecular change in the organic layer will be prevented and voltage increase during driving will be suppressed.

### BRIEF DESCRIPTION OF DRAWINGS

FIG. 1 is a schematic diagram illustrating an example of a display device including an organic EL element.

FIG. 2 is a schematic diagram of display section A

FIG. 3 is a circuit diagram of an image pixel.

FIG. 4 is a schematic diagram of a full color display device according to a passive matrix mode.

FIG. 5 is an outline diagram of a lighting device.

FIG. 6 is a schematic diagram of a lighting device.

FIG. 7 is a schematic diagram to describe an electron transfer-injection mechanism.

### EMBODIMENTS TO CARRY OUT THE INVENTION

An organic electroluminescent element material of the present invention is characterized in containing a compound having a structure represented by Formula (1).

The above-described feature is a technical feature commonly owned by or corresponding to the invention relating to each claim.

As embodiments of the present invention, it is preferable that the compound having a structure represented by Formula (1) is a compound represented by any one of Formulas (3) to (16) from the viewpoint of obtaining the effects of the present invention.

It is preferable that A1 in Formula (2) represents one selected from the group consisting of: a furan ring, a thiophene ring, a pyrrole ring, an indole ring, a benzofuran ring, a benzothiophene ring, a pyrazole ring, an imidazole ring, a triazole ring, an oxazole ring, and a thiazole ring from the viewpoint of charge transport.

It is also preferable that the compound having a structure represented by Formula (1) has an emission maximum wavelength of a 0-0 transition band in a phosphorescence spectrum to be 450 nm or less from the viewpoint of properness for blue phosphorescent host. It is also preferable that a LUMO level of a condensed ring compound in a substituent containing a structure represented by Formula (2) is lower than a LUMO level of carbazole from the viewpoint of charge transport property, in particular, from the viewpoint of electron transport property.

The organic electroluminescent element of the present invention is characterized in containing the above-described organic electroluminescent element.

It is preferable that the organic electroluminescent element of the present invention emits blue light or white light from the viewpoint of being compatible with the circumstance and achieving a versatile room illumination.

The organic electroluminescent element of the present invention is suitable to a display device or a lighting device.

The present invention and the constitution elements thereof, as well as configurations and embodiments, will be detailed in the following. In the present description, when two figures are used to indicate a range of value before and after “to”, these figures are included in the range as a lower limit value and an upper limit value.

### [Organic Electroluminescent Element Material]

### <Compound Having a Structure Represented by Formula (1)>

An organic electroluminescent element material of the present invention is characterized in containing a compound having a structure represented by Formula (1).

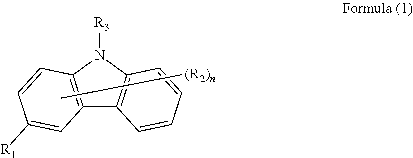

In Formula, R₁ represents a cyano group, CₘF₂ₘ₊₁, or SF₅, and m represents an integer of 1 to 18.

R₂ represents an alkyl group (for example, a methyl group, an ethyl group, a trifluorornethyl group, and an isopropyl group), an aryl group (for example, and a phenyl group), a heteroaryl group (for example, a pyridyl group, and a carbazolyl group), a halogen atom (for example, a fluorine atom), a cyano group, or a fluoroalkyl group that is substituted with any one of hydrogen atoms on carbon atoms constituting a carbazole ring. Preferably, R₂ represents an alkyl group, an aryl group, or a heteroaryl group.

R₃ represents a hydrogen atom, an alkyl group (for example, a methyl group, an ethyl group, a trifluoromethyl group, and an isopropyl group), an aryl group (for example, and a phenyl group), a heteroaryl group (for example, a pyridyl group, and a carbazolyl group), or a fluoroalkyl group. Preferably, R₃ represents an alkyl group, an aryl group, or a heteroaryl group.

“n” represents an integer of 0 to 7.

Provided that when R₂ and R₃ each independently represent an alkyl group, an aryl group, a heteroaryl group, or a fluoroalkyl group, at least one of R₂ and R₃ preferably contains a structure represented by Formula (2).

When R₂ and R₃ each independently represent an alkyl group, an aryl group, a heteroaryl group, or a fluoroalkyl group, at least one of R₂ and R₃ preferably contains a structure represented by Formula (2).

Further, it is particularly preferable that at least one of R₂ and R₃ in itself represents a substituent represented by Formula (2).

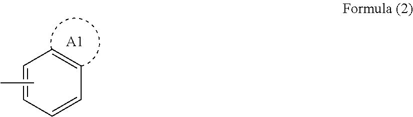

In Formula (2), A1 represents a 5-membered heterocycle, provided that the 5-membered heterocycle may further have a substituent, and the substituent may form a ring.

Examples of the 5-membered heterocycle are: a furan ring, a thiophene ring, a pyrrole ring, an indole ring, a benzofuran ring, a benzothiophene ring, a pyrazole ring, an imidazole ring, a triazole ring, an oxazole ring, and a thiazole ring.

It is particularly preferable to be a benzofuran ring, a benzothiophene ring, or an imidazole ring.

Examples of the substituent are: an alkyl group (for example, a methyl group, an ethyl group, a trifluoromethyl group, and an isopropyl group), an aryl group (for example, and a phenyl group), a heteroaryl group (for example, a pyridyl group, and a carbazolyl group), or a fluoroalkyl group. It is particularly preferable to be an alkyl group, an aryl group, or a heteroaryl group.

The compound having a structure represented by Formula (1) has preferably an emission maximum wavelength of a 0-0 transition band in a phosphorescence spectrum to be 450 nm or less, more preferably to be 440 nm or less, and still more preferably to be 430 nm or less.

A measuring method of an emission maximum wavelength of a 0-0 transition band in a phosphorescence spectrum will be described. First, a measuring method of a phosphorescence spectrum will be described.

A compound to be measured is dissolved in a mixed solvent of ethanol/methanol=4/1 (vol/vol) that has been properly degassed. After the solution is placed in a phosphorescence measuring cell, it is irradiated with excitation light at a liquid nitrogen temperature of 77 K. After irradiation with the excitation light for 100 ms, an emission spectrum is measured. Since a lifetime of phosphorescence is longer than a lifetime of fluorescence, the light remaining after 100 ms is considered to be almost phosphorescence. When a compound has a lifetime of phosphorescence of 100 ms or less, the measurement may be done with decreasing the delay time. However, when the delay time is shortened to a degree that cannot be discriminated phosphorescence and fluorescence, phosphorescence and fluorescence cannot be separated. This will cause a problem. Therefore, it is required to select a suitable delay time by which separation of phosphorescence and fluorescence is possible.

With respect to a compound that is not dissolved in the above-described solvent, it may be used an optional solvent that is capable of dissolving the compound (substantially, the solvent effect to a phosphorescence wavelength is very slight, therefore, there is no problem).

Next, a 0-0 transition band is determined. In the present invention, a 0-0 transition band is defined as an emission maximum wavelength that appears in the shortest wavelength side in the phosphorescent spectrum chart obtained with the above-described measuring method.

In many cases, a phosphorescence spectrum has a weak intensity. As a result, when the phosphorescence spectrum is expanded, in some cases, it is difficult to discriminate a noise from a peak. In these cases, an emission spectrum immediately after irradiation with excitation light (for convenience, it is called as a constant light spectrum) is expanded, and it is superimposed with an emission spectrum 100 ms after irradiation with excitation light (for convenience, it is called as a phosphorescence spectrum). From the constant light spectrum portion derived from the phosphorescence spectrum, a peak wavelength may be read and determined. By making a smoothing treatment to a the phosphorescence spectrum, a noise and a peak may be separated, and thus, a peak wavelength may be determined. As a smoothing treatment, it may be applied a smoothing method by Savitzky & Golay.

In the present invention, it is preferable that a LUMO level of a condensed ring compound in a substituent containing a structure represented by Formula (2) is lower than a LUMO level of carbazole.

Specifically, it is preferable that the LUMO level of a condensed ring compound in a substituent containing a structure represented by Formula (2) is in the range of −1.0 to −2.5 eV.

In addition, the LUMO level of carbazole is −0.6 eV.

In the present invention, the LUMO value is a value calculated with a molecular orbital calculation software Gaussian 98 (Gaussian 98, Revision A.11.4, M. J. Frisch et al., Gaussian, Inc., Pittsburgh Pa., 2002). It is defined as a value calculated by structure optimization using B3LYP/LanL2DZ as a key word (eV conversion value). The reason of efficiency of this calculation value is proved by a high correlation between the calculation value obtained with this method and the experimental value.

It is preferable that the compound represented by Formula (1) is a compound represented by any one of Formulas (3) to (16).

### <Compound Having a Structure Represented by Formula (3)>

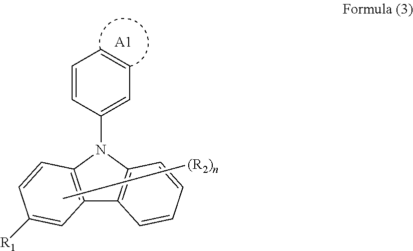

In Formula (3), R₁ represents a cyano group or CF₃.

R₂ represents an alkyl group (for example, a methyl group, an ethyl group, a trifluoromethyl group, and an isopropyl group), an aryl group (for example, and a phenyl group), a heteroaryl group (for example, a pyridyl group, and a carbazolyl group), a halogen atom (for example, a fluorine atom), a cyano group, or a fluoroalkyl group that is substituted with any one of hydrogen atoms on carbon atoms constituting a carbazole ring. Preferably, R₂ represents an alkyl group, an aryl group, or a heteroaryl group.

n represents an integer of 0 to 7.

A1 represents a 5-membered heterocycle, provided that the 5-membered heterocycle may further have a substituent, and the substituent may form a ring. As examples of the 5-membered heterocycle and the substituent, the same ones cited for Formula (1) are cited.

### <Compound Having a Structure Represented by Formula (4)>

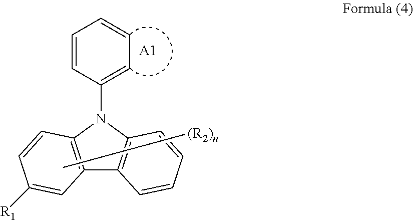

In Formula (4), R₁ represents a cyano group or CF₃.

R₂ represents an alkyl group, an aryl group, a heteroaryl group, a halogen atom, a cyano group, or a fluoroalkyl group that is substituted with any one of hydrogen atoms on carbon atoms constituting a carbazole ring. Preferably, R₂ represents an alkyl group, an aryl group, or a heteroaryl group.

n represents an integer of 0 to 7.

A1 represents a 5-membered heterocycle, provided that the 5-membered heterocycle may further have a substituent, and the substituent may form a ring. As examples of the 5-membered heterocycle and the substituent, the same ones cited for Formula (1) are cited.

### <Compound Having a Structure Represented by Formula (5)>

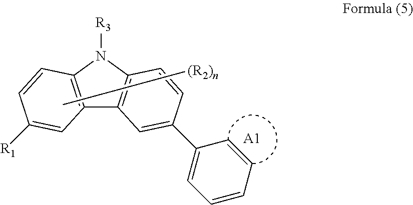

In Formula (5), R₁ represents a cyano group or CF₃.

R₂ represents an alkyl group, an aryl group, a heteroaryl group, a halogen atom, a cyano group, or a fluoroalkyl group that is substituted with any one of hydrogen atoms on carbon atoms constituting a carbazole ring. Preferably, R₂ represents an alkyl group, an aryl group, or a heteroaryl group.

R₃ represents a hydrogen atom, an alkyl group, an aryl group, a heteroaryl group, or a fluoroalkyl group. Preferably, R₃ represents an alkyl group, an aryl group, or a heteroaryl group.

n represents an integer of 0 to 6.

A1 represents a 5-membered heterocycle, provided that the 5-membered heterocycle may further have a substituent, and the substituent may form a ring. As examples of the 5-membered heterocycle and the substituent, the same ones cited for Formula (1) are cited.

### <Compound Having a Structure Represented by Formula (6)>

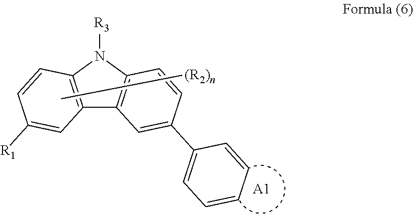

In Formula (6), R₁ represents a cyano group or CF₃.

R₂ represents an alkyl group, an aryl group, a heteroaryl group, a halogen atom, a cyano group, or a fluoroalkyl group that is substituted with any one of hydrogen atoms on carbon atoms constituting a carbazole ring. Preferably, R₂ represents an alkyl group, an aryl group, or a heteroaryl group.

R₃ represents a hydrogen atom, an alkyl group, an aryl group, a heteroaryl group, or a fluoroalkyl group. Preferably, R₃ represents an alkyl group, an aryl group, or a heteroaryl group.

n represents an integer of 0 to 6.

A1 represents a 5-membered heterocycle, provided that the 5-membered heterocycle may further have a substituent, and the substituent may form a ring. As examples of the 5-membered heterocycle and the substituent, the same ones cited for Formula (1) are cited.

### <Compound Having a Structure Represented by Formula (7)>

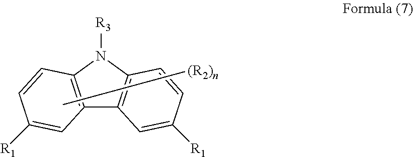

In Formula (7), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

R₃ represents a hydrogen atom, an alkyl group, an aryl group, a heteroaryl group, or a fluoroalkyl group. n represents an integer of 0 to 6.

Provided that when R₂ and R₃ each independently represent an alkyl group, an aryl group, a heteroaryl group, or a fluoroalkyl group, at least one of R₂ and R₃ contains a structure represented by Formula (2).

### <Compound Having a Structure Represented by Formula (8)>

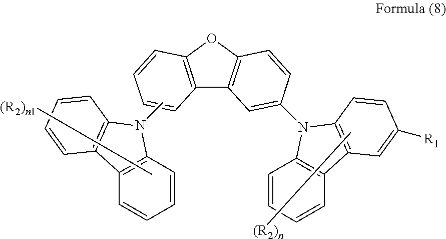

In Formula (8), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to 7.

n1 represents an integer of 0 to 8.

### <Compound Having a Structure Represented by Formula (9)>

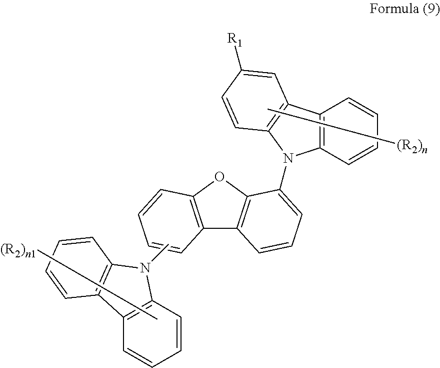

In Formula (9), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to 7.

n1 represents an integer of 0 to 8.

### <Compound Having a Structure Represented by Formula (10)>

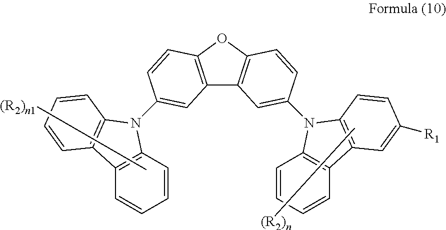

In Formula (10), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to 7.

n1 represents an integer of 0 to 8.

### <Compound Having a Structure Represented by Formula (11)>

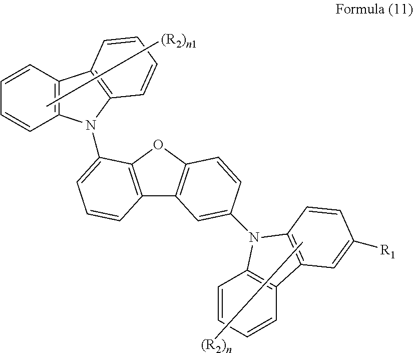

In Formula (11), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to 7.

n1 represents an integer of 0 to 8.

### <Compound Having a Structure Represented by Formula (12)>

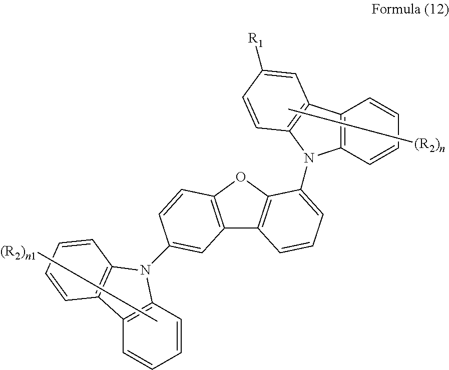

In Formula (12), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to 7.

n1 represents an integer of 0 to 8.

### <Compound Having a Structure Represented by Formula (13)>

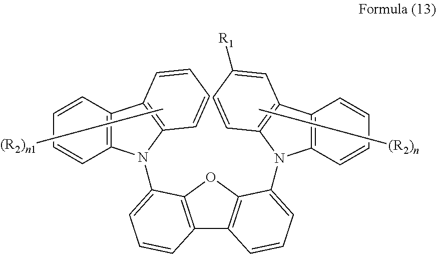

In Formula (13), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to 7.

n1 represents an integer of 0 to 8.

### <Compound Having a Structure Represented by Formula (14)>

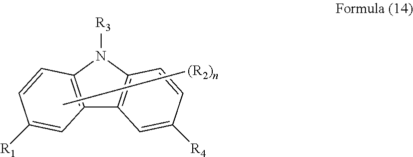

In Formula (14), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

R₃ represents a hydrogen atom, an alkyl group, an aryl group, a heteroaryl group, or a fluoroalkyl group.

R₄ represents a dibenzofuran ring.

n represents an integer of 0 to 6.

Provided that when R₂ and R₃ each independently represent an alkyl group, an aryl group, a heteroaryl group, or a fluoroalkyl group, at least one of R₂ and R₃ contains a structure represented by Formula (2).

### <Compound Having a Structure Represented by Formula (15)>

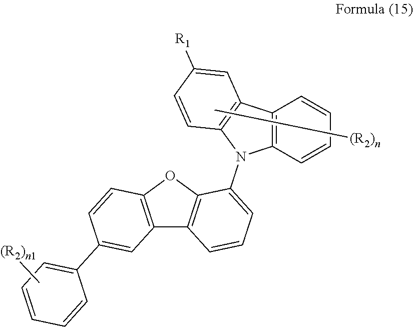

In Formula (15), R₁ represents a cyano group, CₘF₂ₘ₊₁, or SF₅.

m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to 7.

n1 represents an integer of 0 to 5.

### <Compound Having a Structure Represented by Formula (16)>

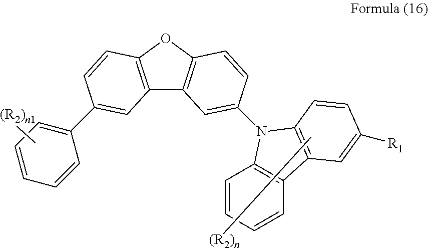

In Formula (16), R₁ represents a cyano group, CₘF₂ₘ₊₁, or SF₅. m represents an integer of 1 to 18.

R₂ represents an alkyl group, an aryl group, a heteroaryl group, a halogen atom, a cyano group, or a fluoroalkyl group that is substituted with any one of hydrogen atoms on carbon atoms constituting a carbazole ring.

n represents an integer of 0 to.

n1 represents an integer of 0 to 5.

### Specific Examples of a Compound Having a Structure Represented by Formula (1)

A specific example of a compound having a structure represented by Formula (1) is described. However, the present invention is not limited to this.

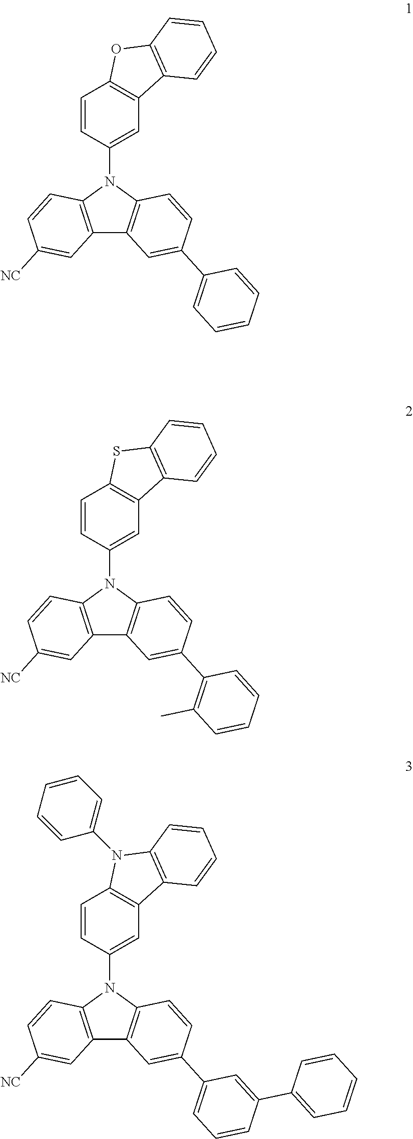 [SMILES: N#Cc1ccc2c(c1)c1cc(-c3ccccc3)ccc1n2-c1ccc2oc3ccccc3c2c1 ; Cc1ccccc1-c1ccc2c(c1)c1cc(C#N)ccc1n2-c1ccc2sc3ccccc3c2c1 ; N#Cc1ccc2c(c1)c1cc(-c3cccc(-c4ccccc4)c3)ccc1n2-c1ccc2c(c1)c1ccccc1n2-c1ccccc1]

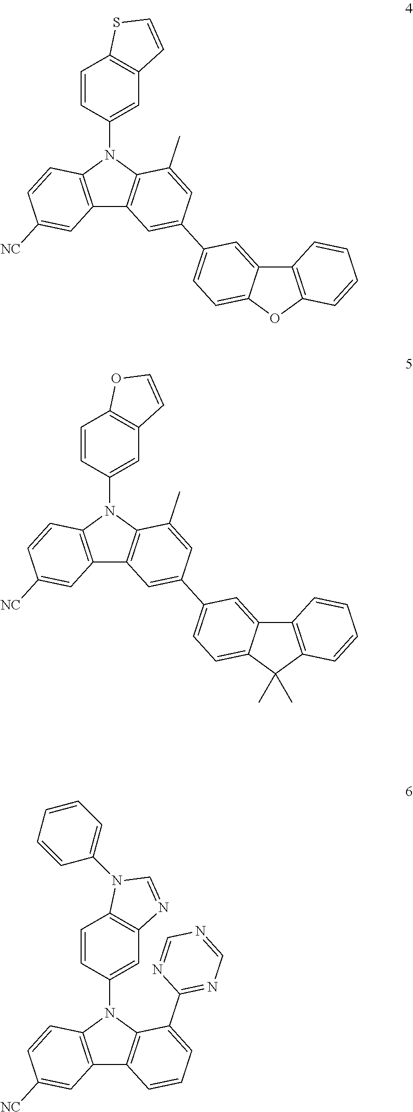 [SMILES: Cc1cc(-c2ccc3oc4ccccc4c3c2)cc2c3cc(C#N)ccc3n(-c3ccc4sccc4c3)c12 ; Cc1cc(-c2ccc3c(c2)-c2ccccc2C3(C)C)cc2c3cc(C#N)ccc3n(-c3ccc4occc4c3)c12 ; N#Cc1ccc2c(c1)c1cccc(-c3ncncn3)c1n2-c1ccc2c(c1)ncn2-c1ccccc1]

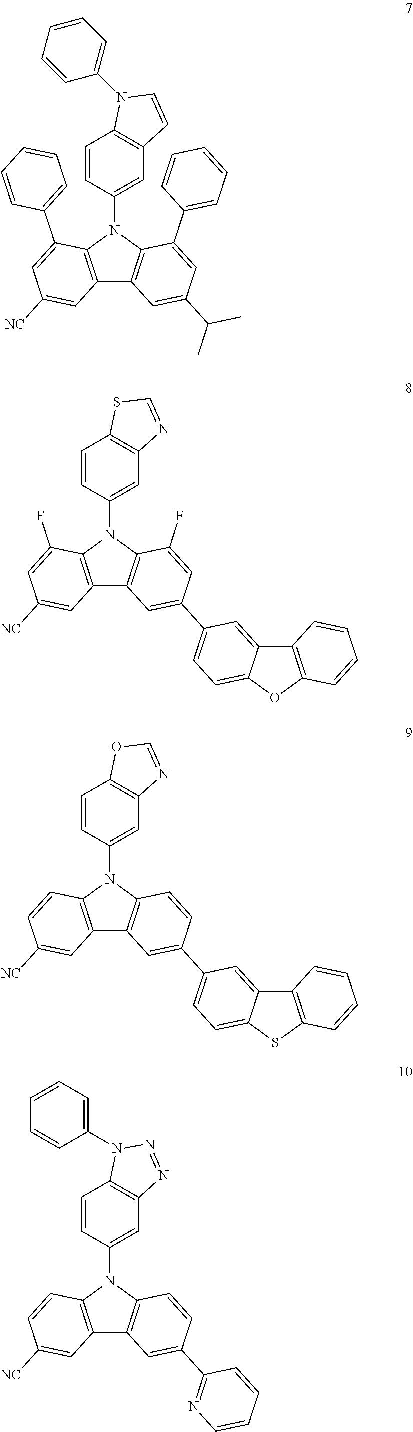 [SMILES: CC(C)c1cc(-c2ccccc2)c2c(c1)c1cc(C#N)cc(-c3ccccc3)c1n2-c1ccc2c(ccn2-c2ccccc2)c1 ; N#Cc1cc(F)c2c(c1)c1cc(-c3ccc4oc5ccccc5c4c3)cc(F)c1n2-c1ccc2scnc2c1 ; N#Cc1ccc2c(c1)c1cc(-c3ccc4sc5ccccc5c4c3)ccc1n2-c1ccc2ocnc2c1 ; N#Cc1ccc2c(c1)c1cc(-c3ccccn3)ccc1n2-c1ccc2c(c1)nnn2-c1ccccc1]

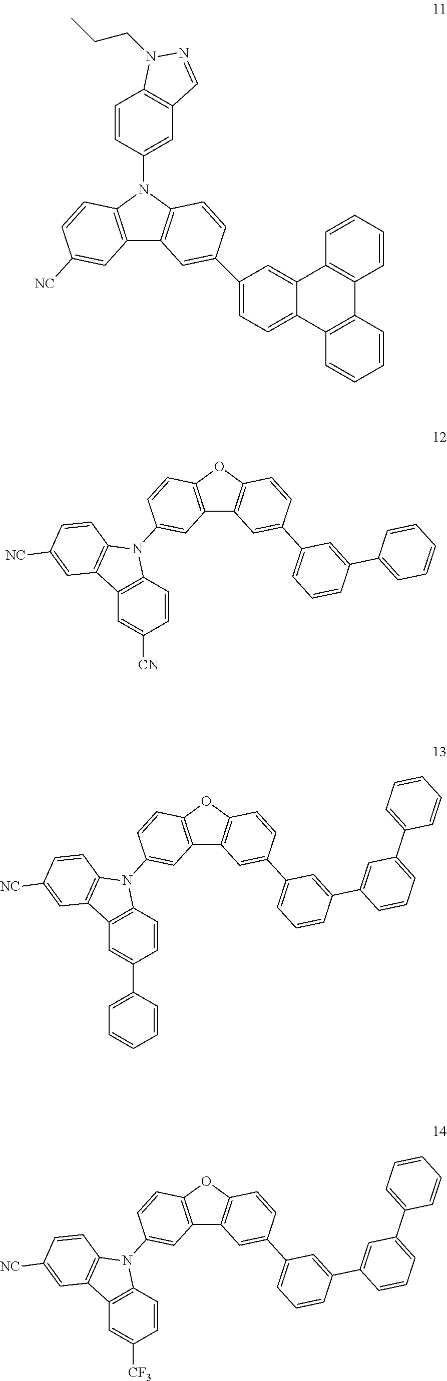 [SMILES: CCCn1ncc2cc(-n3c4ccc(C#N)cc4c4cc(-c5ccc6c7ccccc7c7ccccc7c6c5)ccc43)ccc21 ; N#Cc1ccc2c(c1)c1cc(C#N)ccc1n2-c1ccc2oc3ccc(-c4cccc(-c5ccccc5)c4)cc3c2c1 ; N#Cc1ccc2c(c1)c1cc(-c3ccccc3)ccc1n2-c1ccc2oc3ccc(-c4cccc(-c5cccc(-c6ccccc6)c5)c4)cc3c2c1 ; N#Cc1ccc2c(c1)c1cc(C(F)(F)F)ccc1n2-c1ccc2oc3ccc(-c4cccc(-c5cccc(-c6ccccc6)c5)c4)cc3c2c1]

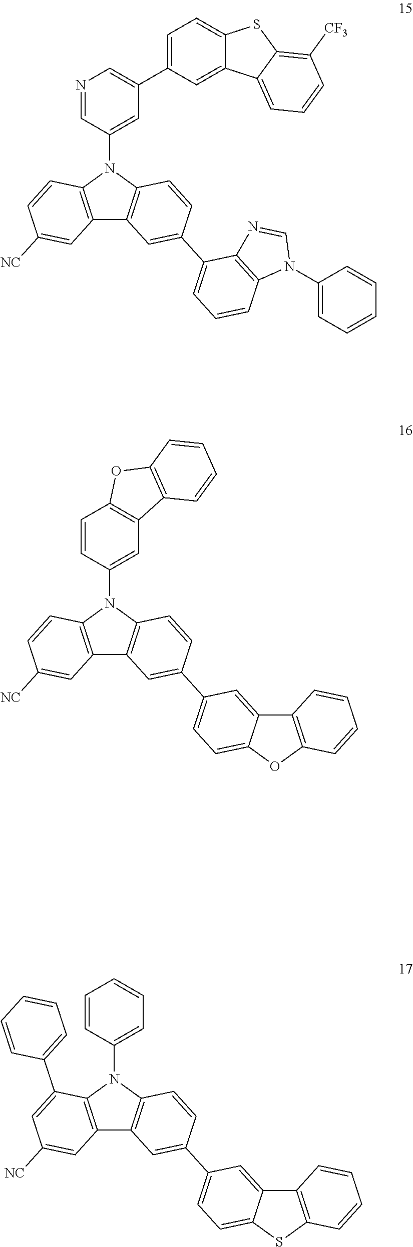 [SMILES: N#Cc1ccc2c(c1)c1cc(-c3cccc4c3ncn4-c3ccccc3)ccc1n2-c1cncc(-c2ccc3sc4c(C(F)(F)F)cccc4c3c2)c1 ; N#Cc1ccc2c(c1)c1cc(-c3ccc4oc5ccccc5c4c3)ccc1n2-c1ccc2oc3ccccc3c2c1 ; N#Cc1cc(-c2ccccc2)c2c(c1)c1cc(-c3ccc4sc5ccccc5c4c3)ccc1n2-c1ccccc1]

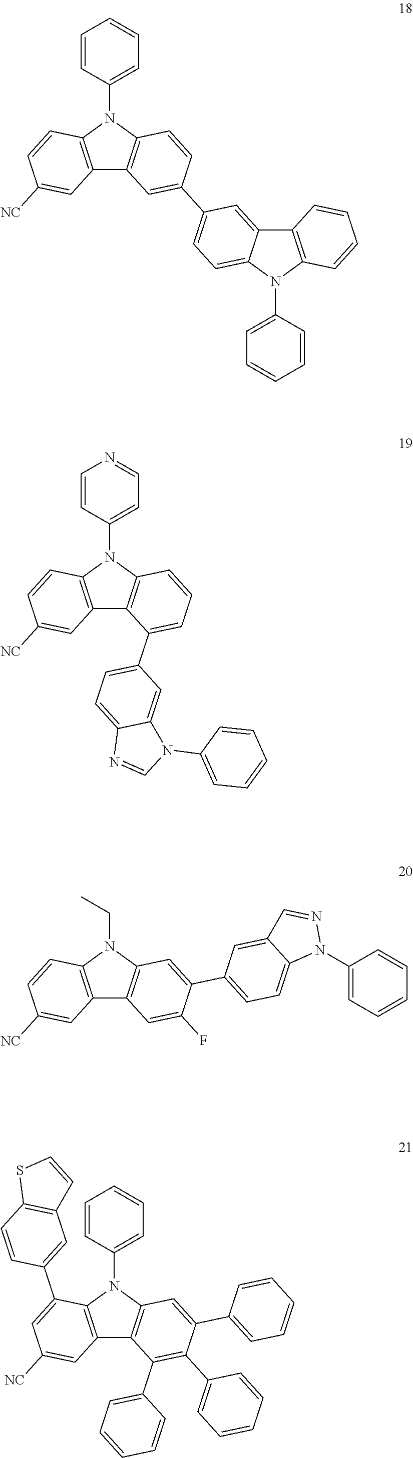 [SMILES: N#Cc1ccc2c(c1)c1cc(-c3ccc4c(c3)c3ccccc3n4-c3ccccc3)ccc1n2-c1ccccc1 ; N#Cc1ccc2c(c1)c1c(-c3ccc4ncn(-c5ccccc5)c4c3)cccc1n2-c1ccncc1 ; CCn1c2ccc(C#N)cc2c2cc(F)c(-c3ccc4c(cnn4-c4ccccc4)c3)cc21 ; N#Cc1cc(-c2ccc3sccc3c2)c2c(c1)c1c(-c3ccccc3)c(-c3ccccc3)c(-c3ccccc3)cc1n2-c1ccccc1]

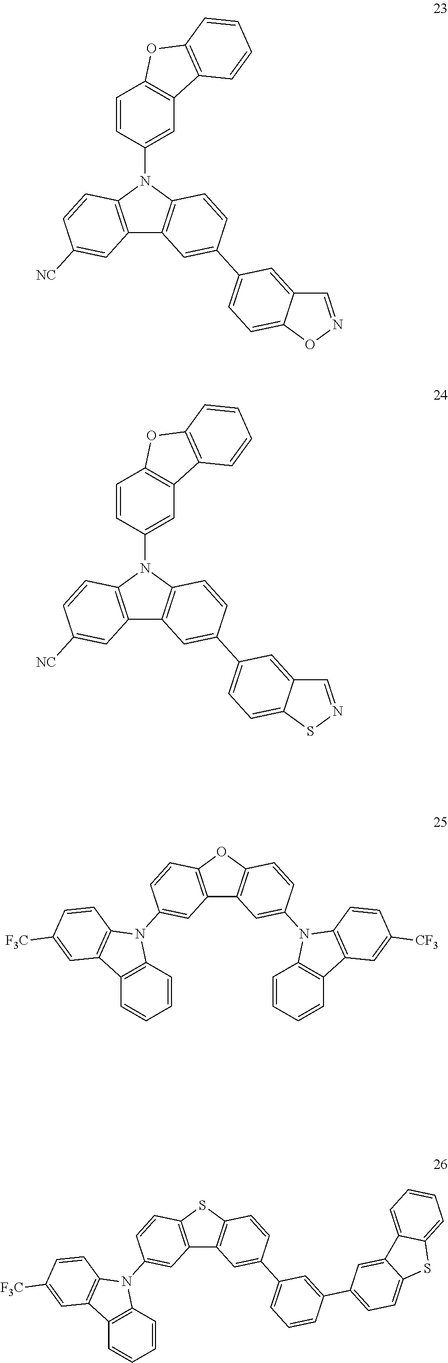 [SMILES: N#Cc1ccc2c(c1)c1cc(-c3ccc4oncc4c3)ccc1n2-c1ccc2oc3ccccc3c2c1 ; N#Cc1ccc2c(c1)c1cc(-c3ccc4sncc4c3)ccc1n2-c1ccc2oc3ccccc3c2c1 ; FC(F)(F)c1ccc2c(c1)c1ccccc1n2-c1ccc2oc3ccc(-n4c5ccccc5c5cc(C(F)(F)F)ccc54)cc3c2c1 ; FC(F)(F)c1ccc2c(c1)c1ccccc1n2-c1ccc2sc3ccc(-c4cccc(-c5ccc6sc7ccccc7c6c5)c4)cc3c2c1]

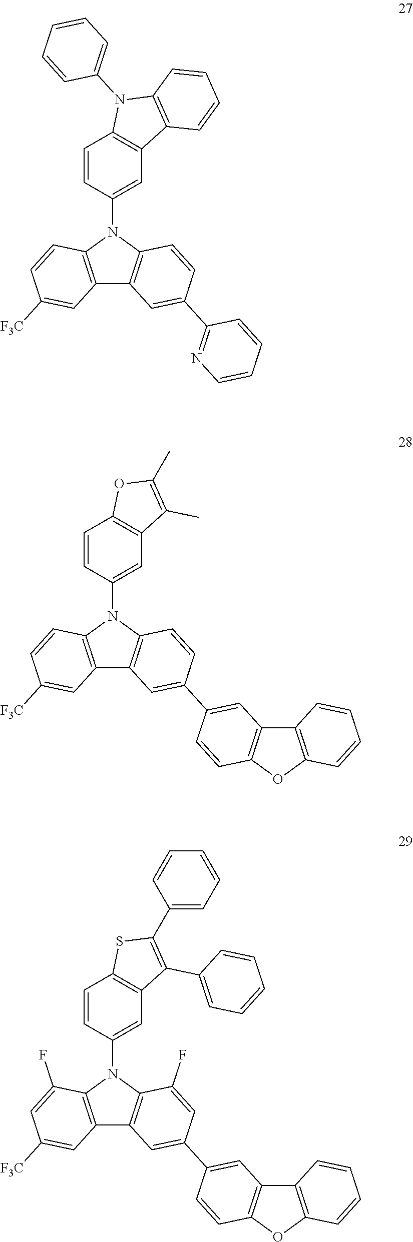 [SMILES: FC(F)(F)c1ccc2c(c1)c1cc(-c3ccccn3)ccc1n2-c1ccc2c(c1)c1ccccc1n2-c1ccccc1 ; Cc1oc2ccc(-n3c4ccc(-c5ccc6oc7ccccc7c6c5)cc4c4cc(C(F)(F)F)ccc43)cc2c1C ; Fc1cc(-c2ccc3oc4ccccc4c3c2)cc2c3cc(C(F)(F)F)cc(F)c3n(-c3ccc4sc(-c5ccccc5)c(-c5ccccc5)c4c3)c12]

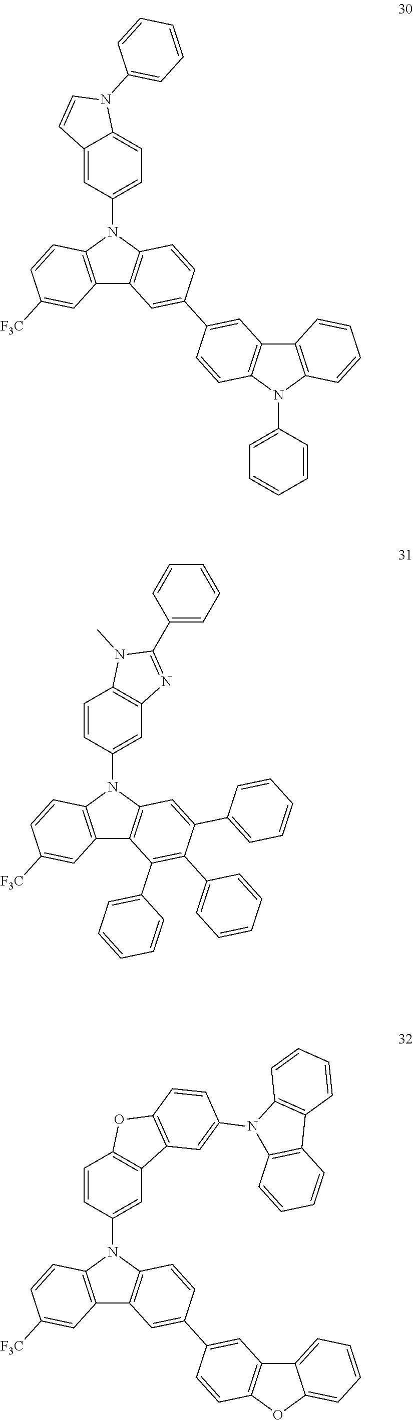 [SMILES: FC(F)(F)c1ccc2c(c1)c1cc(-c3ccc4c(c3)c3ccccc3n4-c3ccccc3)ccc1n2-c1ccc2c(ccn2-c2ccccc2)c1 ; Cn1c(-c2ccccc2)nc2cc(-n3c4ccc(C(F)(F)F)cc4c4c(-c5ccccc5)c(-c5ccccc5)c(-c5ccccc5)cc43)ccc21 ; FC(F)(F)c1ccc2c(c1)c1cc(-c3ccc4oc5ccccc5c4c3)ccc1n2-c1ccc2oc3ccc(-n4c5ccccc5c5ccccc54)cc3c2c1]

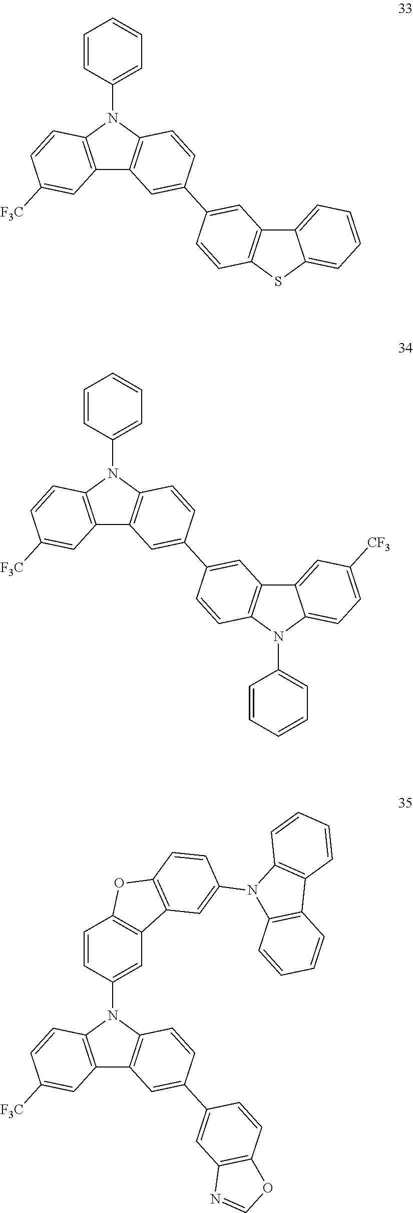 [SMILES: FC(F)(F)c1ccc2c(c1)c1cc(-c3ccc4sc5ccccc5c4c3)ccc1n2-c1ccccc1 ; FC(F)(F)c1ccc2c(c1)c1cc(-c3ccc4c(c3)c3cc(C(F)(F)F)ccc3n4-c3ccccc3)ccc1n2-c1ccccc1 ; FC(F)(F)c1ccc2c(c1)c1cc(-c3ccc4ocnc4c3)ccc1n2-c1ccc2oc3ccc(-n4c5ccccc5c5ccccc54)cc3c2c1]

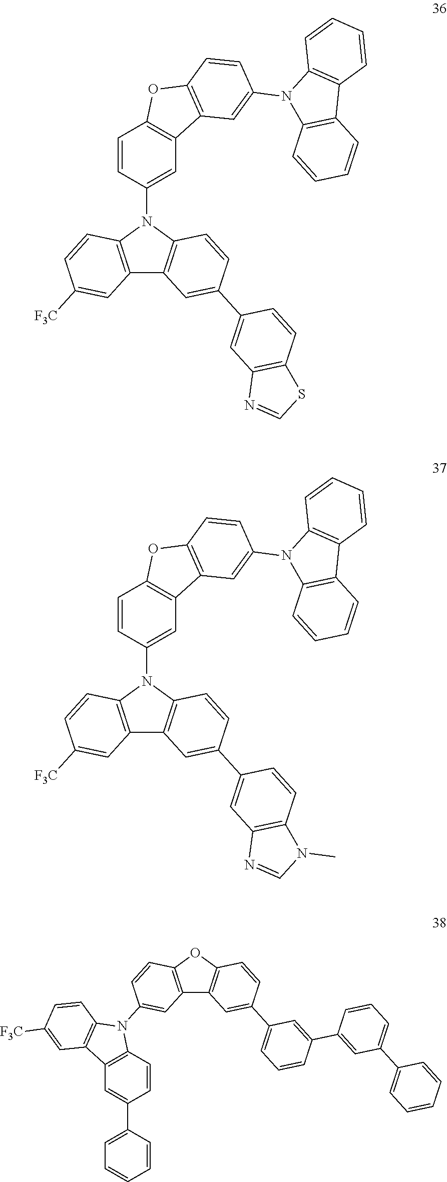 [SMILES: FC(F)(F)c1ccc2c(c1)c1cc(-c3ccc4scnc4c3)ccc1n2-c1ccc2oc3ccc(-n4c5ccccc5c5ccccc54)cc3c2c1 ; Cn1cnc2cc(-c3ccc4c(c3)c3cc(C(F)(F)F)ccc3n4-c3ccc4oc5ccc(-n6c7ccccc7c7ccccc76)cc5c4c3)ccc21 ; FC(F)(F)c1ccc2c(c1)c1cc(-c3ccccc3)ccc1n2-c1ccc2oc3ccc(-c4cccc(-c5cccc(-c6ccccc6)c5)c4)cc3c2c1]

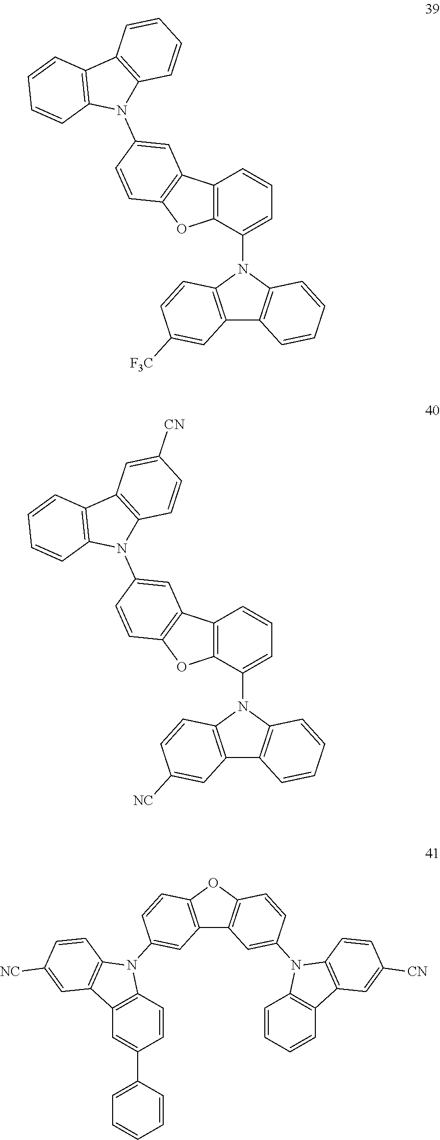 [SMILES: FC(F)(F)c1ccc2c(c1)c1ccccc1n2-c1cccc2c1oc1ccc(-n3c4ccccc4c4ccccc43)cc12 ; N#Cc1ccc2c(c1)c1ccccc1n2-c1ccc2oc3c(-n4c5ccccc5c5cc(C#N)ccc54)cccc3c2c1 ; N#Cc1ccc2c(c1)c1ccccc1n2-c1ccc2oc3ccc(-n4c5ccc(C#N)cc5c5cc(-c6ccccc6)ccc54)cc3c2c1]

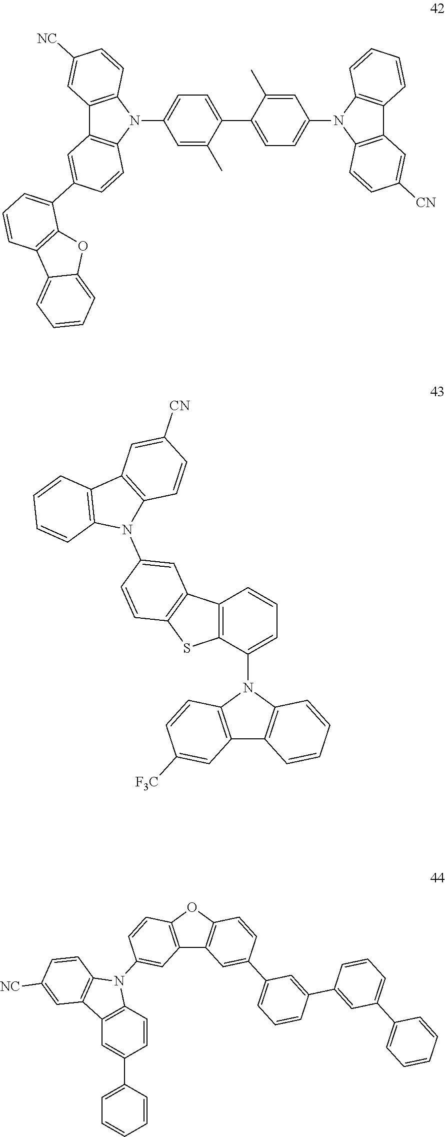 [SMILES: Cc1cc(-n2c3ccccc3c3cc(C#N)ccc32)ccc1-c1ccc(-n2c3ccc(C#N)cc3c3cc(-c4cccc5c4oc4ccccc45)ccc32)cc1C ; N#Cc1ccc2c(c1)c1ccccc1n2-c1ccc2sc3c(-n4c5ccccc5c5cc(C(F)(F)F)ccc54)cccc3c2c1 ; N#Cc1ccc2c(c1)c1cc(-c3ccccc3)ccc1n2-c1ccc2oc3ccc(-c4cccc(-c5cccc(-c6ccccc6)c5)c4)cc3c2c1]

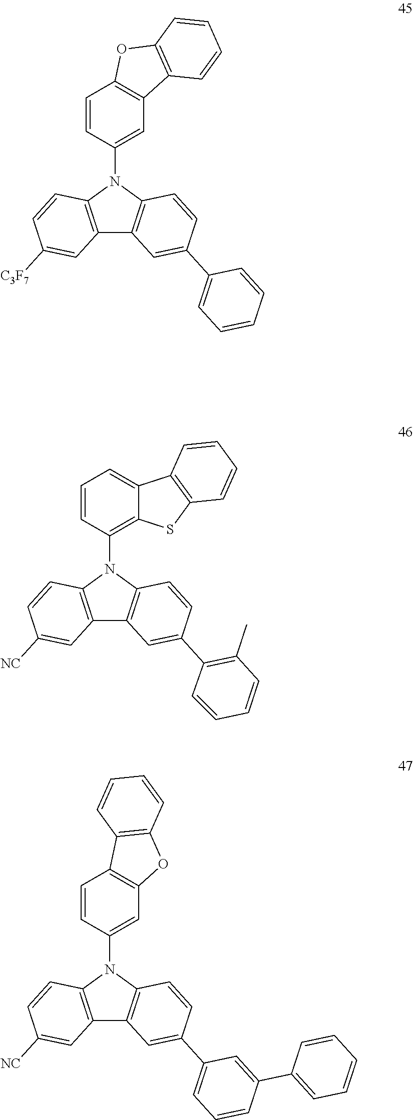

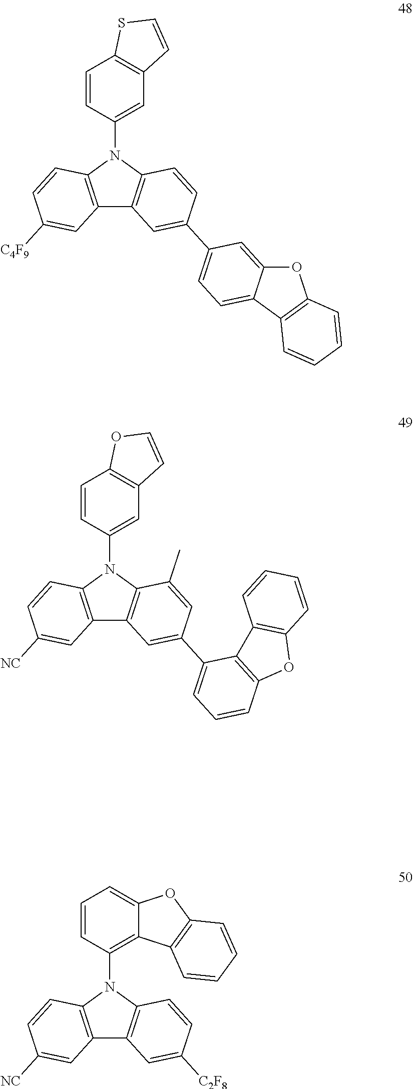

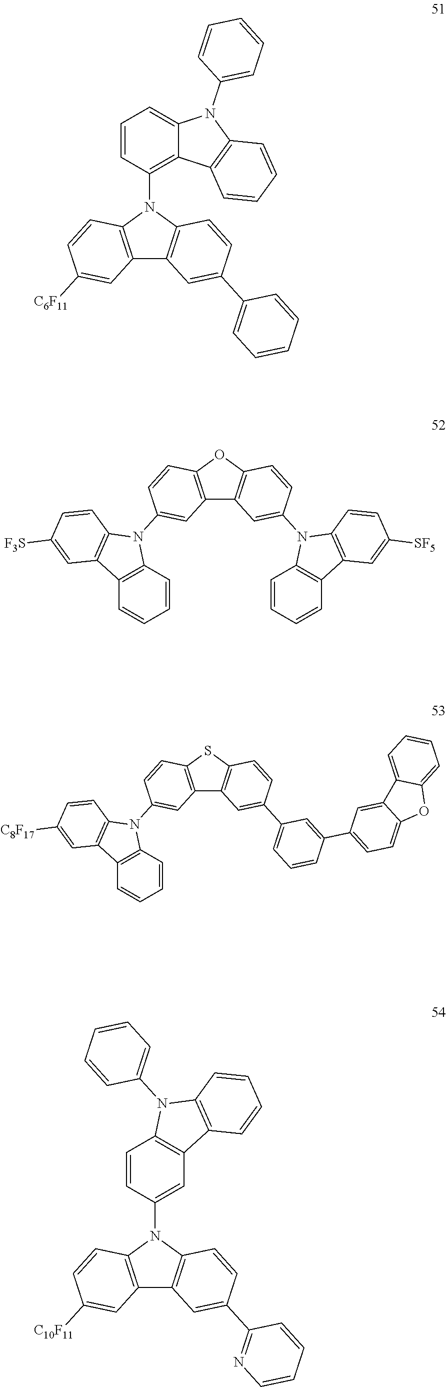

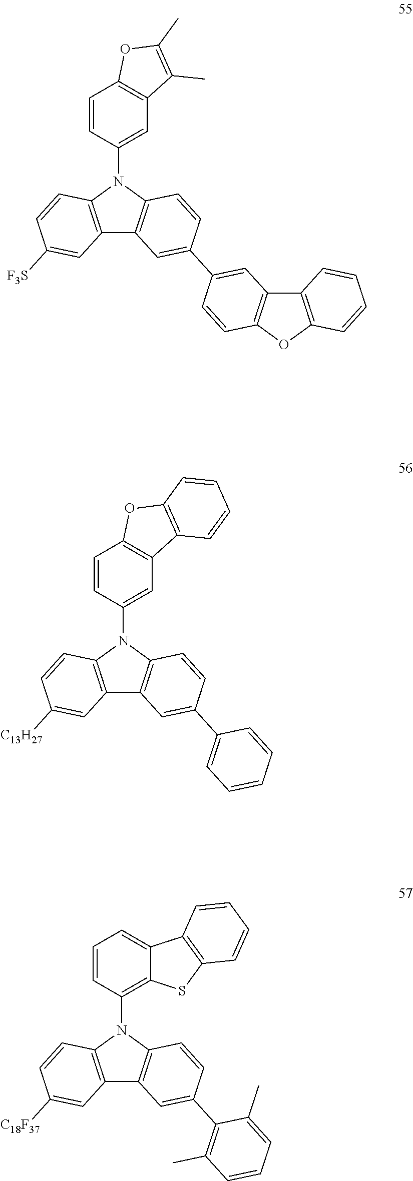

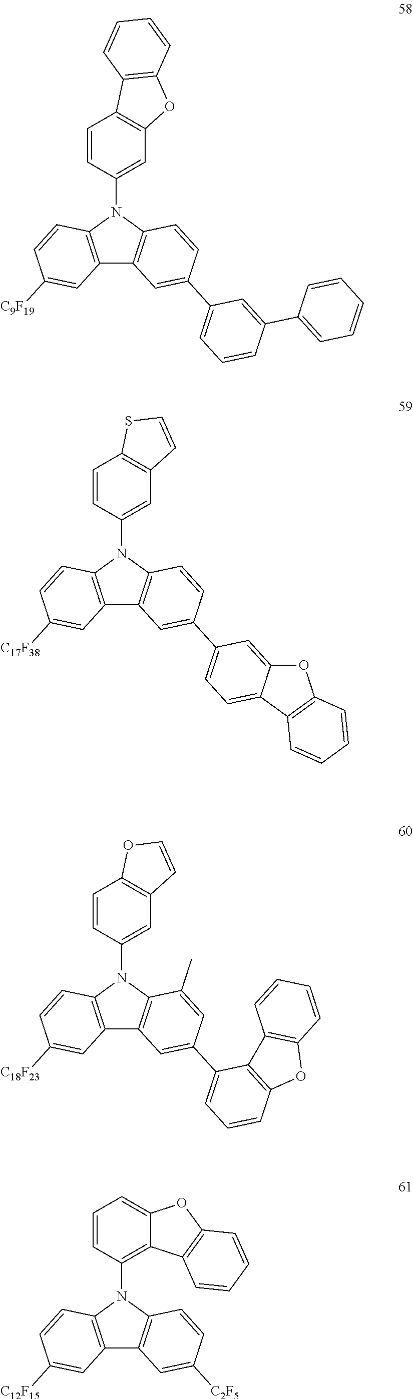

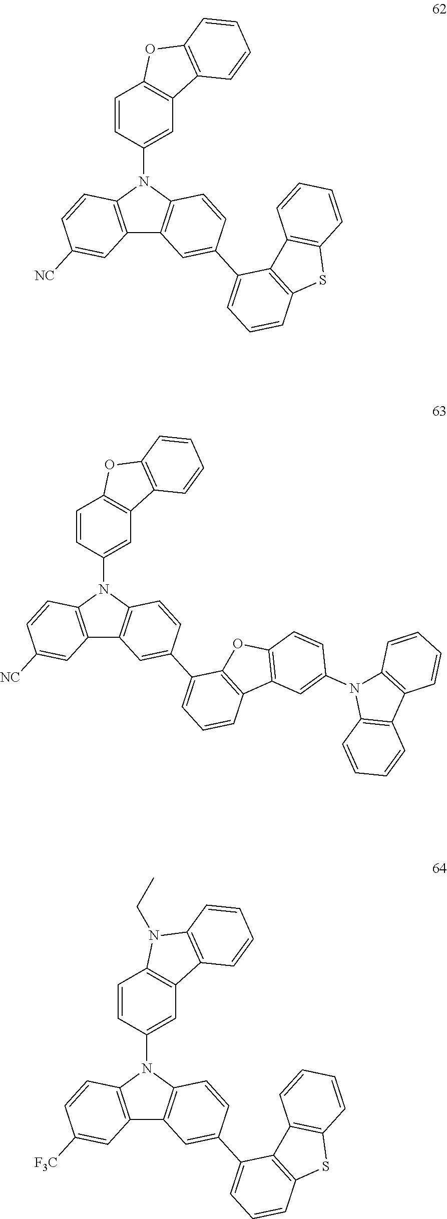 [SMILES: N#Cc1ccc2c(c1)c1cc(-c3cccc4sc5ccccc5c34)ccc1n2-c1ccc2oc3ccccc3c2c1 ; N#Cc1ccc2c(c1)c1cc(-c3cccc4c3oc3ccc(-n5c6ccccc6c6ccccc65)cc34)ccc1n2-c1ccc2oc3ccccc3c2c1 ; CCn1c2ccccc2c2cc(-n3c4ccc(-c5cccc6sc7ccccc7c56)cc4c4cc(C(F)(F)F)ccc43)ccc21]

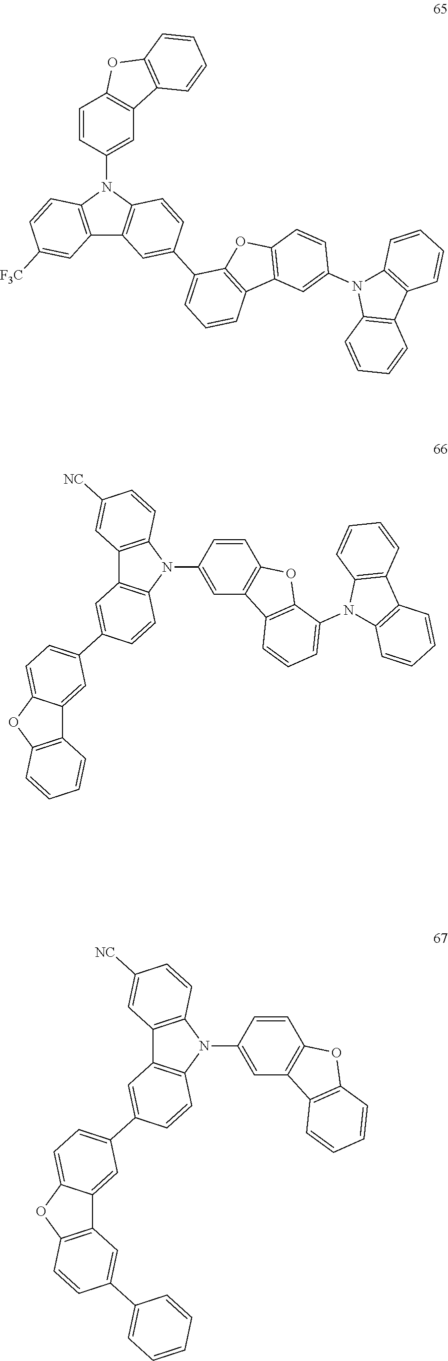 [SMILES: FC(F)(F)c1ccc2c(c1)c1cc(-c3cccc4c3oc3ccc(-n5c6ccccc6c6ccccc65)cc34)ccc1n2-c1ccc2oc3ccccc3c2c1 ; N#Cc1ccc2c(c1)c1cc(-c3ccc4oc5ccccc5c4c3)ccc1n2-c1ccc2oc3c(-n4c5ccccc5c5ccccc54)cccc3c2c1 ; N#Cc1ccc2c(c1)c1cc(-c3ccc4oc5ccc(-c6ccccc6)cc5c4c3)ccc1n2-c1ccc2oc3ccccc3c2c1]

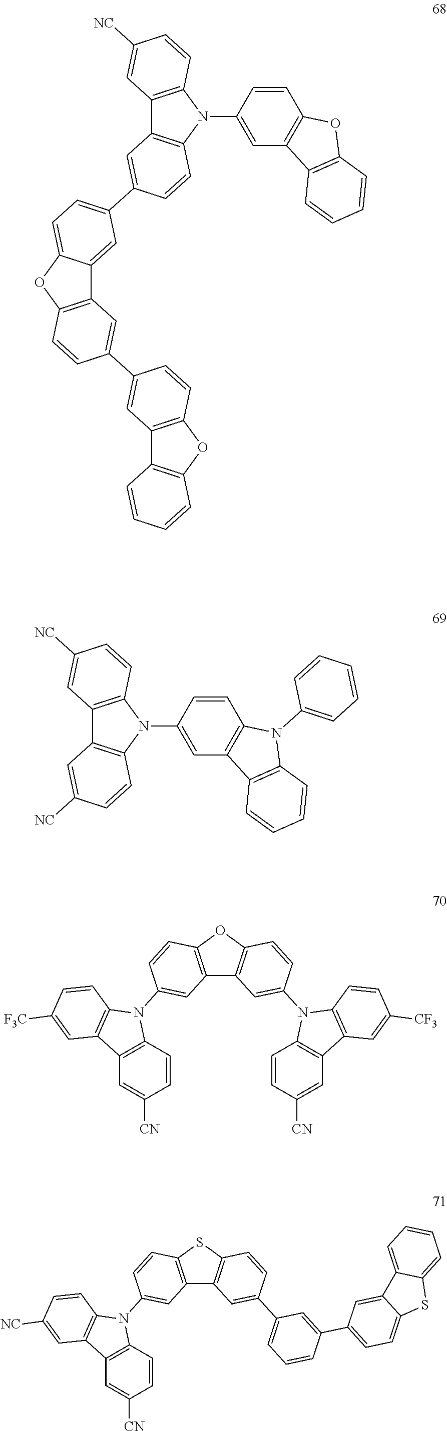 [SMILES: N#Cc1ccc2c(c1)c1cc(-c3ccc4oc5ccc(-c6ccc7oc8ccccc8c7c6)cc5c4c3)ccc1n2-c1ccc2oc3ccccc3c2c1 ; N#Cc1ccc2c(c1)c1cc(C#N)ccc1n2-c1ccc2c(c1)c1ccccc1n2-c1ccccc1 ; N#Cc1ccc2c(c1)c1cc(C(F)(F)F)ccc1n2-c1ccc2oc3ccc(-n4c5ccc(C#N)cc5c5cc(C(F)(F)F)ccc54)cc3c2c1 ; N#Cc1ccc2c(c1)c1cc(C#N)ccc1n2-c1ccc2sc3ccc(-c4cccc(-c5ccc6sc7ccccc7c6c5)c4)cc3c2c1]

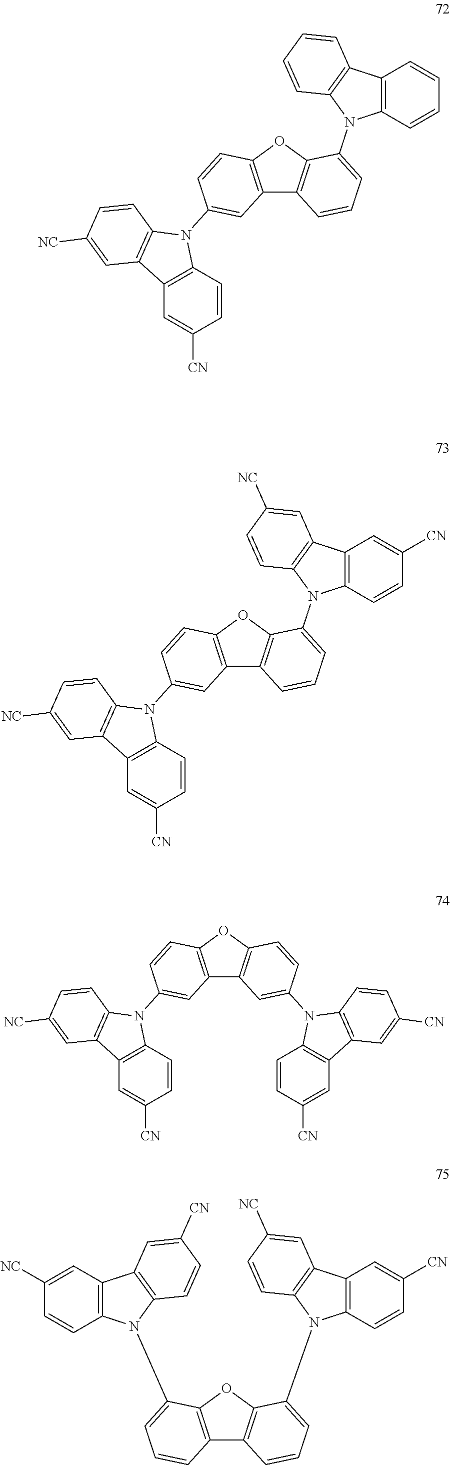 [SMILES: N#Cc1ccc2c(c1)c1cc(C#N)ccc1n2-c1ccc2oc3c(-n4c5ccccc5c5ccccc54)cccc3c2c1 ; N#Cc1ccc2c(c1)c1cc(C#N)ccc1n2-c1ccc2oc3c(-n4c5ccc(C#N)cc5c5cc(C#N)ccc54)cccc3c2c1 ; N#Cc1ccc2c(c1)c1cc(C#N)ccc1n2-c1ccc2oc3ccc(-n4c5ccc(C#N)cc5c5cc(C#N)ccc54)cc3c2c1 ; N#Cc1ccc2c(c1)c1cc(C#N)ccc1n2-c1cccc2c1oc1c(-n3c4ccc(C#N)cc4c4cc(C#N)ccc43)cccc12]

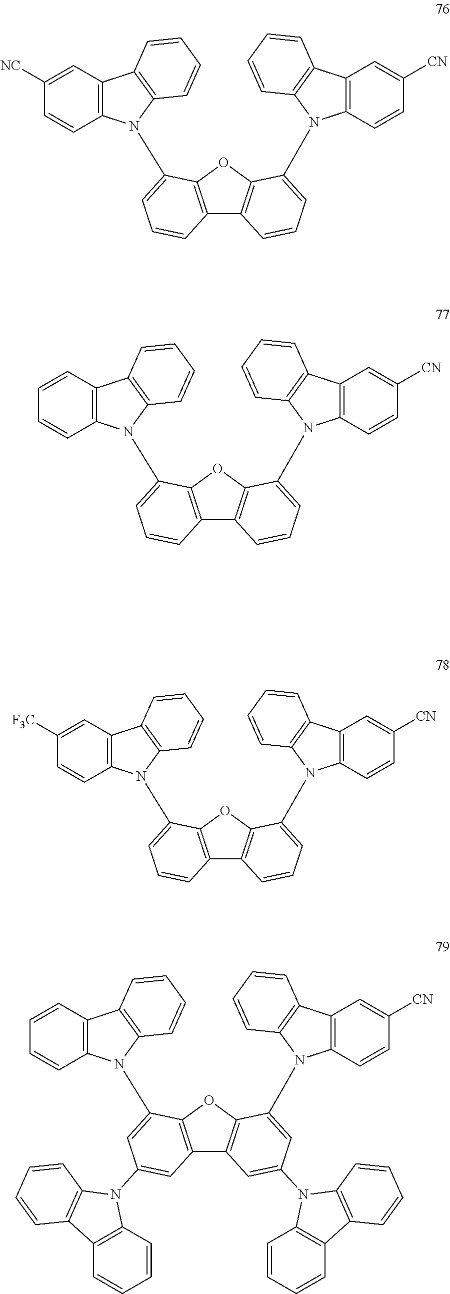 [SMILES: N#Cc1ccc2c(c1)c1ccccc1n2-c1cccc2c1oc1c(-n3c4ccccc4c4cc(C#N)ccc43)cccc12 ; N#Cc1ccc2c(c1)c1ccccc1n2-c1cccc2c1oc1c(-n3c4ccccc4c4ccccc43)cccc12 ; N#Cc1ccc2c(c1)c1ccccc1n2-c1cccc2c1oc1c(-n3c4ccccc4c4cc(C(F)(F)F)ccc43)cccc12 ; N#Cc1ccc2c(c1)c1ccccc1n2-c1cc(-n2c3ccccc3c3ccccc32)cc2c1oc1c(-n3c4ccccc4c4ccccc43)cc(-n3c4ccccc4c4ccccc43)cc12]

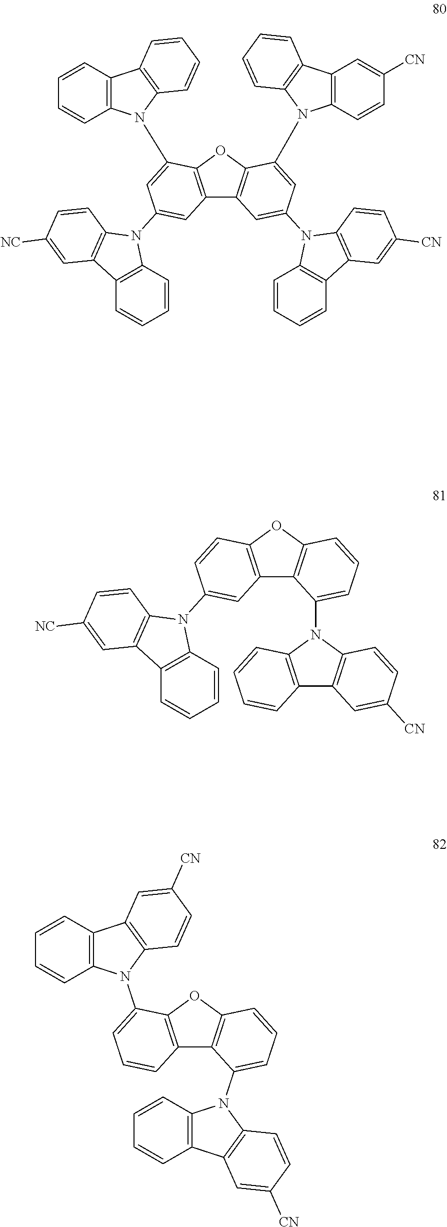 [SMILES: N#Cc1ccc2c(c1)c1ccccc1n2-c1cc(-n2c3ccccc3c3ccccc32)c2oc3c(-n4c5ccccc5c5cc(C#N)ccc54)cc(-n4c5ccccc5c5cc(C#N)ccc54)cc3c2c1 ; N#Cc1ccc2c(c1)c1ccccc1n2-c1ccc2oc3cccc(-n4c5ccccc5c5cc(C#N)ccc54)c3c2c1 ; N#Cc1ccc2c(c1)c1ccccc1n2-c1cccc2c1oc1cccc(-n3c4ccccc4c4cc(C#N)ccc43)c12]

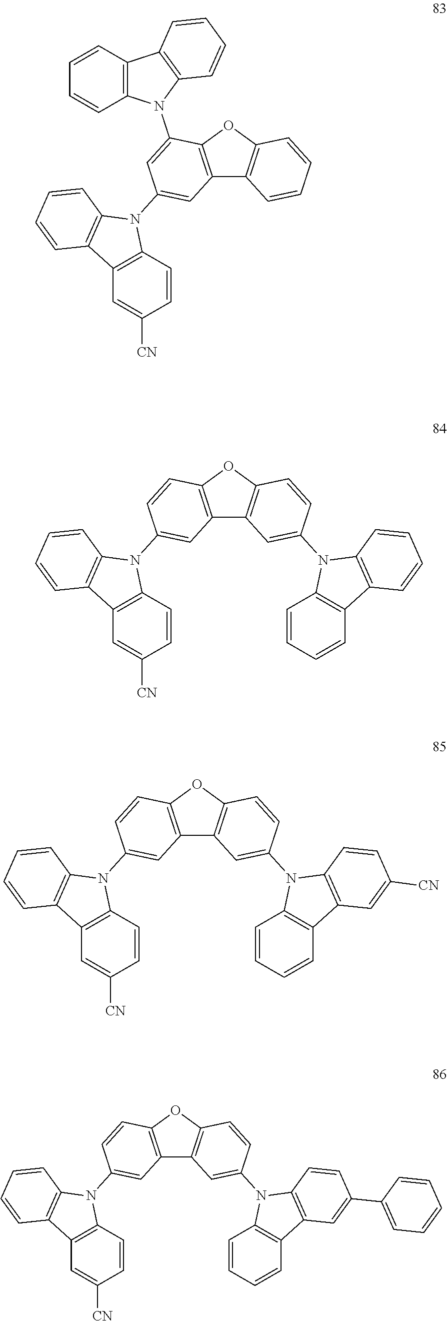

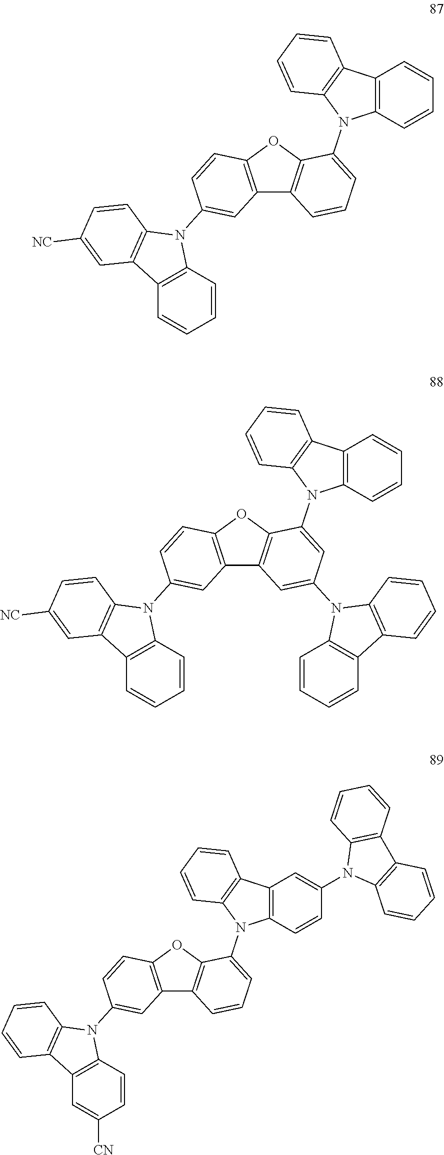

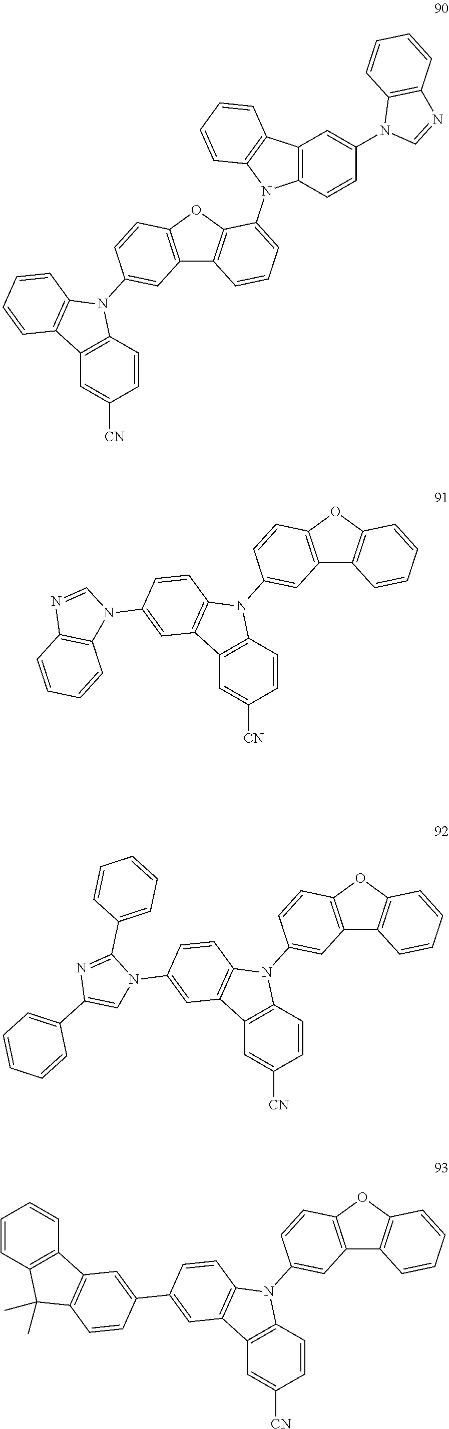

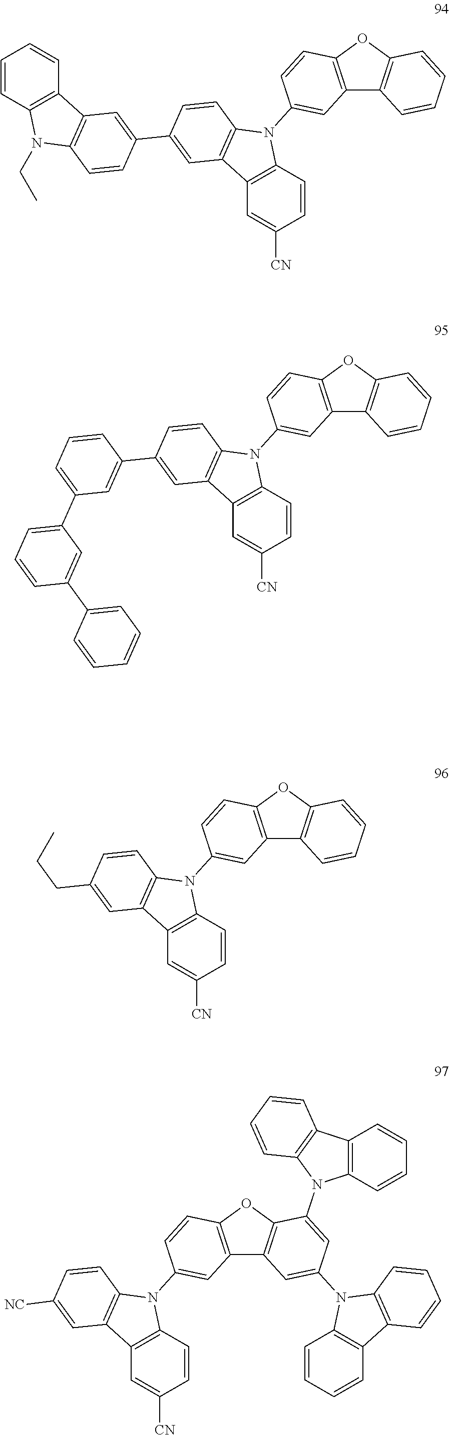 [SMILES: CCn1c2ccccc2c2cc(-c3ccc4c(c3)c3cc(C#N)ccc3n4-c3ccc4oc5ccccc5c4c3)ccc21 ; N#Cc1ccc2c(c1)c1cc(-c3cccc(-c4cccc(-c5ccccc5)c4)c3)ccc1n2-c1ccc2oc3ccccc3c2c1 ; CCCc1ccc2c(c1)c1cc(C#N)ccc1n2-c1ccc2oc3ccccc3c2c1 ; N#Cc1ccc2c(c1)c1cc(C#N)ccc1n2-c1ccc2oc3c(-n4c5ccccc5c5ccccc54)cc(-n4c5ccccc5c5ccccc54)cc3c2c1]

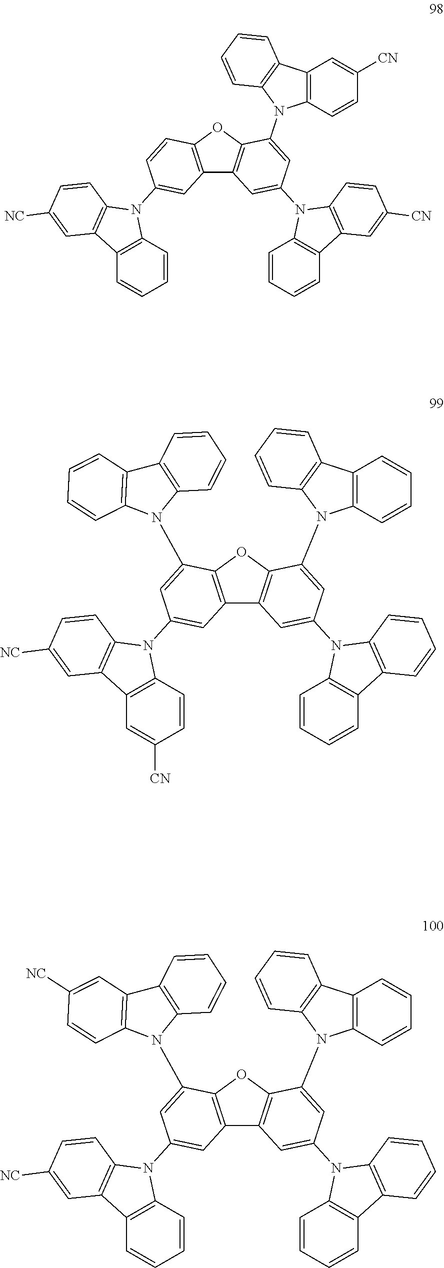 [SMILES: N#Cc1ccc2c(c1)c1ccccc1n2-c1ccc2oc3c(-n4c5ccccc5c5cc(C#N)ccc54)cc(-n4c5ccccc5c5cc(C#N)ccc54)cc3c2c1 ; N#Cc1ccc2c(c1)c1cc(C#N)ccc1n2-c1cc(-n2c3ccccc3c3ccccc32)c2oc3c(-n4c5ccccc5c5ccccc54)cc(-n4c5ccccc5c5ccccc54)cc3c2c1 ; N#Cc1ccc2c(c1)c1ccccc1n2-c1cc(-n2c3ccccc3c3cc(C#N)ccc32)c2oc3c(-n4c5ccccc5c5ccccc54)cc(-n4c5ccccc5c5ccccc54)cc3c2c1]

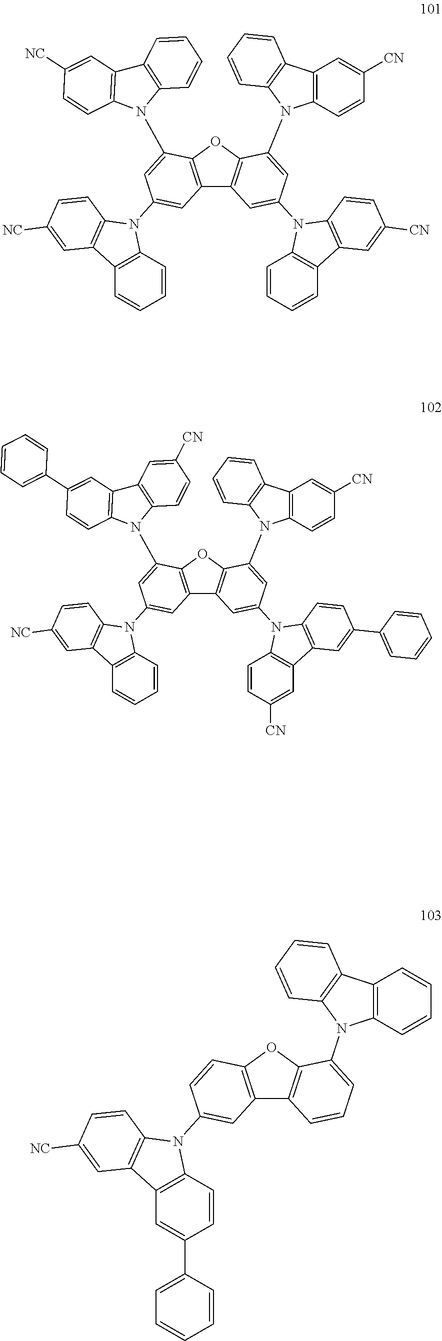 [SMILES: N#Cc1ccc2c(c1)c1ccccc1n2-c1cc(-n2c3ccccc3c3cc(C#N)ccc32)c2oc3c(-n4c5ccccc5c5cc(C#N)ccc54)cc(-n4c5ccccc5c5cc(C#N)ccc54)cc3c2c1 ; N#Cc1ccc2c(c1)c1ccccc1n2-c1cc(-n2c3ccc(C#N)cc3c3cc(-c4ccccc4)ccc32)c2oc3c(-n4c5ccccc5c5cc(C#N)ccc54)cc(-n4c5ccc(C#N)cc5c5cc(-c6ccccc6)ccc54)cc3c2c1 ; N#Cc1ccc2c(c1)c1cc(-c3ccccc3)ccc1n2-c1ccc2oc3c(-n4c5ccccc5c5ccccc54)cccc3c2c1]

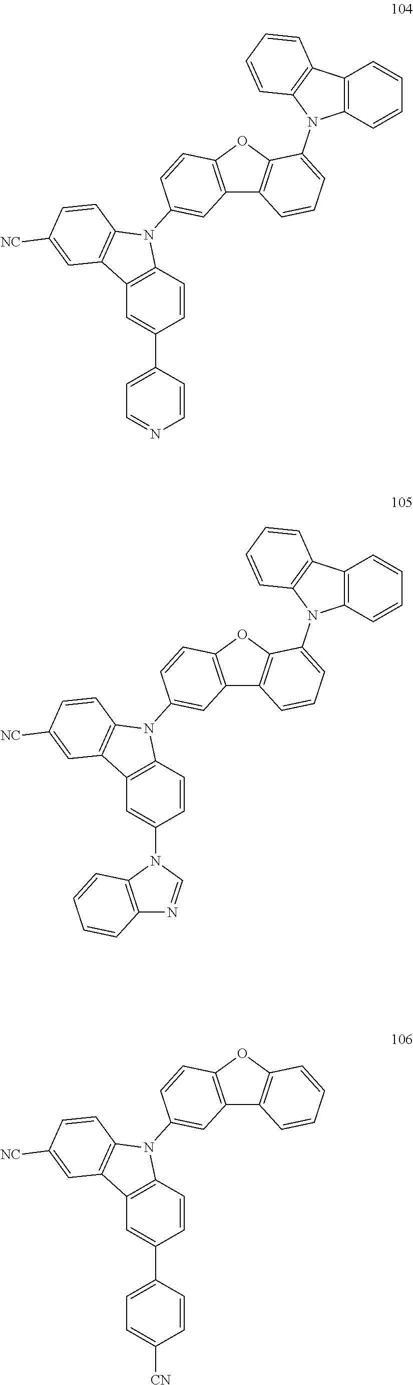 [SMILES: N#Cc1ccc2c(c1)c1cc(-c3ccncc3)ccc1n2-c1ccc2oc3c(-n4c5ccccc5c5ccccc54)cccc3c2c1 ; N#Cc1ccc2c(c1)c1cc(-n3cnc4ccccc43)ccc1n2-c1ccc2oc3c(-n4c5ccccc5c5ccccc54)cccc3c2c1 ; N#Cc1ccc(-c2ccc3c(c2)c2cc(C#N)ccc2n3-c2ccc3oc4ccccc4c3c2)cc1]

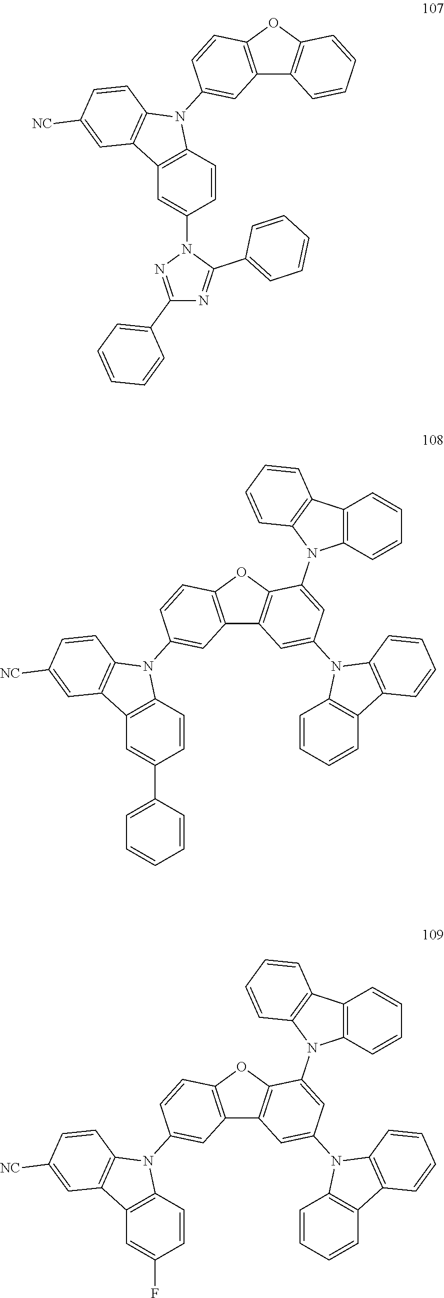 [SMILES: N#Cc1ccc2c(c1)c1cc(-n3nc(-c4ccccc4)nc3-c3ccccc3)ccc1n2-c1ccc2oc3ccccc3c2c1 ; N#Cc1ccc2c(c1)c1cc(-c3ccccc3)ccc1n2-c1ccc2oc3c(-n4c5ccccc5c5ccccc54)cc(-n4c5ccccc5c5ccccc54)cc3c2c1 ; N#Cc1ccc2c(c1)c1cc(F)ccc1n2-c1ccc2oc3c(-n4c5ccccc5c5ccccc54)cc(-n4c5ccccc5c5ccccc54)cc3c2c1]

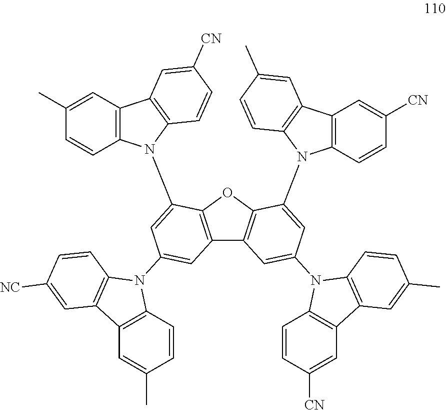 [SMILES: Cc1ccc2c(c1)c1cc(C#N)ccc1n2-c1cc(-n2c3ccc(C)cc3c3cc(C#N)ccc32)c2oc3c(-n4c5ccc(C)cc5c5cc(C#N)ccc54)cc(-n4c5ccc(C)cc5c5cc(C#N)ccc54)cc3c2c1]

### Synthetic Example of a Compound Having a Structure Represented by Formula (1)

It will be described a synthetic example of a compound having a structure represented by Formula (1) of the present invention. However, the present invention is not limited to this. Among specific examples described above, synthetic methods of exemplified compounds 38 and 38 will be described.

### Synthesis of Exemplified Compound 38

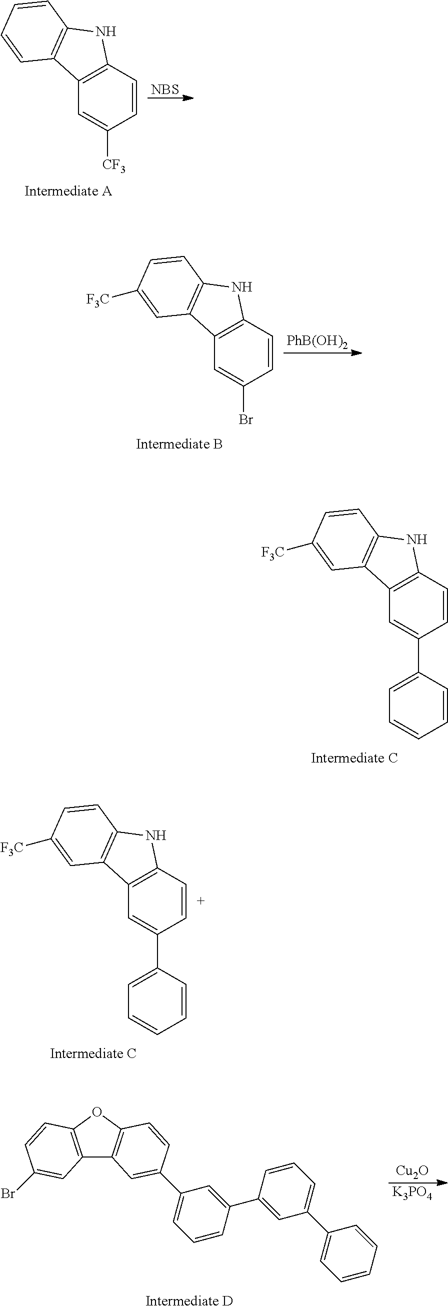

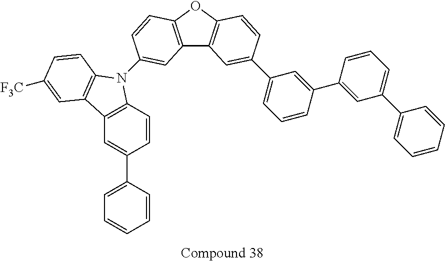 [SMILES: FC(F)(F)c1ccc2c(c1)c1cc(-c3ccccc3)ccc1n2-c1ccc2oc3ccc(-c4cccc(-c5cccc(-c6ccccc6)c5)c4)cc3c2c1]

### Step 1

In a 3-necked flask were placed 0.5 g of intermediate A and 20 mL of DMF. To this was gradually added 379 mg of NBS. Then the mixture was stirred at room temperature for 1 hour. After transferring the reaction liquid to a separation funnel, water and ethyl acetate were added, and an organic layer was extracted. The organic layer was subjected to a reduced pressured with an evaporator to remove organic solvents. The obtained residue was treated with a silica gel chromatography (developing solvent, heptane:ethyl acetate=20:1). Thus, 420 mg of intermediate B was obtained (yield 63%).

### Step 2

In a 3-necked flask were placed 420 mg of the intermediate B obtained in the step 1, 310 mg of phenyl boric acid, 15 mg of Pd(bda)₂, 78 mg of S-Phos, 10 mL of dioxane, and 1.1 g of K₃PO₄. The mixture was heated to 100° C. and stirred for 5 hours. After cooling the reaction liquid, it was transferred to a separation funnel. Then, water and ethyl acetate were added to it, and an organic layer was extracted. The organic layer was subjected to a reduced pressured with an evaporator to remove organic solvents. The obtained residue was treated with a silica gel chromatography (developing solvent, heptane:ethyl acetate=15:1). Thus, 448 mg of intermediate C was obtained (yield 63%).

### Step 3

In a 3-necked flask were placed 448 mg of the intermediate C obtained in the step 2, 652 mg of the intermediate D, 59 mg of Cu₂O, 151 mg of dipivaloyl methane, 523 mg of K₃PO₄, and 10 mL of DMS. The mixture was heated to 160° C. and stirred for 10 hours.

After transferring the reaction liquid to a separation funnel, water and ethyl acetate were added to it, and an organic layer was extracted. The organic layer was subjected to a reduced pressured with an evaporator to remove organic solvents. The obtained residue was treated with a silica gel chromatography (developing solvent, heptane:ethyl acetate=30:1). Thus, 460 mg of exemplified compound 38 was obtained (yield 45%).

The structure of compound 38 was confirmed with mass spectroscopy and ¹H-NMR. MASS Spectrum (ESI): m/z=893 (M+)

¹H-NMR (CD₂Cl₂, 400 MHz) δ: 8.50 (1H, S), δ: 8.42 (1H, s), δ: 8.22 (1H, s), δ: 8.20 (1H, d), δ: 7.92 (1H, s), δ: 7.84-7.86 (3H, m), and δ: 7.35-7.77 (22H, m).

### Synthesis of Exemplified Compound 44

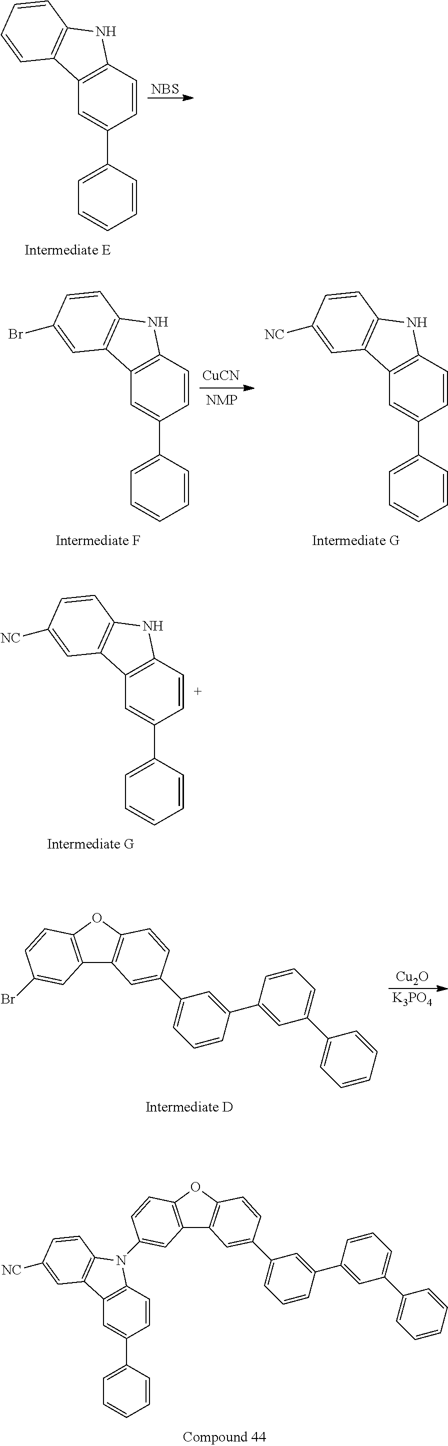

### Step 1

In a 3-necked flask were placed 3.0 g of intermediate E and 50 mL of DMF. To this was gradually added 2.3 g of NBS. Then the mixture was stirred at room temperature for 1 hour. After transferring the reaction liquid to a separation funnel, water and ethyl acetate were added, and an organic layer was extracted. The organic layer was subjected to a reduced pressured with an evaporator to remove organic solvents. The obtained residue was treated with a silica gel chromatography (developing solvent, heptane:ethyl acetate=20:1). Thus, 3.6 g of intermediate F was obtained (yield 91%).

### Step 2

In a 3-necked flask were placed 3.0 g of the intermediate F obtained in the step 1, 1.6 g of CuCN, and 25 mL of NMP. The mixture was heated to 100° C. and stirred for 5 hours. After cooling the reaction liquid, the reaction liquid was poured into a conical flask containing 50 mL of 10% aqueous hydrochloric acid and 7.3 g of FeCl₃.6H₂O. The mixture was stirred for 30 minutes at 65° C. Then, K₂CO₃ was added to the mixture to neutralize the mixture. The target compound was extracted with ethyl acetate. The organic layer was subjected to a reduced pressured with an evaporator to remove organic solvents. The obtained residue was poured into methanol and white crystal was obtained. It was filtered to obtain 1.9 g of intermediate G (yield 76%).

### Step 3

In a 3-necked flask were placed 1.0 g of the intermediate G obtained in the step 2, 1.61 g of the intermediate D, 100 mg of Cu₂O, 200 mg of dipivaloyl methane, 1.3 g of K₃PO₄, and 25 mL of DMS. The mixture was heated to 140° C. and stirred for 7 hours.

After transferring the reaction liquid to a separation funnel, water and ethyl acetate were added to it, and an organic layer was extracted. The organic layer was subjected to a reduced pressured with an evaporator to remove organic solvents. The obtained residue was treated with a silica gel chromatography (developing solvent, heptane:ethyl acetate=20:1). Thus, 0.69 g of exemplified compound 44 was obtained (yield 41%).

The structure of compound 44 was confirmed with mass spectroscopy and ¹H-NMR.

MASS Spectrum (ESI): m/z=893 (M+)

¹H-NMR (CD₂Cl₂, 400 MHz) δ: 8.51 (1H, S), δ: 8.38 (1H, d), δ: 8.22 (1H, s), δ: 8.18 (1H, d), δ: 7.85 (1H, s), δ: 7.91-7.85 (3H, m), and δ: 7.38-7.77 (22H, m).

### [Constituting Layers of Organic EL Element]

An organic EL element of the present invention is characterized in containing the above-described organic EL element materials.

Constituting layers of an organic EL element of the present invention will be described. In an organic EL element of the present invention, preferable examples of a layer constitution of various organic layers interposed between a cathode and an anode are indicated below. However, the present invention is not limited to them.

(i) Anode/light emitting layer unit/electron transport layer/cathode
(ii) Anode/hole transport layer/light emitting layer unit/electron transport layer/cathode
(iii) Anode/hole transport layer/light emitting layer unit/hole blocking layer/electron transport layer/cathode
(iv) Anode/hole transport layer/light emitting layer unit/hole blocking layer/electron transport layer/cathode buffer layer/cathode
(v) Anode/anode buffer layer/hole transport layer/light emitting layer unit/hole blocking layer/electron transport layer/cathode buffer layer/cathode

The light emitting layer unit may have a non-light emitting intermediate layer between a plurality of light emitting layers. It may have a multi-photon unit structure having a charge generation layer as an interlayer.

Examples of a charge generation layer composed of a conductive organic compounds such as:

Examples of a material used for a charge generation layer are: conductive inorganic compounds such as ITO (indium tin oxide), IZO (indium zinc oxide), ZnO₂, TiN, ZrN, HfN, TiOx, VOx, CuI, InN, GaN, CuAlO₂, CuGaO₂, SrCu₂O₂, LaB₆, RuO₂, and Al; a two-layer film such as Au/Bi₂O₃; a multi-layer film such as SnO₂/Ag/SnO₂, ZnO/Ag/ZnO, Bi₂O₃/Au/Bi₂O₃, TiO₂/TiN/TiO₂, and TiO₂/ZrN/TiO₂; fullerene such as C₆₀; and a conductive organic layer such as oligothiophene, metal phthalocyanine, metal-free phthalocyanine, metal porphyrin, and metal-free porphyrin.

As a light emitting layer of an organic EL element of the present invention, it is preferable that it is a blue light emitting layer or a white light emitting layer. It is preferable that a lighting device uses these elements.

Each layer that constitutes an organic EL element of the present invention will be described in the following.

### <Light Emitting Layer>

A light emitting layer relating to the present invention is a layer which provide a place of emitting light via an exciton produce by recombination of electrons and holes injected from an electrode or an adjacent layer. The light emitting portion may be either within the light emitting layer or at an interface between the light emitting layer and an adjacent layer thereof.

A total thickness of the light emitting layer is not particularly limited. However, in view of layer homogeneity, required voltage during light emission, and stability of the emitted light color against a drive electric current, a layer thickness is preferably adjusted to be in the range of 2 nm to 5 μm, more preferably, it is in the range of 2 to 200 nm, and still most preferably, it is in the range of 5 to 100 nm.

The light emitting layer may be formed with an emission dopant and a host compound, which are described later, by using a method such as a vacuum vapor deposition method and a wet method.

Examples of a wet process include: a spin coating method, a cast method, a die coating method, a blade coating method, a roll coating method, an inkjet method, a printing method, a spray coating method, a curtain coating method, and a LB method (Langmuir Blodgett method). It is preferable that a light emitting layer of an organic EL element of the present invention contain an emission dopant compound (a phosphorescence emitting dopant or a fluorescence emitting dopant) and a host compound.

### (1. Emission Dopant)

It will be described an emission dopant (it may be called as: emitting dopant, a dopant compound, or simply a dopant).

As an emission dopant, it may be used: a fluorescence emitting dopant (also referred to as a fluorescent dopant and a fluorescent compound) and a phosphorescence emitting dopant (also referred to as a phosphorescent dopant and a phosphorescent emitting material).

A concentration of an emission dopant in a light emitting layer may be arbitrarily decided based on the specific compound employed and the required conditions of the device. A concentration of an emission dopant may be uniform in a thickness direction of the light emitting layer, or it may have any concentration distribution.

The light emitting layer may contain a plurality of emission dopants. For example, it may be used a combination of dopants each having a different structure, or a combination of a fluorescence emitting dopant and a phosphorescence emitting dopant. By this, an arbitral emission color will be obtained.

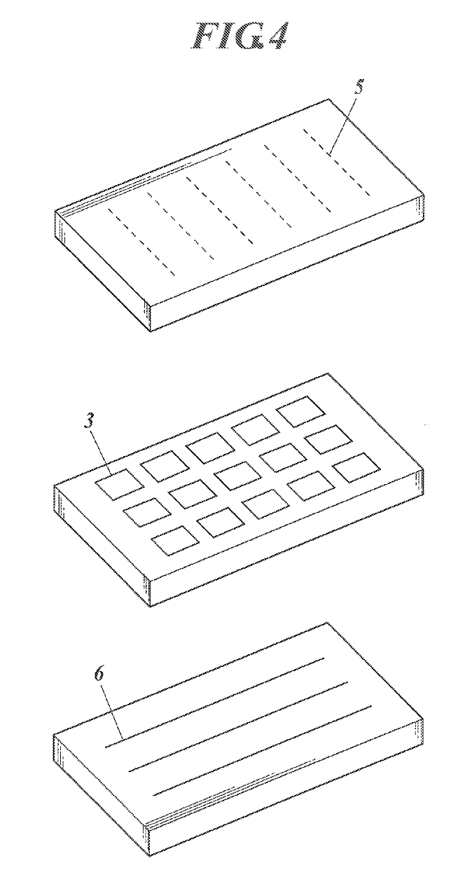

Color of light emitted by an organic EL element is specified as follows. In FIG. 4.16 on page 108 of “Shinpen Shikisai Kagaku Handbook (New Edition Color Science Handbook)” (edited by The Color Science Association of Japan, Tokyo Daigaku Shuppan Kai, 1985), values determined via a spectroradiometer CS-2000 (produced by Konica Minolta, Inc.) are applied to the CIE 1931 chromaticity coordinate, whereby the color is specified.

It is preferable that an organic EL element has one or more light emitting layers that contain a plurality of emission dopants each emits a light of a different color, and to emit white light. There is no specific limitation to the combination of emission dopants to emit white light. It may be cited a combination of: blue and orange; and blue, green and red.

It is preferable that “white” in the organic EL element of the present invention shows chromaticity in the CIE 1931 Color Specification System at 1,000 cd/m² in the region of x=0.39±0.09 and y=0.38±0.08, when measurement is done to 2-degree viewing angle front luminance via the aforesaid method.

### (1-1. Phosphorescence Emitting Dopant)

The phosphorescence emitting dopant is a compound which is observed emission from an excited triplet state thereof. Specifically, it is a compound which emits phosphorescence at a room temperature (25° C.) and exhibits a phosphorescence quantum yield of at least 0.01 at 25° C. The phosphorescence quantum yield is preferably at least 0.1.

The phosphorescence quantum yield will be determined via a method described in page 398 of Bunko II of Dai 4 Han Jikken Kagaku Koza 7 (Spectroscopy II of 4th Edition Lecture of Experimental Chemistry 7) (1992, published by Maruzen Co. Ltd.). The phosphorescence quantum yield in a solution will be determined using appropriate solvents. However, it is only necessary for the phosphorescent dopant of the present invention to exhibit the above phosphorescence quantum yield (0.01 or more) using any of the appropriate solvents.

Two kinds of principles regarding emission of a phosphorescent dopant are cited. One is an energy transfer-type, wherein carriers recombine on a host compound on which the carriers are transferred to produce an excited state of the host compound, and then via transfer of this energy to a phosphorescent dopant, emission from the phosphorescence-emitting dopant is realized. The other is a carrier trap-type, wherein a phosphorescence-emitting dopant serves as a carrier trap and then carriers recombine on the phosphorescent dopant to generate emission from the phosphorescent dopant. In each case, the excited state energy level of the phosphorescent dopant is required to be lower than that of the host compound.

A phosphorescence dopant may be suitably selected and employed from the known materials used for a light emitting layer for an organic EL element.

Examples of a known phosphorescence dopant are compound described in the following publications.

Nature 395, 151 (1998), Appl. Phys. Lett. 78, 1622 (2001), Adv. Mater. 19, 739 (2007), Chem. Mater. 17, 3532 (2005), Adv. Mater. 17, 1059 (2005), WO 2009/100991, WO 2008/101842, WO 2003/040257, US 2006/0202194, US 2007/0087321, and US 2005/0244673.

Inorg. Chem. 40, 1704 (2001), Chem. Mater. 16, 2480 (2004), Adv. Mater. 16, 2003 (2004), Angew. Chem. Int. Ed. 2006, 45, 7800, Appl. Phys. Lett. 86, 153505 (2005), Chem. Lett. 34, 592 (2005), Chem. Commun. 2906 (2005), Inorg. Chem. 42, 1248 (2003), WO 2009/050290, WO 2002/015645, WO 2009/000673, US 2002/0034656, U.S. Pat. No. 7,332,232, US 2009/0108737, US 2009/0039776, U.S. Pat. No. 6,921,915, U.S. Pat. No. 6,687,266, US 2007/0190359, US 2006/0008670, US 2009/0165846, US 2008/0015355, U.S. Pat. No. 7,250,226, U.S. Pat. No. 7,396,598, US 2006/0263635, US 2003/0138657, US 2003/0152802, and U.S. Pat. No. 7,090,928.

Angew. Chem. Int. Ed. 47, 1 (2008), Chem. Mater. 18, 5119 (2006), Inorg. Chem. 46, 4308 (2007), Organometallics 23, 3745 (2004), Appl. Phys. Lett. 74, 1361 (1999), WO 2002/002714, WO 2006/009024, WO 2006/056418, WO 2005/019373, WO 2005/123873, WO 2005/123873, WO 2007/004380, WO 2006/082742, US 2006/0251923, US 2005/0260441, U.S. Pat. No. 7,393,599, U.S. Pat. No. 7,534,505, U.S. Pat. No. 7,445,855, US 2007/0190359, US 2008/0297033, U.S. Pat. No. 7,338,722, US 2002/0134984, and U.S. Pat. No. 7,279,704.

WO 2005/076380, WO 2010/032663, WO 2008/140115, WO 2007/052431, WO 2011/134013, WO 2011/157339, WO 2010/086089, WO 2009/113646, WO 2012/020327, WO 2011/051404, WO 2011/004639, WO 2011/073149, US 2012/228583, US 2012/212126, JP-A No. 2012-069737, JP-A No. 2012-195554, JP-A No. 2009-114086, JP-A No. 2003-81988, JP-A No. 2002-302671 and JP-A No. 2002-363552.

Among them, preferable phosphorescence emitting dopants are organic metal complexes containing Ir as a center metal. More preferable are complexes containing at least one coordination mode selected from a metal-carbon bond, a metal-nitrogen bond, a metal-oxygen bond and a metal-sulfur bond.

Specific examples of a known phosphorescence emitting dopant that is applicable to a light emitting layer are described in the following. However, the phosphorescence emitting dopants are not limited to them, other compounds may be applied.

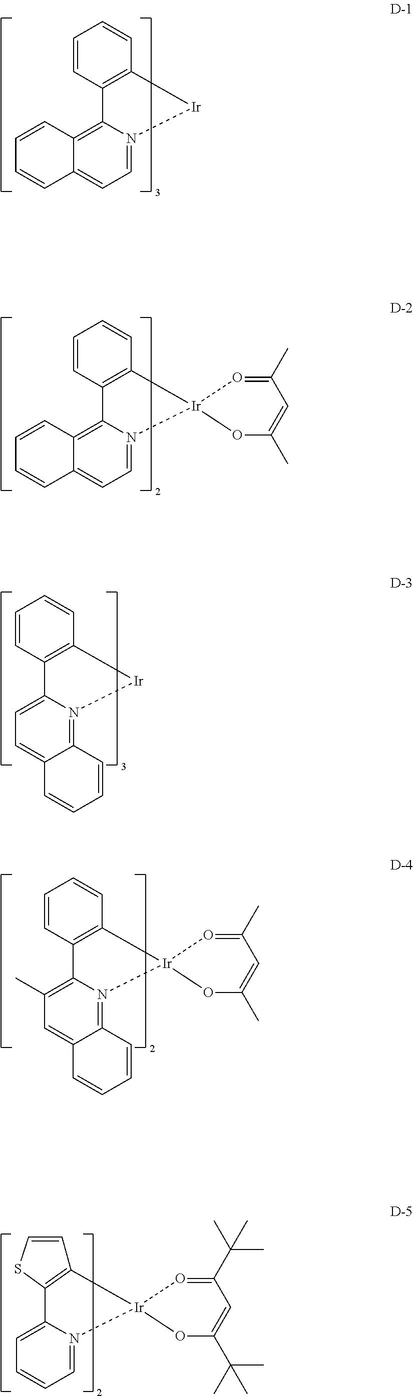

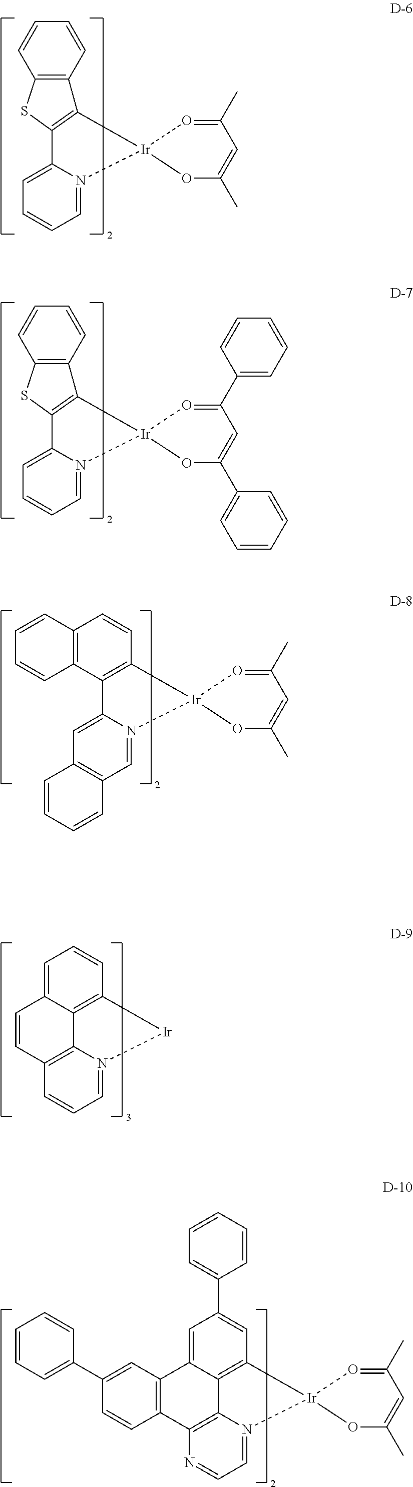

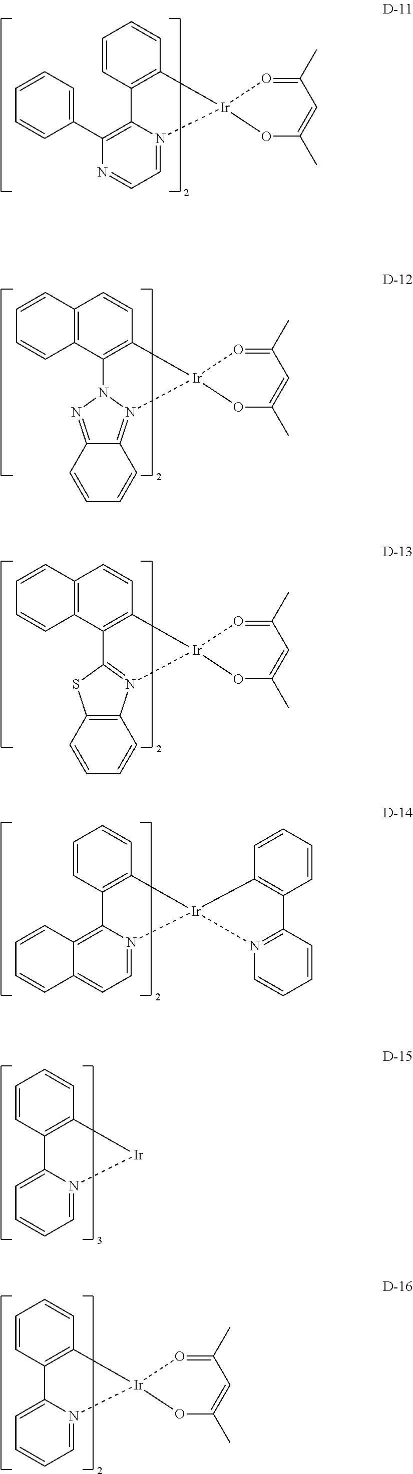

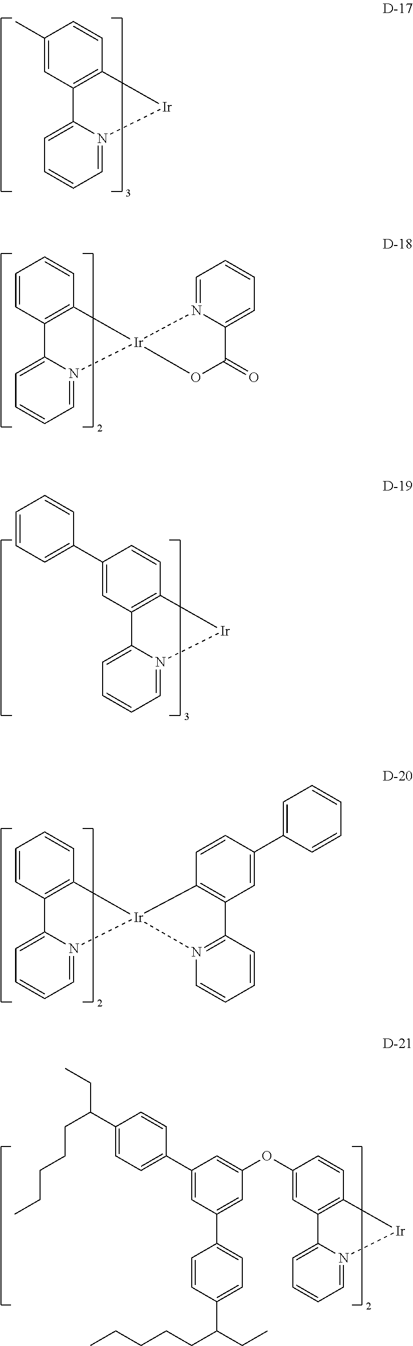

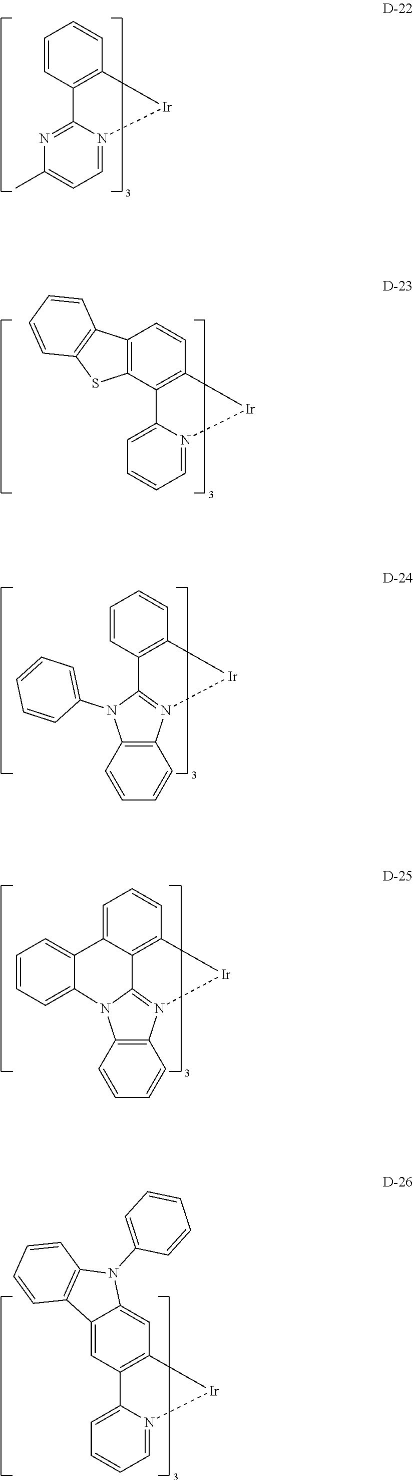

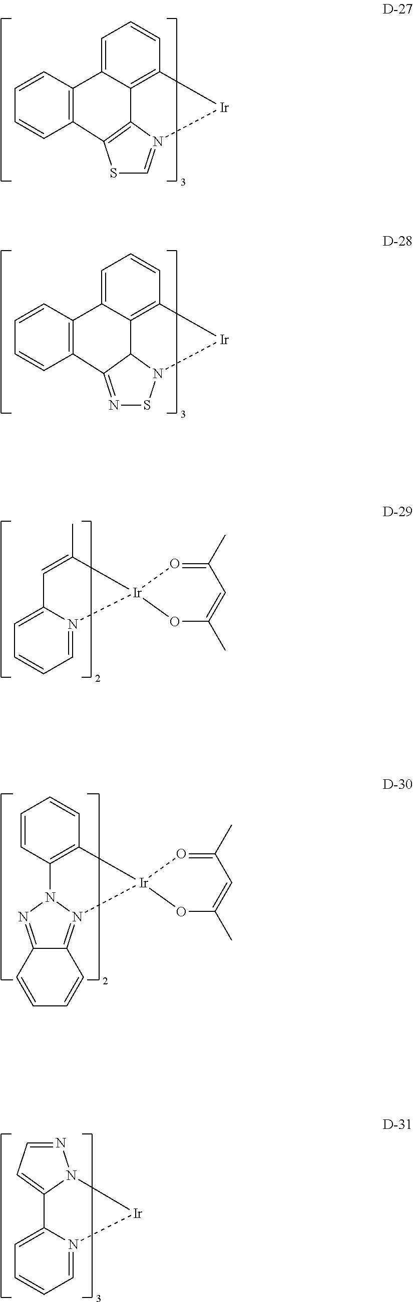

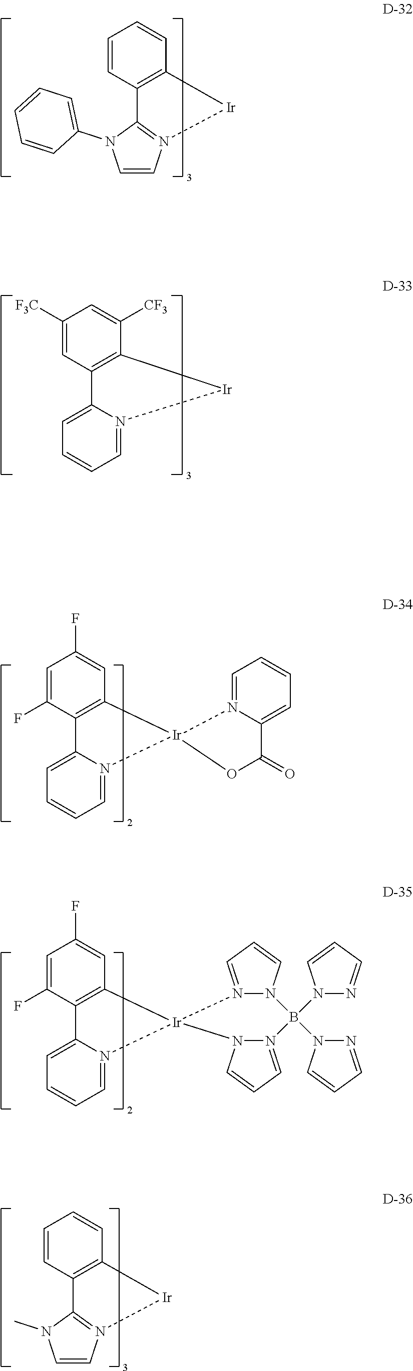

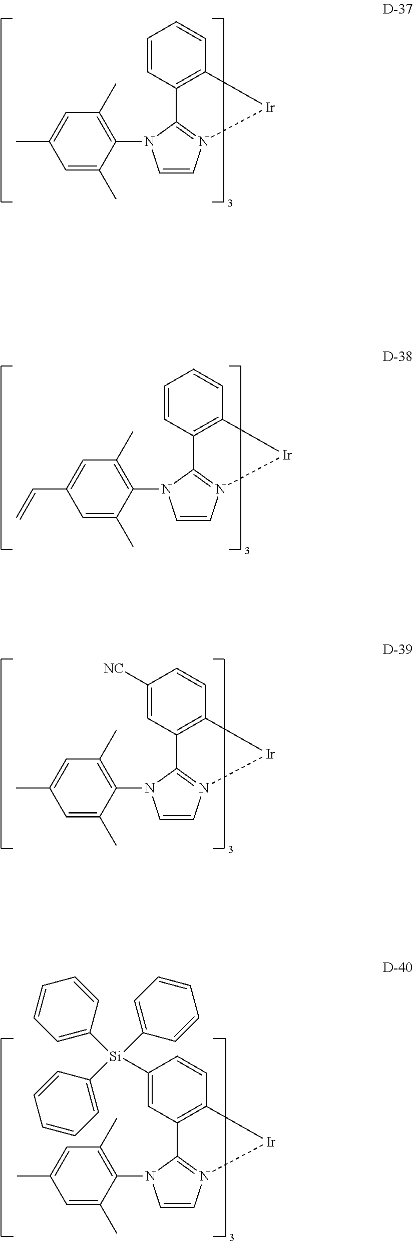

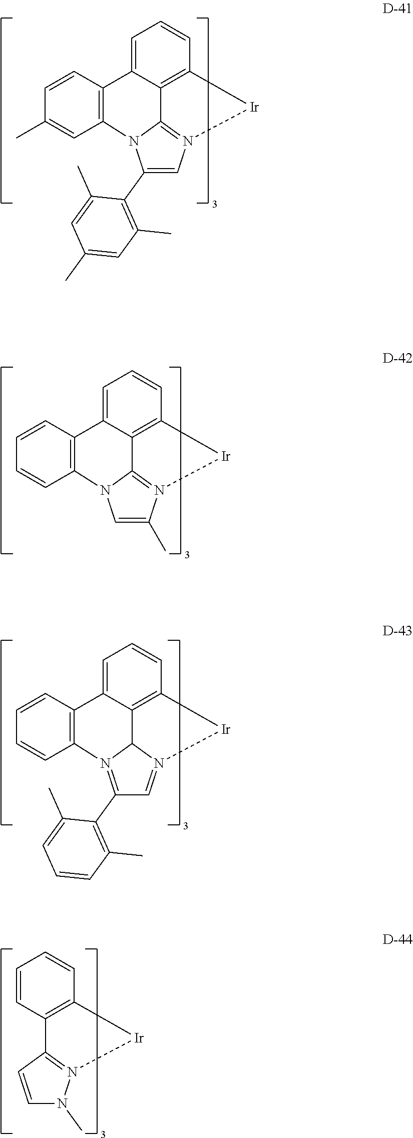

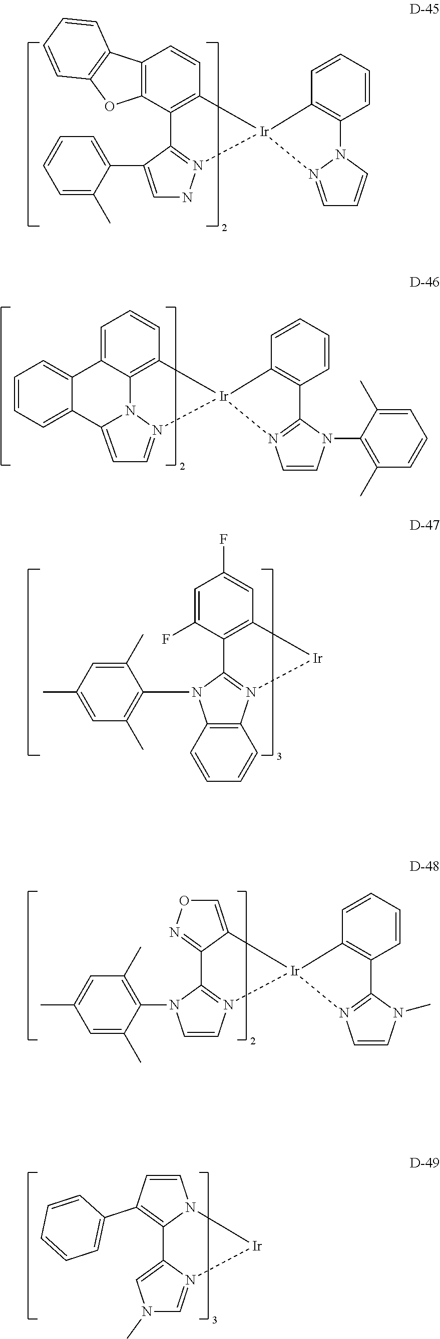

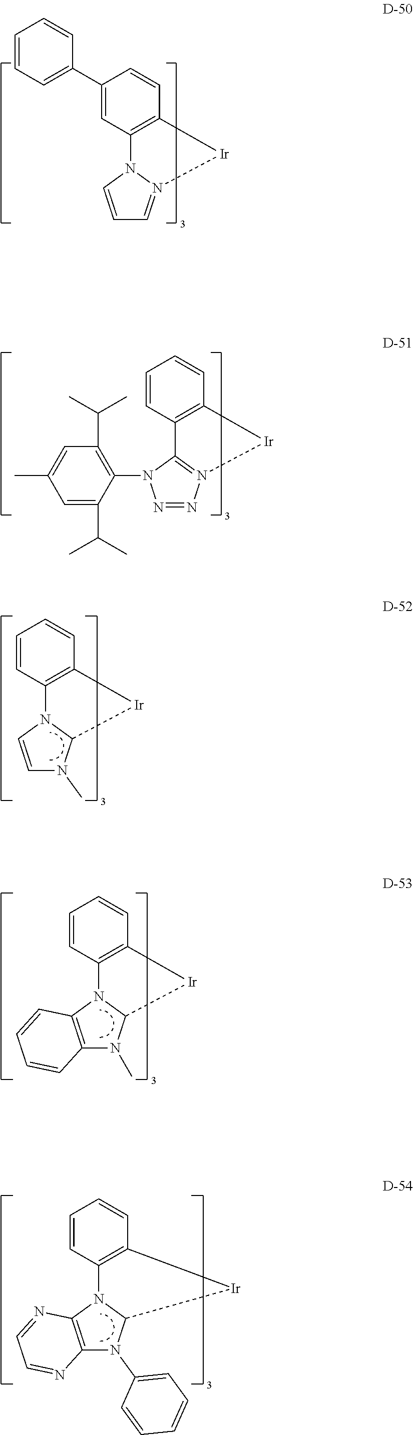

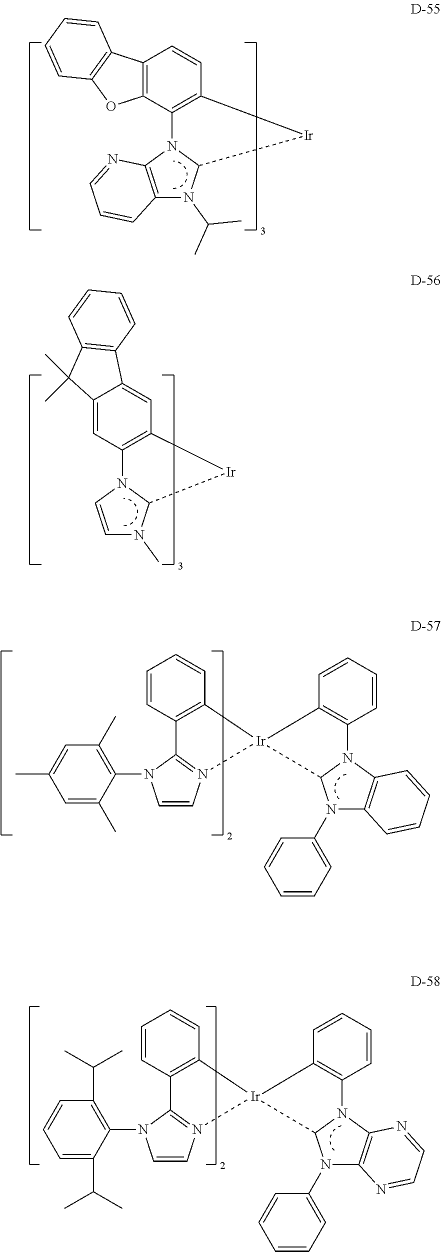

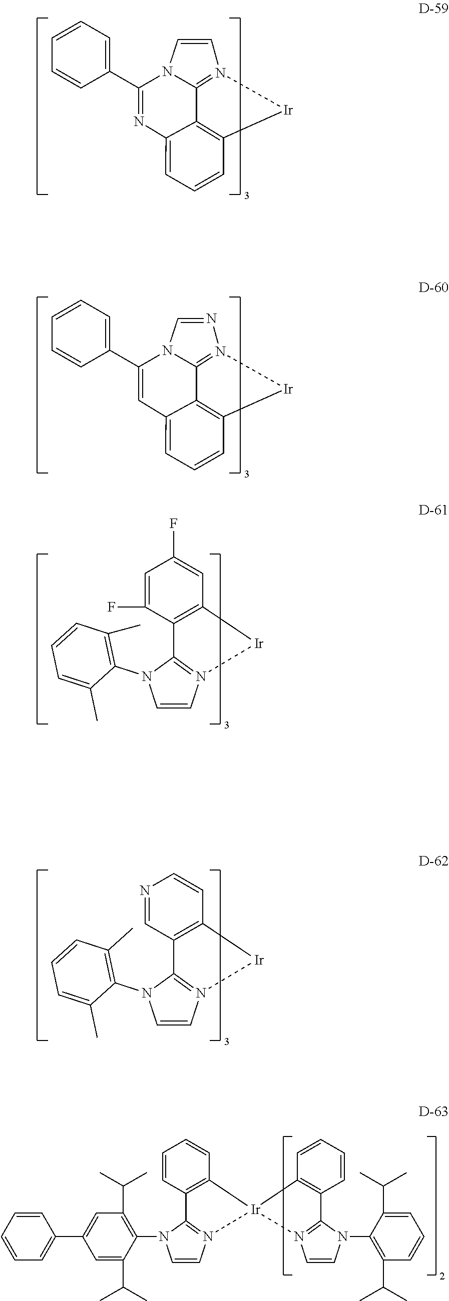

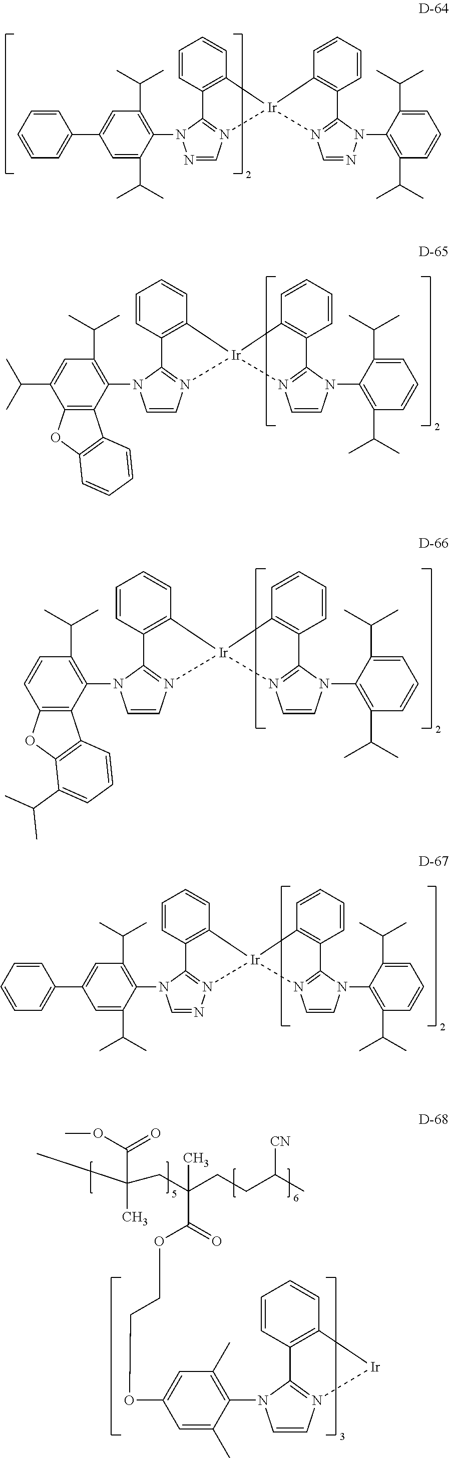

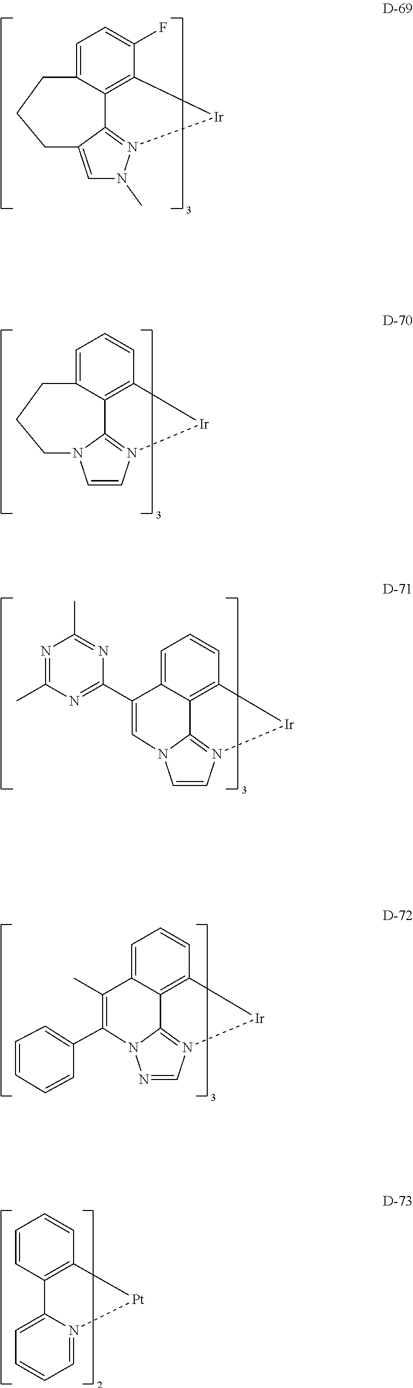

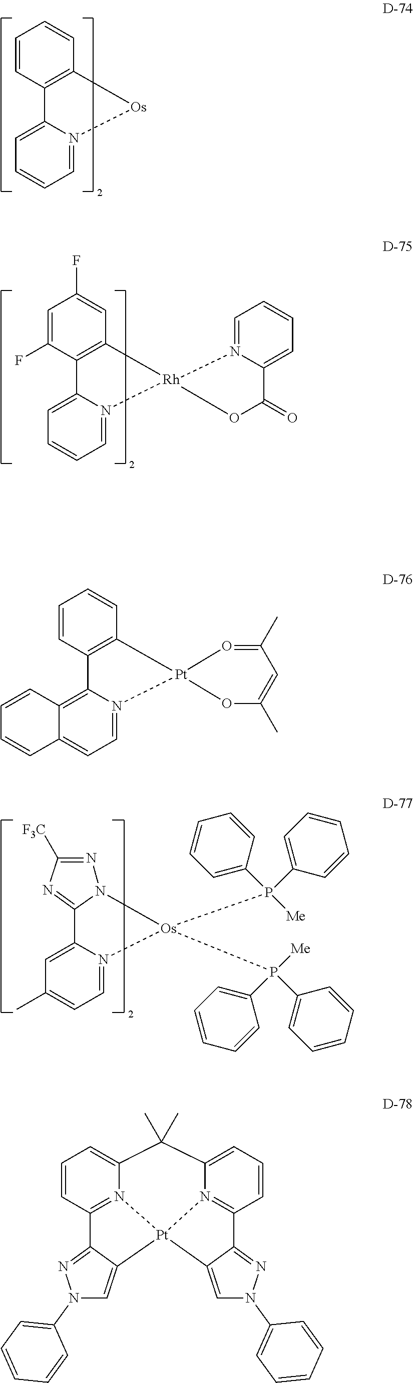

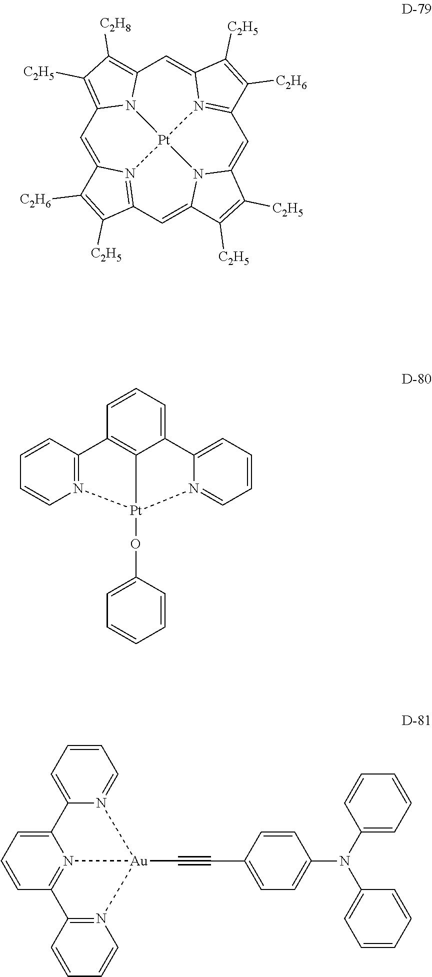

### (1-2. Fluorescence Emitting Dopant)

The fluorescence emitting dopant is a compound which is observed emission from an excited singlet state thereof. The compound is not limited as long as emission from an excited singlet state is observed.

As specific known fluorescence emitting dopants usable in the present invention, listed are compounds such as: an anthracene derivative, a pyrene derivative, a chrysene derivative, a fluoranthene derivative, a perylene derivative, a fluorene derivative, an arylacetylene derivative, a styrylarylene derivative, a styrylamine derivative, an arylamine derivative, a boron complex, a coumarin derivative, a pyran derivative, a cyanine derivative, a croconium derivative, a squarylium derivative, an oxobenzanthracene derivative, a fluorescein derivative, a rhodamine derivative, a pyrylium derivative, a perylene derivative, a polythiophene derivative, and a rare earth complex compound.

In addition, it may be used an emission dopant utilizing delayed fluorescence. Specific examples of utilizing delayed fluorescence are compounds described in: WO 2011/156793, JP-A No. 2011-213643, and JP-A No. 2010-93181. However, the present invention is not limited to them.

### (2. Host Compound)

A host compound is a compound which mainly plays a role of injecting or transporting a charge in a light emitting layer. In an organic EL element, an emission from the host compound itself is substantially not observed.

Preferably, it is a compound exhibiting a phosphorescent emission yield of less than 0.1 at a room temperature (25° C.), more preferably a compound exhibiting a phosphorescent emission yield of less than 0.01. Among the compounds incorporated in the light emitting layer, a mass ratio of the host compound in the light emitting layer is preferably at least 20%.

It is preferable that the excited energy level of the host compound is higher than the excited energy level of the dopant contained in the same layer.

Host compounds may be used singly or may be used in combination of two or more compounds. By using a plurality of host compounds, it is possible to adjust transfer of charge, thereby it is possible to achieve an organic EL element of high efficiency.

As a host compound in the light emitting layer, an organic EL element material of the present invention may be used. The material contains a compound represented by the above-described Formula (1).

A compound used in a well-known organic EL element may be used in combination of an organic EL element material of the present invention.

Examples of a compound that may be used in combination are: a carbazole derivative, a triarylamine derivative, an aromatic derivative, a nitrogen-containing heterocyclic compound, a thiophene derivative, a furan derivative, a compound having a basic skeleton of oligoarylene, a carboline derivative, and a diazacarbazole derivative (indicating a ring structure in which at least one of the carbon atoms constituting the carboline ring of the carboline derivative is replaced with a nitrogen atom).

As a known host compound that may be used in the present invention, preferably, it is a compound having a hole transporting ability or an electron transporting ability, as well as preventing elongation of an emission wavelength. In addition, from the viewpoint of stably driving an organic EL element at high temperature, it is preferable that a host compound has a high glass transition temperature (Tg) of 100° C. or more.

By using a plurality of host compounds, it is possible to adjust transfer of charge, thereby it is possible to achieve an organic EL element of high efficiency.

By using a plurality of compounds known as a phosphorescent dopant, it is possible to mix light of different color, thereby an arbitral emission color may be obtained.

A host compound used in a light emitting layer may be a low molecular weight compound, or a polymer having a recurring unit. Further, it may be a low molecular weight compound having a reactive group such as a vinyl group or an epoxy group (a polymerizable host). One or plurality of these compounds may be used.

As specific examples of a known host compound used in an organic EL element, the compounds described in the following documents are cited.

Japanese patent application publication (JP-A) Nos. 2001-257076, 2002-308855, 2001-313179, 2002-319491, 2001-357977, 2002-334786, 2002-8860, 2002-334787, 2002-15871, 2002-334788, 2002-43056, 2002-334789, 2002-75645, 2002-338579, 2002-105445, 2002-343568, 2002-141173, 2002-352957, 2002-203683, 2002-363227, 2002-231453, 2003-3165, 2002-234888, 2003-27048, 2002-255934, 2002-260861, 2002-280183, 2002-299060, 2002-302516, 2002-305083, 2002-305084 and 2002-308837.

It may be a low molecular weight compound, or a polymer having a recurring unit. Further, it may be a compound having a reactive group such as a vinyl group or an epoxy group.

### <Electron Transport Layer>

An electron transport layer is composed of a material that has a function of transporting electrons. An electron injection layer and a hole blocking layer are included in an electron transport layer in broad sense. The electron transport layer may have a monolayer or multilayer configuration.

An electron transport layer is required to have a function of transporting electrons injected from the cathode to a light emitting layer. Any known materials may be arbitrary selected and used for the constituting material of the electron transport layer.

Examples of a known material used for the electron transport layer include: a nitro-substituted fluorene derivative, a diphenylquinone derivative, a thiopyrane dioxide derivative, a polycyclic aromatic hydrocarbon such as a naphthaleneperylene, heterocyclic tetra carboxylic acid anhydride, carbodiimide, a fluorenylidene methane derivative, an anthraquinodimethane derivative, an anthrone derivative, an oxadiazole derivative, a carboline derivative, a derivative in which at least one of the carbon atoms constituting the carboline ring of the carboline derivative is replaced with a nitrogen atom, and a hexaazatriphenylene derivative.

In addition, a thiadiazole derivative in which the oxygen atom in the oxadiazole ring is replaced with a sulfur atom in the oxadiazole derivatives, and a quinoxaline derivative having a quinoxaline ring being electron attractive groups may also be used as a material for the electron transport layer. Polymer materials containing these materials as polymer chains or main chains may also be used.

Further, metal complexes having a ligand of a 8-quinolinol structure or dibnenzoquinolinol structure such as tris(8-quinolinol)aluminum (Alq₃), tris(5,7-dichloro-8-quinolinol)aluminum, tris(5,7-dibromo-8-quinolinol)aluminum, tris(2-methyl-8-quinolinol)aluminum, tris(5-methyl-8-quinolinol)aluminum and bis(8-quinolinol)zinc (Znq); and metal complexes in which a central metal of the aforesaid metal complexes is substituted by In, Mg, Cu, Ca, Sn, Ga or Pb, may be also utilized as an electron transport material.

Further, a metal-free or metal phthalocyanine, or a compound whose terminal is substituted by an alkyl group or a sulfonic acid group, may be preferably utilized as an electron transport material. An inorganic semiconductor such as an n-type Si and an n-type SiC may be also utilized as an electron transport material.

An electron transport layer is preferably formed in a thin film with an electron transport material by using a method such as a vacuum vapor deposition method and a wet method. Examples of a wet process include: a spin coating method, a cast method, a die coating method, a blade coating method, a roll coating method, an inkjet method, a printing method, a spray coating method, a curtain coating method, and a LB method (Langmuir Blodgett method).

The electron transport layer may have any thickness, and usually it has a thickness of about 5 to 5,000 nm, preferably in the range of 5 to 200 nm. The electron transport layer may have a single layer configuration composed of one or more of the materials described above. Further, it may be used by being doped with an n-type dopant of a metal compound such as a metal complex and a metal halide.

As an example of a known electron transport material that is preferably used in an electron transport layer of an organic EL element of the present invention, it may be cited compounds described in WO 2013/061850. However, the present invention is not limited to them.

### <Cathode>

As a cathode, a metal having a small work function (4 eV or less) (it is called as an electron injective metal), an alloy, a conductive compound and a mixture thereof are utilized as an electrode substance. Specific examples of the aforesaid electrode substance includes: sodium, sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, aluminum, and a rare earth metal.

Among them, with respect to an electron injection property and durability against oxidation, preferable are: a mixture of election injecting metal with a second metal which is stable metal having a work function larger than the electron injecting metal. Examples thereof are: a magnesium/silver mixture, a magnesium/aluminum mixture, a magnesium/indium mixture, an aluminum/aluminum oxide (Al₂O₃) mixture, a lithium/aluminum mixture and aluminum.

A cathode may be made by using these electrode substances with a method such as a vapor deposition method or a sputtering method to form a thin film. A sheet resistance of a cathode is preferably a few hundred Ω/□ or less. A layer thickness of the cathode is generally selected in the range of 10 nm to 5 μm, and preferably in the range of 50 to 200 nm.

In order to transmit emitted light, it is preferable that one of an anode and a cathode of an organic EL element is transparent or translucent for achieving an improved luminescence.

Further, after forming a layer of the aforesaid metal having a thickness of 1 to 20 nm on the cathode, it is possible to prepare a transparent or translucent cathode by providing with a conductive transparent material described in the description for the anode thereon. By applying this process, it is possible to produce an element in which both an anode and a cathode are transparent.

### <Injection Layer: Electron Injection Layer (Cathode Buffer Layer) and Hole Injection Layer>

An injection layer is installed when required. There are an electron injection layer and a hole injection layer. It may be arranged: between a anode and a light emitting layer or a hole transport layer; or between a cathode and a light emitting layer or an electron transport layer. An injection layer is a layer to decrease a driving voltage and to improve an emission luminance. An example of an injection layer is detailed in volume 2, chapter 2 “Electrode materials” (pp. 123-166) of “Organic EL Elements and Industrialization Front thereof (Nov. 30, 1998, published by N.T.S. Co. Ltd.)”. There are a hole injection layer (an anode buffer layer) and an electron injection layer (a cathode buffer layer).

An anode buffer layer (a hole injection layer) is detailed in JP-A Nos. 9-45479, 9-260062, and 8-288069. Specific examples are: a phthalocyanine buffer layer represented by copper phthalocyanine; a hexaazatriphenylene derivative buffer layer described in JP-A Nos. 2003-519432 and 2006-135145; a metal oxide buffer layer represented by vanadium oxide; an amorphous carbon buffer layer, a polymer buffer layer made of a conductive polymer such as polyaniline (or called as emeraldine) and polythiophene; an orthometalated complex represented by tris(2-phenylpyridine) iridium complex; and a triarylamine derivative.

An election injection layer is detailed in JP-A Nos. 6-325871, 9-17574, and 10-74586. Examples thereof include: a metal buffer layer represented by strontium and aluminum; an alkaline metal compound buffer layer represented by lithium fluoride and potassium fluoride; an alkaline earth metal compound buffer layer represented by magnesium fluoride and cesium fluoride; and a metal oxide buffer layer represented by aluminum oxide.

The above-described buffer layer (injection layer) is preferably to be thin film. The thickness thereof is preferably in the range of 0.1 nm to 5 μm, although it depend on the material.

### <Blocking Layer: Hole Blocking Layer and Electron Blocking Layer>

A blocking layer is installed when required beside the basic constituting layers of the organic compound thin films as described above. Examples thereof are hole block layers described in JP-A Nos. 11-204258, 11-204359, and in page 237 of “Organic EL Elements and Industrialization Front thereof (Nov. 30, 1998, published by N.T.S. Co. Ltd.)”.

The hole blocking layer has a function of an electron transport layer in a broad sense. The hole blocking layer is composed of a hole blocking material that will transport electrons but barely transport holes. Since the hole blocking layer transports electrons while blocking holes, the layer will enhance the opportunity of recombination of electrons and holes. The configuration of the electron transport layer may be used as a hole blocking layer. Preferably, the hole blocking layer is disposed adjacent to the luminous layer.

The hole blocking layer preferably contains a compound cited as a host compound. Examples thereof are: a carbazole derivative, a carboline derivative, and a diazacarbazole derivative (here, a diazacarbazole derivative is a compound in which at least one of the carbon atoms constituting the carboline ring of the carboline derivative is replaced with a nitrogen atom).

On the other hand, an electron blocking layer is a layer provided with a function of a hole transport layer in a broad meaning. Preferably, it contains a material having a function of transporting a hole, and having very small ability of transporting an electron. It can improve the recombination probability of an electron and a hole by blocking an electron while transporting a hole.

Further, a composition of a hole transport layer described later may be appropriately utilized as an electron blocking layer when required.

A thickness of a hole blocking layer or an electron blocking layer is preferably in the range of 3 to 100 nm, and more preferably, in the range of 5 to 30 nm.

### <Hole Transport Layer>

A hole transport layer is composed of a hole transport material that will transport holes. The hole injection layer and electron blocking layer are included in a hole transport layer in a broad sense. The hole transport layer may be provided in a single layer or two or more layers.

The hole transport layer may be composed of any organic or inorganic compound which will inject or transport holes or will block electrons. Examples of such materials include triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline copolymers, conductive polymers and oligomers, and thiophene oligomers.

Further, a hexaazatriphenylene derivative described in JP-A Nos. 2003-519432 and 2006-135145 may be also used for a hole transport material.

The hole transport material may be porphyrin compounds, tertiary arylamine compounds, and styrylamine compounds, besides the compounds described above. Particularly preferred are tertiary arylamine compounds.

Typical examples of the tertiary arylamine compound and styrylamine compounds include: N,N,N′,N′-tetraphenyl-4,4′-diaminophenyl, N,N′-diphenyl-N,N′-bis(3-methylphenyl)-(1,1′-biphenyl)-4,4′-diamine (TPD), 2,2-bis(4-di-p-tolylaminophenyl)propane, 1,1-bis(4-di-p-tolylaminophenyl)cyclohexane, N,N,N′,N′-tetra-p-tolyl-4,4′-diaminobiphenyl, 1,1-bis(4-di-p-tolylaminophenyl)-4-phenylcyclohexane, bis(4-dimethylamino-2-methylphenyl)phenylmethane, bis(4-di-p-tolylaminopnenyl)phenylmethane, N,N′-diphenyl-N,N′-di(4-methoxyphenyl)-4,4′-diaminobiphenyl, N,N,N′,N′-tetraphenyl-4,4′-diaminodiphenyl ether, 4,4′-bis(diphenylamino)quodriphenyl, N,N,N-tri(p-tolyl)amine, 4-(di-p-tolylamino)-4′-(4-(di-p-tolylamino)styryl)stilbene, 4-N,N-diphenylamino-(2-diphenylvinyl)benzene, 3-methoxy-4′-N,N-diphenylaminostyrylbenzene, and N-phenylcarbazole.

Further, there are cited: a compound having 2 condensed aromatic rings in the molecule as described in U.S. Pat. No. 5,061,569 such as 4,4′-bis[N-(1-naphtyl)-N-phenylamino]biphenyl (NPD); a compound having 3 triphenylamine units in a star burst type as described in JP-A No. 4-308688 such as 4,4′,4″-tris[N-(3-methylphenyl)-N-phenylamino] triphenylamine (MTDATA).

It may be used a polymer material in which these material are introduced in a polymer side chain or in a polymer main chain.

Further, an inorganic compound such as an p-type Si and an p-type SiC may be also utilized as a hole injection material or a hole transport material.

Further, it is possible to employ so-called p-type hole transport materials, as described in JP-A No. 11-251067, and J. Huang et al. reference (Applied Physics Letters 80 (2002), p. 139). In the present invention, these materials are preferably used from the viewpoint of obtaining more efficient light emitting element.

A thin film of the hole transport layer may be formed with the hole transport material by any known process, for example, vacuum evaporation, spin coating, casting, printing such as ink jetting, or a LB method.

The hole transport layer may have any thickness, usually a thickness of about 5 nm to 5 μm, preferably 5 to 200 nm. The hole transport layer may have a single layer configuration composed of one or more of the materials described above.

The hole transport layer may be doped with any dopant to enhance p characteristics. Such techniques are described, for example, in JP-A Nos. 4-297076, 2000-196140, and 2001-102175, and J. Appl. Phys., 95, 5773(2004).

A hole transport layer with enhanced p characteristics is preferably used because it enables to produce elements with low power consumption.

### <Anode>

As an anode of an organic EL element, a metal having a large work function (4 eV or more), an alloy, and a conductive compound and a mixture thereof are utilized as an electrode substance. Specific examples of an electrode substance are: metals such as Au, and an alloy thereof; transparent conductive materials such as CuI, indium tin oxide (ITO), SnO₂, and ZnO. Further, a material such as IDIXO (In₂O₃—ZnO), which can form an amorphous and transparent electrode, may also be used.

As for an anode, these electrode substances may be made into a thin layer by a method such as a vapor deposition method or a sputtering method; followed by making a pattern of a desired form by a photolithography method. Otherwise, in the case of requirement of pattern precision is not so severe (about 100 μm or more), a pattern may be formed through a mask of a desired form at the time of layer formation with a vapor deposition method or a sputtering method using the above-described material.

Alternatively, when a coatable substance such as an organic conductive compound is employed, it is possible to employ a wet film forming method such as a printing method or a coating method. When emitted light is taken out from the anode, the transmittance is preferably set to be 10% or more. A sheet resistance of a first electrode is preferably a few hundred Ω/□ or less.

Further, although a layer thickness of the anode depends on a material, it is generally selected in the range of 10 nm to 1 μm, and preferably in the range of 10 to 200 nm.

### <Support Substrate>

A support substrate which may be used for an organic EL element of the present invention is not specifically limited with respect to types of such as glass and plastics. Hereafter, the support substrate may be also called as substrate body, substrate, substrate substance, or support. They me be transparent or opaque. However, a transparent support substrate is preferable when the emitting light is taken from the side of the support substrate. Support substrates preferably utilized includes such as glass, quartz and transparent resin film. A specifically preferable support substrate is a resin film capable of providing an organic EL element with a flexible property.

Examples of a resin film include: polyesters such as polyethylene terephthalate (PET) and polyethylene naphthalate (PEN), polyethylene, polypropylene, cellophane, cellulose esters and their derivatives such as cellulose diacetate, cellulose triacetate (TAC), cellulose acetate butyrate, cellulose acetate propionate (CAP), cellulose acetate phthalate, and cellulose nitrate, polyvinylidene chloride, polyvinyl alcohol, polyethylene vinyl alcohol, syndiotactic polystyrene, polycarbonate, norbornene resin, polymethyl pentene, polyether ketone, polyimide, polyether sulfone (PES), polyphenylene sulfide, polysulfones, polyether imide, polyether ketone imide, polyamide, fluororesin, Nylon, polymethyl methacrylate, acrylic resin, polyarylates and cycloolefin resins such as ARTON (trade name, made by JSR Co. Ltd.) and APEL (trade name, made by Mitsui Chemicals, Inc.).

On the surface of a resin film, it may be formed a film incorporating an inorganic or an organic compound or a hybrid film incorporating both compounds. Barrier films are preferred at a water vapor permeability of 0.01 g/m².24 h or less (at 25±0.5° C., and 90±2% RH) determined based on JIS K 7129-1992. Further, high barrier films are preferred to have an oxygen permeability of 1×10⁻³ ml/m²·24 h. atm or less determined based on JIS K 7126-1987, and a water vapor permeability of 1×10⁻⁵ g/m²·24 h or less.

As materials forming a barrier film, employed may be those which retard penetration of moisture and oxygen, which deteriorate the element. For example, it is possible to employ silicon oxide, silicon dioxide, and silicon nitride. Further, in order to improve the brittleness of the aforesaid film, it is more preferable to achieve a laminated layer structure of inorganic layers and organic layers. The laminating order of the inorganic layer and the organic layer is not particularly limited, but it is preferable that both are alternatively laminated a plurality of times.

Barrier film forming methods are not particularly limited, and examples of employable methods include a vacuum deposition method, a sputtering method, a reactive sputtering method, a molecular beam epitaxy method, a cluster ion beam method, an ion plating method, a plasma polymerization method, a plasma CVD method, a laser CVD method, a thermal CVD method, and a coating method. Of these, specifically preferred is a method employing an atmospheric pressure plasma polymerization method, described in JP-A No. 2004-68143.

Examples of opaque support substrates include metal plates such aluminum or stainless steel films, opaque resin substrates, and ceramic substrates.

The external taking out quantum efficiency of light emitted by the organic EL element of the present invention is preferably at least 1% at a room temperature, but is more preferably at least 5%.

External taking out quantum efficiency (%)=(Number of photons emitted by the organic EL element to the exterior/Number of electrons fed to organic EL element)×100.

Further, it may be used simultaneously a color hue improving filter such as a color filter, or it may be used simultaneously a color conversion filter which convert emitted light color from the organic EL element to multicolor by employing fluorescent materials. When a color conversion filter is used, it is preferable that a maximum emission wavelength (λmax) of an organic EL element is 480 nm or less.

### [Production Method of Organic EL Element]

As an example of a production method of an organic EL element, it will be described a production method of an organic EL element having the following configuration.

Anode/hole injection layer/hole transport layer/light emitting layer/hole blocking layer/electron transport layer/cathode buffer layer (electron injection layer)/cathode

First, an anode is produced on a suitable substrate by forming a thin film made of an anode material with a thickness of 1 μm or more, preferably, 10 to 200 nm.

Then, on this are formed thin films containing organic compounds for element constituting materials: a hole injection layer, a hole transport layer, a light emitting layer, a hole blocking layer, an electron transport layer, and a cathode buffer layer.

As a method of forming a thin film, it may be used a vacuum deposition method or a wet method (it may be called as a wet process).

Examples of a wet process include: a spin coating method, a cast method, an inkjet method, a printing method, a die coating method, a blade coating method, a roll coating method, a spray coating method, a curtain coating method, and a LB method. From the viewpoint of getting a uniform thin layer with high productivity, preferable are method highly appropriate to a roll-to-roll method such as a die coating method, a roll coating method, an inkjet method, and a spray coating method. A different coating method may be used for a different layer.

Examples of a liquid medium to dissolve or to disperse an organic EL material such as an emission dopant used in the present invention include: ketones such as methyl ethyl ketone and cyclohexanone; aliphatic esters such as ethyl acetate; halogenated hydrocarbons such as dichlorobenzene; aromatic hydrocarbons such as toluene, xylene, mesitylene, and cyclohexylbenzene; aliphatic hydrocarbons such as cyclohexane, decalin, and dodecane; organic solvents such as dimethylformamide (DMF) and DMSO.

These will be dispersed with a dispersion method such as an ultrasonic dispersion method, a high shearing dispersion method and a media dispersion method.

After forming these layers, it is formed a thin film made of a cathode forming material is formed with a thickness of 1 μm or less, preferably, 50 to 200 nm. Thus, a cathode is formed on these layers to produce a required organic EL element.

It is possible to produce an organic EL element with a reversed order of the layer production to form: a cathode, a cathode buffer layer, an electron transport layer, a hole blocking layer, a light emitting layer, a hole transport layer, a hole injection layer, and an anode.

Formation of organic layers of the present invention is preferably continuously carried out from a hole injection layer to a cathode with one time vacuuming. It may be taken out on the way, and a different layer forming method may be employed. In that case, the operation is preferably done under a dry inert gas atmosphere.

### [Sealing]

As sealing means employed in the present invention, listed may be, for example, a method in which sealing members, electrodes, and a supporting substrate are subjected to adhesion via adhesives. The sealing members may be arranged to cover the display region of an organic EL element, and may be a concave plate or a flat plate. Neither transparency nor electrical insulation is limited.

Specifically listed are: glass plates, polymer plate-films, and metal plate-films. Specifically, it is possible to list, as glass plates, soda-lime glass, barium-strontium containing glass, lead glass, aluminosilicate glass, borosilicate glass, barium borosilicate glass, and quartz. Further, listed as polymer plates maybe polycarbonate, acryl, polyethylene terephthalate, polyether sulfide, and polysulfone. As a metal plate, listed are those composed of at least one metal selected from the group consisting of stainless steel, iron, copper, aluminum magnesium, nickel, zinc, chromium, titanium, molybdenum, silicon, germanium, and tantalum, or alloys thereof.

In the present invention, since it is possible to achieve a thin organic EL element, it is preferable to employ a polymer film or a metal film. Further, it is preferable that the polymer film has an oxygen permeability of 1×10⁻³ ml/m²·24 h or less determined by the method based on JIS K 7126-1987, and a water vapor permeability of 1×10⁻³ g/m²·24 h or less (at 25±0.5° C., and 90±2% RH) or less determined by the method based on JIS K 7129-1992.

Conversion of the sealing member into concave is carried out employing a sand blast process or a chemical etching process.

In practice, as adhesives, listed may be photo-curing and heat-curing types having a reactive vinyl group of acrylic acid based oligomers and methacrylic acid, as well as moisture curing types such as 2-cyanoacrylates. Further listed may be thermal and chemical curing types (mixtures of two liquids) such as epoxy based ones. Still further listed may be hot-melt type polyamides, polyesters, and polyolefins. Yet further listed may be cationically curable type UV curable epoxy resin adhesives.

In addition, since an organic EL element is occasionally deteriorated via a thermal process, those are preferred which enable adhesion and curing between a room temperature and 80° C. Further, desiccating agents may be dispersed into the aforesaid adhesives. Adhesives may be applied onto sealing portions via a commercial dispenser or printed on the same in the same manner as screen printing.

Further, it is appropriate that on the outside of the aforesaid electrode which interposes the organic layer and faces the support substrate, the aforesaid electrode and organic layer are covered, and in the form of contact with the support substrate, inorganic and organic material layers are formed as a sealing film. In this case, as materials forming the aforesaid film may be those which exhibit functions to retard penetration of moisture or oxygen which results in deterioration. For example, it is possible to employ silicon oxide, silicon dioxide, and silicon nitride.

Still further, in order to improve brittleness of the aforesaid film, it is preferable that a laminated layer structure is formed, which is composed of these inorganic layers and layers composed of organic materials. Methods to form these films are not particularly limited. It is possible to employ, for example, a vacuum deposition method, a sputtering method, a reactive sputtering method, a molecular beam epitaxy method, a cluster ion beam method, an ion plating method, a plasma polymerization method, an atmospheric pressure plasma polymerization method, a plasma CVD method, a thermal CVD method, and a coating method.

It is preferable to inject a gas phase and a liquid phase material of inert gases such as nitrogen or argon, and inactive liquids such as fluorinated hydrocarbon or silicone oil into the space between the space formed with the sealing member and the display region of the organic EL element. Further, it is possible to form vacuum in the space. Still further, it is possible to enclose hygroscopic compounds in the interior of the space.

Examples of hygroscopic compounds include: metal oxides (for example, sodium oxide, potassium oxide, calcium oxide, barium oxide, magnesium oxide, and aluminum oxide); sulfates (for example, sodium sulfate, calcium sulfate, magnesium sulfate, and cobalt sulfate); metal halides (for example, calcium chloride, magnesium chloride, cesium fluoride, tantalum fluoride, cerium bromide, magnesium bromide, barium iodide, and magnesium iodide); perchlorates (for example, barium perchlorate and magnesium perchlorate). In sulfates, metal halides, and perchlorates, suitably employed are anhydrides. For sulfate salts, metal halides and perchlorates, suitably used are anhydrous salts.

### [Protective Film and Protective Plate]

On the aforesaid sealing film which interposes the organic layer and faces the support substrate or on the outside of the aforesaid sealing film, a protective or a protective plate may be arranged to enhance the mechanical strength of the element. Specifically, when sealing is achieved via the aforesaid sealing film, the resulting mechanical strength is not always high enough, whereby it is preferable to arrange the protective film or the protective plate described above. Usable materials for these include glass plates, polymer plate-films, and metal plate-films which are similar to those employed for the aforesaid sealing. However, in terms of light weight and decrease in thickness, it is preferable to employ a polymer film.

### [Light Extraction]

It is generally known that an organic EL element emits light in the interior of the layer exhibiting the refractive index (being about 1.7 to 2.1) which is greater than that of air, whereby only about 15% to 20% of light generated in the light emitting layer is extracted. This is due to the fact that light incident to an interface (being an interlace of a transparent substrate to air) at an angle of 0 which is at least critical angle is not extracted to the exterior of the element due to the resulting total reflection, or light is totally reflected between the transparent electrode or the light emitting layer and the transparent substrate, and light is guided via the transparent electrode or the light emitting layer, whereby light escapes in the direction, of the element side surface.

Means to enhance the efficiency of the aforesaid light extraction include, for example: a method in which roughness is formed on the surface of a transparent substrate, whereby total reflection is minimized at the interface of the transparent substrate to air (U.S. Pat. No. 4,774,435), a method in which efficiency is enhanced in such a manner that a substrate results in light collection (JP-A No. 63-314795), a method in which a reflection surface is formed on the side of the element (JP-A No. 1-220394), a method in which a flat layer of a middle refractive index is introduced between the substrate and the light emitting body and an antireflection film is formed (JP-A No. 62-172691), a method in which a flat layer of a refractive index which is equal to or less than the substrate is introduced between the substrate and the light emitting body (JP-A No. 2001-202827), and a method in which a diffraction grating is formed between the substrate and any of the layers such as the transparent electrode layer or the light emitting layer (including between the substrate and the outside) (JP-A No. 11-283751).

In the present invention, it is possible to employ these methods while combined with the organic EL element of the present invention. Of these, it is possible to appropriately employ the method in which a flat layer of a refractive index which is equal to or less than the substrate is introduced between the substrate and the light emitting body and the method in which a diffraction grating is formed between any layers of a substrate, and a transparent electrode layer and a light emitting layer (including between the substrate and the outside space).

By combining these means, the present invention enables the production of elements which exhibit higher luminance or excel in durability.

When a low refractive index medium having a thickness, greater than the wavelength of light is formed between the transparent electrode and the transparent substrate, the extraction efficiency of light emitted from the transparent electrode to the exterior increases as the refractive index of the medium decreases.

As materials of the low refractive index layer, listed are, for example, aerogel, porous silica, magnesium fluoride, and fluorine based polymers. Since the refractive index of the transparent substrate is commonly about 1.5 to 1.7, the refractive index of the low refractive index layer is preferably approximately 1.5 or less. More preferably, it is 1.35 or less.

Further, thickness of the low refractive index medium is preferably at least two times of the wavelength in the medium. The reason is that, when the thickness of the low refractive index medium reaches nearly the wavelength of light so that electromagnetic waves escaped via evanescent enter into the substrate, effects of the low refractive index layer are lowered.

The method in which the interface which results in total reflection or a diffraction grating is introduced in any of the media is characterized, in that light extraction efficiency is significantly enhanced. The above method works as follows. By utilizing properties of the diffraction grating capable of changing the light direction to the specific direction different from diffraction via so-called Bragg diffraction such as primary diffraction or secondary diffraction of the diffraction grating, of light emitted from the light entitling layer, light, which is not emitted to the exterior due to total reflection between layers, is diffracted via introduction of a diffraction grating between any layers or in a medium (in the transparent substrate and the transparent electrode) so that light is extracted to the exterior.

It is preferable that the introduced diffraction grating exhibits a two-dimensional periodic refractive, index. The reason is as follows. Since light emitted in the light emitting layer is randomly generated to all directions, in a common one-dimensional diffraction grating exhibiting a periodic refractive index distribution only in a certain direction, light which travels to the specific direction is only diffracted, whereby light extraction efficiency is not sufficiently enhanced.

However, by changing the refractive index distribution to a two-dimensional one, light, which travels to all directions, is diffracted, whereby the light extraction efficiency is enhanced.

A position to introduce a diffraction grating may be between any layers or in a medium (in a transparent substrate or a transparent electrode). However, a position near the organic light emitting layer, where light is generated, is preferable. In this case, the cycle of the diffraction grating is preferably from about ½ to 3 times of the wavelength of light in the medium. The preferable arrangement of the diffraction grating is such that the arrangement is two-dimensionally repeated in the form of a square lattice, a triangular lattice, or a honeycomb lattice.

### [Light Collection Sheet]

Via a process to arrange a structure such as a micro-lens array shape on the light extraction side of the organic EL element of the present invention or via combination with a so-called light collection sheet, light is collected in the specific direction such as the front direction with respect to the light emitting element surface, whereby it is possible to enhance luminance in the specific direction.

In an example of the micro-lens array, square pyramids to realize a side length of 30 μm and an apex angle of 90 degrees are two-dimensionally arranged on the light extraction side of the substrate. The side length is preferably 10 to 100 μm. When it is less than the lower limit, coloration occurs due to generation of diffraction effects, while when it exceeds the upper limit, the thickness increases undesirably.

It is possible to employ, as a light collection sheet, for example, one which is put into practical use in the LED backlight of liquid crystal display devices. It is possible to employ, as such a sheet, for example, the luminance enhancing film (BEF), produced by Sumitomo 3M Limited. As shapes of a prism sheet employed may be, for example, A shaped stripes of an apex angle of 90 degrees and a pitch of 50 μm formed on a base material, a shape in which the apex angle is rounded, a shape in which the pitch is randomly changed, and other shapes.

Further, in order to control the light radiation angle from the light emitting element, simultaneously employed may be a light diffusion plate-film. For example, it is possible to employ the diffusion film (LIGHT-UP), produced by Kimoto Co., Ltd.

### [Applications]

It is possible to employ the organic EL element of the present invention as display devices, displays, and various types of light emitting sources.

Examples of light emitting sources include: lighting apparatuses (home lighting and car lighting), clocks, backlights for liquid crystals, sign advertisements, signals, light sources of light memory media, light sources of electrophotographic copiers, light sources of light communication processors, and light sources of light sensors. The present invention is not limited to them. It is especially effectively employed as a backlight of a liquid crystal display device and a lighting source.

If needed, the organic EL element of the present invention may undergo patterning via a metal mask or an ink-jet printing method during film formation. When the patterning is carried out, only an electrode may undergo patterning, an electrode and a light emitting layer may undergo patterning, or all element layers may undergo patterning. During preparation of the element, it is possible to employ conventional methods.

Color of light emitted by an organic EL element or a compound of the present invention is specified as follows. In FIG. 4.16 on page 108 of “Shinpen Shikisai Kagaku Handbook (New Edition Color Science Handbook)” (edited by The Color Science Association of Japan, Tokyo Daigaku Shuppan Kai, 1985), values determined via a spectroradiometer CS-1000 (produced by Konica Minolta, Inc.) are applied to the CIE chromaticity coordinate, whereby the color is specified.

It is preferable that “white” in the organic EL element of the present invention shows chromaticity in the CIE 1931 Color Specification System at 1,000 cd/m² in the region of x=0.39±0.09 and y=0.38±0.08, when measurement is done to 2-degree viewing angle front luminance via the aforesaid method.

### [Display Device]

A display device provided with an organic EL element of the present invention may emit a single color or multiple colors. Here, it will be described a multiple color display device.

In case of a multiple color display device, a shadow mask is placed during the formation of a light emitting layer, and a layer is formed as a whole with a vapor deposition method, a cast method, a spin coating method, an inkjet method, and a printing method.

When patterning is done only to the light emitting layer, although the coating method is not limited in particular, preferable methods are a vapor deposition method, an inkjet method, a spin coating method, and a printing method.

A constitution of an organic EL element provided for a display device is selected from the above-described examples of an organic EL element according to the necessity.

The production method of an organic EL element is described as an embodiment of a production method of the above-described organic EL element.

When a direct-current voltage is applied to the produced multiple color display device, light emission can be observed by applying voltage of 2 o 40 V by setting the anode to have a plus (+) polarity, and the cathode to have a minus (−) polarity. When the voltage is applied to the device with reverse polarities, an electric current does not pass and light emission does not occur. Further, when an alternating-current voltage is applied to the device, light emission occurs only when the anode has a plus (+) polarity and the cathode has a minus (−) polarity. In addition, an arbitrary wave shape may be used for applying alternating-current.

The multiple color display device may be used for a display device, a display, and a variety of light emitting sources. In a display device or a display, a full color display is possible by using 3 kinds of organic EL elements emitting blue, red and green.

Examples of a display device or a display are: a television set, a personal computer, a mobile device, an AV device, a character broadcast display, and an information display in a car. Specifically, it may be used for a display device reproducing a still image or a moving image. When it is used for a display device reproducing a moving image, the driving mode may be any one of a passive-matrix mode and an active-matrix mode.

Examples of light emitting sources include: home lighting, car lighting, backlights for clocks and liquid crystals, sign advertisements, signals, light sources of light memory media, light sources of electrophotographic copiers, light sources of light communication processors, and light sources of light sensors. The present invention is not limited to them.

In the following, an example of a display device provided with an organic EL element of the present invention will be described by referring to drawings.

FIG. 1 is a schematic drawing illustrating an example of a display device composed of an organic EL element. Display of image information is carried out by light emission of an organic EL element. For example, it is a schematic drawing of a display of a cell-phone.

A display 1 is constituted of a display section A having plural number of pixels, a control section B which performs image scanning of the display section A based on image information, and a wiring section C electrically connecting the display section A and the control section B.

The control section B, which is electrically connected to the display section A via the wiring section C, sends a scanning signal and an image data signal to plural number of pixels based on image information from the outside and pixels of each scanning line successively emit depending on the image data signal by a scanning signal to perform image scanning, whereby image information is displayed on the display section A.

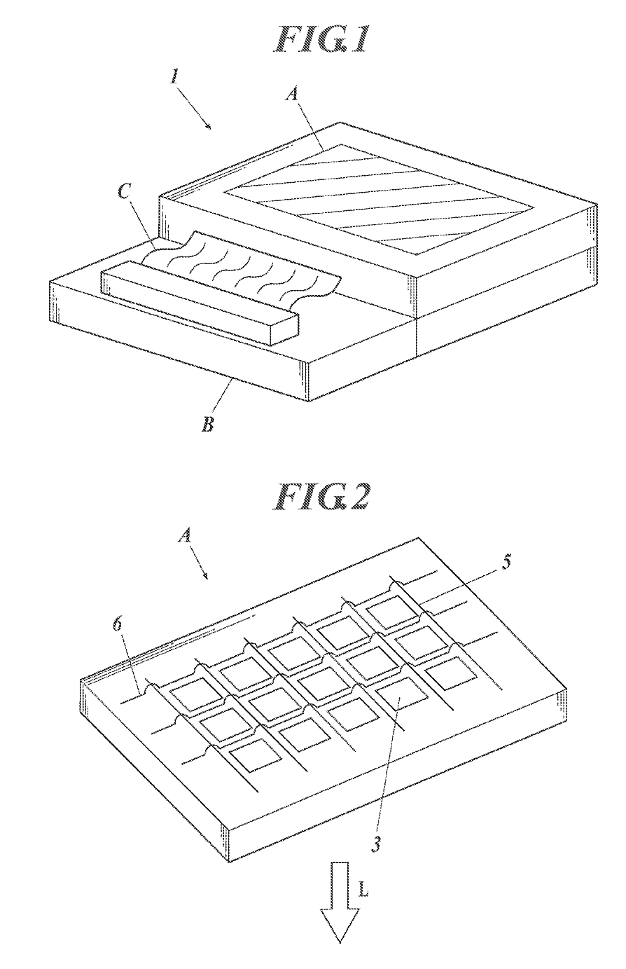

FIG. 2 is a schematic drawing of the display section A based on an active matrix mode.

The display section A is provided with the wiring section C, which contains plural scanning lines 5 and data lines 6, and plural pixels 3 on a substrate. Primary part materials of the display section A will be explained in the following.

In FIG. 2, shown is the case that light emitted by the pixel 3 is taken out along the white allow (downward).

The scanning lines 5 and the plural data lines 6 each are comprised of a conductive material, and the scanning lines 5 and the data lines 6 are perpendicular in a grid form and are connected to pixels 3 at the right-angled crossing points (details are not shown in the drawing).

The pixel 3 receives an image data from the data line 6 when a scanning signal is applied from the scanning line 5 and emits according to the received image data.

Full-color display is possible by appropriately arranging pixels having an emission color in a red region, pixels in a green region and pixels in a blue region, side by side on the same substrate.

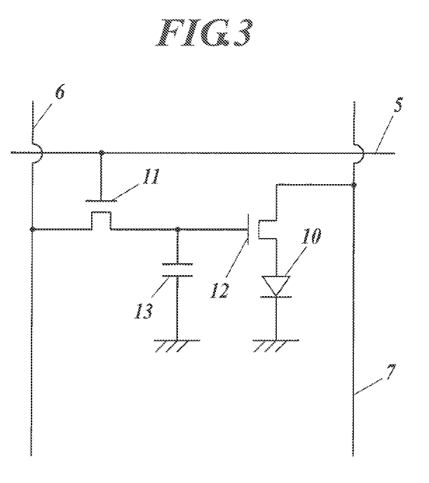

Next, an emission process of a pixel will be explained. FIG. 3 is a schematic drawing of a pixel.

A pixel is equipped with an organic EL element 10, a switching transistor 11, an operating transistor 12 and a capacitor 13. Red, green and blue emitting organic EL elements are utilized as the organic EL element 10 for plural pixels, and full-color display device is possible by arranging these side by side on the same substrate.

In FIG. 3, an image data signal is applied on the drain of the switching transistor 11 via the data line 6 from the control section B. Then when a scanning signal is applied on the gate of the switching transistor 11 via the scanning line 5 from control section B, operation of switching transistor is on to transmit the image data signal applied on the drain to the gates of the capacitor 13 and the operating transistor 12.

The operating transistor 12 is on, simultaneously with the capacitor 13 being charged depending on the potential of an image data signal, by transmission of an image data signal. In the operating transistor 12, the drain is connected to an electric source line 7 and the source is connected to the electrode of the organic EL element 10, and an electric current is supplied from the electric source line 7 to the organic EL element 10 depending on the potential of an image data applied on the gate.

When a scanning signal is transferred to the next scanning line 5 by successive scanning of the control section B, operation of the switching transistor 11 is off.

However, since the condenser 13 keeps the charged potential of an image data signal even when operation of the switching transistor 11 is off, operation of the operating transistor 12 is kept on to continue emission of the organic EL element 10 until the next scanning signal is applied.

When the next scanning signal is applied by successive scanning, the operating transistor 12 operates depending on the potential of an image data signal synchronized to the scanning signal and the organic EL element 10 emits light.

That is, emission of each organic EL element 10 of the plural pixels 3 is performed by providing the switching transistor 11 and the operating transistor 12 against each organic EL element 10 of plural pixels 3. Such an emission method is called as an active matrix mode.

Herein, emission of the organic EL element 10 may be either emission of plural gradations based on a multiple-valued image data signal having plural number of gradation potentials or on and off of a predetermined emission quantity based on a binary image data signal. Further, potential hold of the capacitor 13 may be either continuously maintained until the next scanning signal application or discharged immediately before the next scanning signal application.

In the present invention, emission operation is not necessarily limited to the above-described active matrix mode but may be a passive matrix mode in which organic EL element is emitted based on a data signal only when a scanning signal is scanned.

FIG. 4 is a schematic drawing of a display device based on a passive matrix mode. In FIG. 10, plural number of scanning lines 5 and plural number of image data lines 6 are arranged grid-wise, opposing to each other and sandwiching the pixels 3.

When a scanning signal of the scanning line 5 is applied by successive scanning, the pixel 3 connected to the scanning line 5 applied with the signal emits depending on an image data signal.

Since the pixel 3 is provided with no active element in a passive matrix mode, decrease of manufacturing cost is possible.

By employing the organic EL element of the present invention, it was possible to obtain a display device having improved emission efficiency.

### [Light Emitting Device]

An organic EL element of the present invention is preferably used for a light emitting device.

An organic EL element of the present invention may be provided with a rasonator structure. The intended uses of the organic EL element provided with a rasonator structure are: a light source of a light memory media, a light source of an electrophotographic copier, a light source of a light communication processor, and a light sources of a light sensor, however, it is not limited to them. It may be used for the above-described purposes by making to emit a laser.

Further, an organic EL element of the present invention may be used for a kind of lamp such as for illumination or exposure. It may be used for a projection device for projecting an image, or may be used for a display device to directly observe a still image or a moving image thereon.

The driving mode used for a display device of a moving image reproduction may be any one of a passive matrix mode and an active matrix mode. By employing two or more kinds of organic EL elements of the present invention emitting a different emission color, it can produce a full color display device.

In addition, a fluorescent compound of the present invention may be applicable to an organic EL element substantially emitting white light as a light emitting device. For example, when a plurality of light emitting materials are employed, white light can be obtained by mixing colors of a plurality of emission colors. As a combination of the plurality of emission colors, it may be a combination of red, green and blue having emission maximum wavelength of three primary colors, or it may be a combination of colors having two emission maximum wavelength making use of the relationship of two complementary colors of blue and yellow, or blue-green and orange.

A production method of an organic EL element of the present invention is done by placing a mask only during formation of a light emitting layer, a hole transport layer and an electron transport layer. It can be produced by coating with a mask to make simple arrangement. Since other layers are common, there is no need of pattering with a mask. For example, it can produce an electrode uniformly with a vapor deposition method, a cast method, a spin coating method, an inkjet method, and a printing method. The production yield will be improved.

By using these methods, it is possible to produce a white organic EL element in which a plurality of light emitting elements are arranged in parallel to form an array state. The element itself emits white light.

### One Embodiment of Light Emitting Device of the Present Invention

One embodiment of light emitting devices of the present Invention provided with an organic EL element of the present invention will be described.

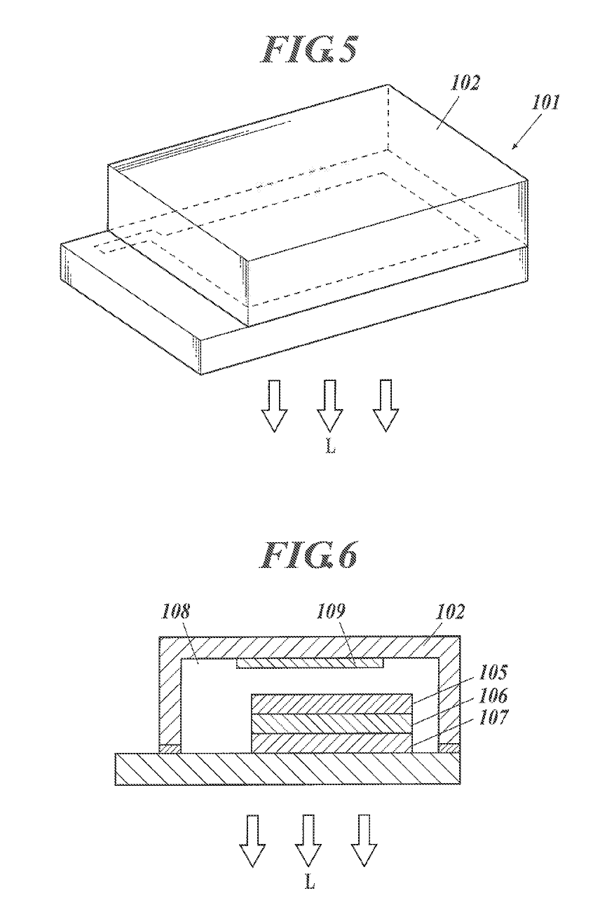

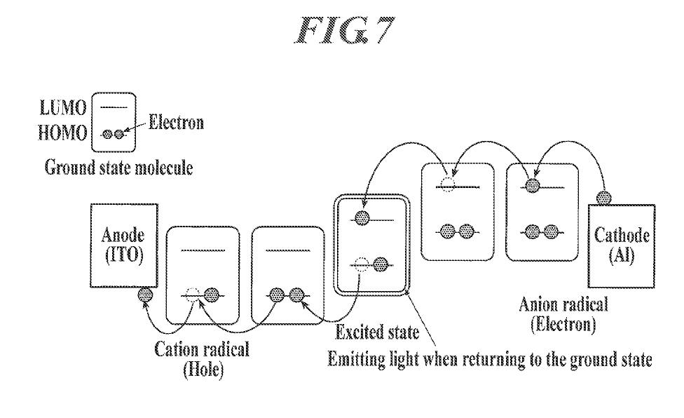

The non-light emitting surface of the organic EL element of the present invention was covered with a glass case, and a 300 μm thick glass substrate was employed as a sealing substrate. An epoxy based light curable type adhesive (LUXTRACK LC0629B produced by Toagosei Co., Ltd.) was employed in the periphery as a sealing material. The resulting one was superimposed on the aforesaid cathode to be brought into close contact with the aforesaid transparent support substrate, and curing and sealing were carried out via exposure of UV radiation onto the glass substrate side, whereby the light emitting device shown in FIG. 5 and FIG. 6, was formed.

FIG. 5 is a schematic view of a light emitting device, and an organic EL element of the present invention (Organic EL element 101 in a light emitting device) is covered with glass cover 102 (incidentally, sealing by the glass cover was carried out in a globe box under nitrogen ambience (under an ambience of high purity nitrogen gas at a purity of at least 99.999%) so that Organic EL Element 101 was not brought into contact with atmosphere).

FIG. 6 is a cross-sectional view of a light emitting device. In FIG. 6, 105 represents a cathode, 106 represents an organic EL layer, and 107 represents a glass substrate fitted with a transparent electrode. Further, the interior of glass cover 102 is filled with nitrogen gas 108 and water catching agent 109 is provided.

By employing an organic EL element of the present invention, it was possible to obtain a light emitting device having improved emission efficiency.

### EXAMPLES

Hereafter, the present invention will be described specifically by referring to Examples, however, the present invention is not limited to them. In Examples, the term “parts” or “%” is used. Unless particularly mentioned, it represents “mass parts” or “mass %”. In addition, a volume % of a compound in each example is obtained from a specific gravity by measuring a produced layer thickness with a quartz oscillator microbalance method and by calculating a mass.

### [Production of Organic EL Element]

### <Preparation of Organic EL Element 1-1>

An anode was prepared by making patterning to a glass substrate of 100 mm×100 mm×1.1 mm (NA45, produced by NH Techno Glass Corp.) on which ITO (indium tin oxide) was formed with a thickness of 100 nm. Thereafter, the above transparent support substrate provided with the ITO transparent electrode was subjected to ultrasonic washing with isopropyl alcohol, followed by drying with desiccated nitrogen gas, and was subjected to UV ozone washing for 5 minutes.

On the transparent support substrate thus prepared was applied a 70% solution of poly(3,4-ethylenedioxythiphene)-polystyrene sulfonate (PEDOT/PSS, Baytron PAI4083, made by Bayer AG.) diluted with water by using a spin coating method at 3,000 rpm for 30 seconds to form a film and then it was dried at 200° C. for one hour. A first hole injection layer having a thickness of 20 nm was prepared.

The resulting transparent support substrate was fixed to a substrate holder of a commercial vacuum deposition apparatus. Separately, 200 mg of α-NPD was placed in a molybdenum resistance heating boat, 200 mg of host compound (Comparative compound 1) was placed in another molybdenum resistance heating boat, 200 mg of a dopant compound (D-37) was placed in another molybdenum resistance heating boat, and 200 mg of BCP (2,9-dimethyl-4,7-diphenyl-1,10-phenanthroline) was placed in another molybdenum resistance heating boat. The resulting boats were fitted in the vacuum deposition apparatus.

Subsequently, after reducing the pressure of a vacuum tank to 4×10⁻⁴ Pa, the aforesaid heating boat containing α-NPD was heated via application of electric current and deposition was made onto the aforesaid hole injection layer at a deposition rate of 0.1 nm/second, whereby it was produced a hole transport layer having a thickness of 30 nm.

Further, the aforesaid heating boats each respectively containing a host compound (Comparative compound 1) and a dopant compound (D-37) were heated via application of electric current and co-deposition was carried out onto the aforesaid hole transport layer at a respective deposition rate of 0.1 nm/second and 0.010 nm/second, whereby it was produced a light emitting layer having a thickness of 40 nm.

Further, the aforesaid heating boat containing BCP was heated via application of electric current and deposition was carried out onto the aforesaid light emitting layer at a deposition rate of 0.1 nm/second, whereby it was produced an electron transport layer having a thickness of 30 nm.

Subsequently, 0.5 nm thick lithium fluoride was vapor deposited as a cathode buffer layer, and then, 110 nm thick aluminum was vapor deposited to form a cathode, whereby Organic EL element 1-1 was prepared.

### <Preparation of Organic EL Elements 1-2 to 1-24>>

Organic EL elements 1-2 to 1-24 were prepared in the same manner as preparation of Organic EL element 1-1 except that a host compound in the light emitting layer was changed with the compounds described in the following Table 1.

Comparative compound 1 and Comparative compound 2 are compounds indicated below.

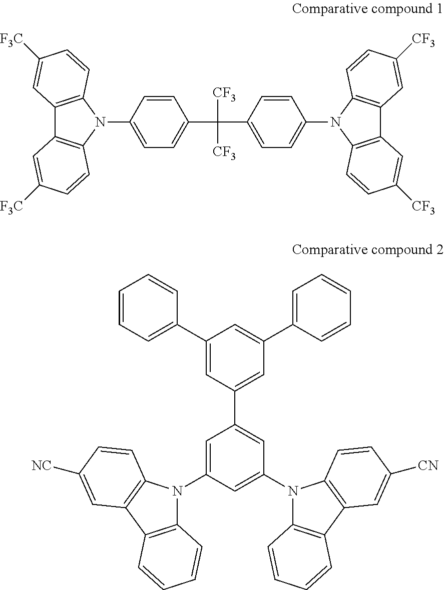 [SMILES: FC(F)(F)c1ccc2c(c1)c1cc(C(F)(F)F)ccc1n2-c1ccc(C(c2ccc(-n3c4ccc(C(F)(F)F)cc4c4cc(C(F)(F)F)ccc43)cc2)(C(F)(F)F)C(F)(F)F)cc1 ; N#Cc1ccc2c(c1)c1ccccc1n2-c1cc(-c2cc(-c3ccccc3)cc(-c3ccccc3)c2)cc(-n2c3ccccc3c3cc(C#N)ccc32)c1]

### [Evaluation]

### <Initial Driving Voltage>

Each organic EL element was driven at a constant electric current condition of 2.5 mA/cm2 at room temperature (about 23° C.). The voltage of the organic EL elements each was measured. The measured results were indicated as a relative value when the voltage of the organic EL element 1-1 was set to be 100.

Voltage=(Driving voltage of each organic EL element)/Driving voltage of organic EL element 1-1)×100

When this voltage value is smaller, it indicates that the driving voltage is smaller.
<Measurement of Change Rate of Resistance in the Light Emitting Layer Before and after Driving the Organic EL Element with Impedance Spectrometry>

By referring to the description in pp. 423 to 425 of “Handbook of Thin film evaluation” (published by Techno System, Co. Ltd.) and by using a 1260 type impedance analyzer with a 1296 type dielectric interface (made by Solartronanalytical Co.), the resistance value of the light emitting layer of the prepared organic EL element was measured.

Each organic EL element was driven with a constant electric current of 2.5 mA/cm² at a room temperature (25° C.) for 1,000 hours. The resistance values of the light emitting layer of each organic EL element were measured at the moment of before and after driving. The change rate of resistance was obtained according to the following calculating formula. In Table 1, the results were described as a relative value when the change rate of resistance for the organic EL element 1-1 was set to be 100.

Change rate of resistance before and after driving=[(Resistance after driving/Resistance before driving)−1]×100

The case showing nearer to zero indicates that the change rate of before and after driving is smaller. That is, it indicates that the voltage increase during driving is small.

### <Emission Efficiency>

Each organic EL element was lighted with a constant electric current of 2.5 mA/cm² at a room temperature (about 23° C.). The luminance [cd/m²] immediately after lighting was measured, and an external extraction quantum efficiency (q) (emission efficiency) value was calculated. The measurement of luminance was done with a spectroradiometer CS-1000 (produced by Konica Minolta, Inc.). The external extraction quantum efficiency was indicated as a relative value when the external extraction quantum efficiency value for the organic EL element 1-1 was set to be 100.

TABLE 1

| Organic | | | | | |
|---|---|---|---|---|---|
| EL | | | Change | | |
| element | | Initial driving | rate of | Emission | |
| No. | Host compound | voltage | resistance | efficiency | Remarks |
| 1-1 | Comparative compound 1 | 100 | 100 | 100 | *1 |
| 1-2 | Comparative compound 2 | 98 | 110 | 95 | *1 |
| 1-3 | Exemplified compound 3 | 70 | 83 | 121 | *2 |
| 1-4 | Exemplified compound 13 | 66 | 79 | 125 | *2 |
| 1-5 | Exemplified compound 18 | 75 | 80 | 122 | *2 |
| 1-6 | Exemplified compound 20 | 83 | 85 | 115 | *2 |
| 1-7 | Exemplified compound 25 | 67 | 77 | 119 | *2 |
| 1-8 | Exemplified compound 27 | 73 | 81 | 121 | *2 |
| 1-9 | Exemplified compound 37 | 82 | 81 | 116 | *2 |
| 1-10 | Exemplified compound 38 | 77 | 78 | 128 | *2 |
| 1-11 | Exemplified compound 7 | 85 | 83 | 113 | *2 |
| 1-12 | Exemplified compound 28 | 83 | 80 | 117 | *2 |
| 1-13 | Exemplified compound 39 | 78 | 80 | 123 | *2 |
| 1-14 | Exemplified compound 23 | 82 | 83 | 120 | *2 |
| 1-15 | Exemplified compound 40 | 78 | 85 | 118 | *2 |
| 1-16 | Exemplified compound 43 | 79 | 84 | 120 | *2 |
| 1-17 | Exemplified compound 46 | 80 | 81 | 121 | *2 |
| 1-18 | Exemplified compound 49 | 83 | 83 | 115 | *2 |
| 1-19 | Exemplified compound 50 | 78 | 79 | 119 | *2 |
| 1-20 | Exemplified compound 60 | 80 | 80 | 120 | *2 |
| 1-21 | Exemplified compound 62 | 78 | 81 | 119 | *2 |
| 1-22 | Exemplified compound 63 | 79 | 80 | 122 | *2 |
| 1-23 | Exemplified compound 64 | 83 | 82 | 119 | *2 |
| 1-24 | Exemplified compound 65 | 81 | 80 | 120 | *2 |

*1: Comparative example
*2: Present invention

As clearly indicated by the results listed in Table 1, the organic EL elements of the present invention are recognized to have a small amount of decrease in the initial driving voltage, and a small amount of change in resistance before driving and after driving, namely, a small amount of voltage increase during driving compared with a comparative organic EL element. Further, the organic EL elements of the present invention are excellent in emission efficiency.

### Example 2

Organic EL elements 2-1 to 2-15 were produced in the same manner as production of the organic EL element 1-1 except that the dopant D-37 was replaced with a dopant D-36, and the host compound was replaced with compounds described in Table 2.

TABLE 2

| | | Initial | Change rate | | | |
|---|---|---|---|---|---|---|
| Organic EL | | driving | of | Emission | Exciton | |
| element No. | Host compound | voltage | resistance | efficiency | stability | Remarks |
| 2-1 | Comparative | 100 | 100 | 100 | 100 | *1 |
| | compound 1 | | | | | |
| 2-2 | Comparative | 96 | 108 | 93 | 98 | *1 |
| | compound 2 | | | | | |
| 2-3 | Exemplified | 60 | 70 | 104 | 130 | *2 |
| | compound 66 | | | | | |
| 2-4 | Exemplified | 59 | 69 | 103 | 137 | *2 |
| | compound 72 | | | | | |
| 2-5 | Exemplified | 61 | 71 | 105 | 128 | *2 |
| | compound 74 | | | | | |
| 2-6 | Exemplified | 57 | 65 | 103 | 138 | *2 |
| | compound 77 | | | | | |
| 2-7 | Exemplified | 57 | 67 | 104 | 135 | *2 |
| | compound 79 | | | | | |
| 2-8 | Exemplified | 58 | 68 | 103 | 136 | *2 |
| | compound 83 | | | | | |
| 2-9 | Exemplified | 59 | 67 | 105 | 139 | *2 |
| | compound 84 | | | | | |
| 2-10 | Exemplified | 57 | 65 | 105 | 142 | *2 |
| | compound 87 | | | | | |
| 2-11 | Exemplified | 56 | 66 | 104 | 140 | *2 |
| | compound 88 | | | | | |
| 2-12 | Exemplified | 60 | 70 | 104 | 141 | *2 |
| | compound 92 | | | | | |
| 2-13 | Exemplified | 60 | 68 | 103 | 139 | *2 |
| | compound 99 | | | | | |
| 2-14 | Exemplified | 59 | 67 | 102 | 140 | *2 |
| | compound 105 | | | | | |
| 2-15 | Exemplified | 60 | 70 | 103 | 138 | *2 |
| | compound 110 | | | | | |

*1: Comparative example
*2: Present invention

### [Evaluation]

### <Initial Driving Voltage, Change Rate of Resistance, and Emission Efficiency>

Initial driving voltage, change rate of resistance (change rate of resistance in the light emitting layer with Impedance spectrometry, and Emission efficiency), and emission efficiency were measured in the same manner as done in Example 1. The evaluation results were represented as a relative value when the value of the organic EL element 2-1 was set to be 100.

### <Exciton Stability>

A host compound and a dopant (D-36) were deposited on a quartz substrate (with respective deposition rate of 0.1 nm/sec and 0.010 nm/sec) to obtain a co-deposition film. The non-light emitting surface was covered with a glass case, and a 300 μm thick glass substrate was employed as a sealing substrate. An epoxy based light curable type adhesive (LUXTRACK LC0629B produced by Toagosei Co., Ltd.) was employed in the periphery as a sealing material. The resulting one was superimposed on the cathode to be brought into close contact with the transparent support substrate, and curing and sealing were carried out via exposure of UV radiation onto the glass substrate side. This single light emitting layer was irradiated with UV-LED (5 W/cm²) for 20 minutes. The distance between the lighting source and the sample was made to be 15 mm. A constant current of 2.5 mA/cm² was applied to the sample irradiated with UV rays, and emission luminance immediately after lighting was measured. The residual rate of luminance was calculated based on the following scheme. Here, the initial emission luminance (LO) is an emission luminance at the time of evaluation of emission efficiency.

Exciton stability (%)=(Emission luminance after irradiation with UV for 20 minutes)/(Initial emission luminance (LO))×100

In Table 2, the evaluation results were indicated as a relative value when the value for the organic EL element 2-1 was set to be 100. When the value of the residual rate of luminance is larger, it indicates that it is excellent in exciton stability. The durability of the organic EL element of the present invention was revealed to be high compared with a comparative organic EL element.

### INDUSTRIAL APPLICABILITY

By the present invention, it is possible to obtain a specific material for an organic EL element. The material suppresses decrease of the initial driving voltage, and the voltage increase during driving of the organic EL element, and further it enables to improve emission efficiency. The material is suitably used for an organic EL element, an organic EL display, and a various kinds of indicating devices such as a touch panel equipped with the organic EL element.

### DESCRIPTION OF SYMBOLS

1: Display
3: Pixel
5: Scanning line
6: Data line
7: Electric source line
10: Organic EL element
11: Switching transistor
12: Operating transistor
13: Capacitor
101: Organic EL element in a light emitting device
102: Glass cover
105: Cathode
106: Organic layer
107: Glass substrate having a transparent electrode
108: Nitrogen gas
109: Water catching agent
A: Display section
B: Control section
C: Wiring section
L: Emission light

1. An organic electroluminescent element material containing a compound having a structure represented by Formula (1), 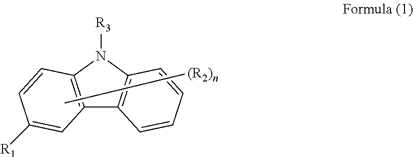
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; and n represents an integer of 0 to 7, provided that when R₂ and R₃ each independently represent an alkyl group, an aryl group, a heteroaryl group, or a fluoroalkyl group, at least one of R₂ and R₃ contains a structure represented by Formula (2), 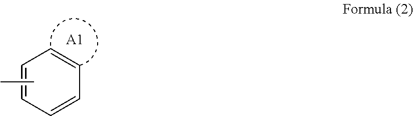
wherein A1 represents a 5-membered heterocycle, provided that the 5-membered heterocycle may further have a substituent, and the substituent may form a ring.

2. The organic electroluminescent element material described in claim 1,
wherein in the compound represented by Formula (1), when R₂ and R₃ each independently represent an alkyl group, an aryl group, a heteroaryl group, or a fluoroalkyl group, at least one of R₂ and R₃ contains a substituent represented by Formula (2).

3. The organic electroluminescent element material described in claim 1,
wherein R₂ and R₃ each independently represent an alkyl group, an aryl group, a heteroaryl group, or a fluoroalkyl group, at least one of R₂ and R₃ in itself represents a substituent represented by Formula (2).

4. The organic electroluminescent element material described in claim 1,
wherein the compound having a structure represented by Formula (1) is a compound having a structure represented by Formula (3), 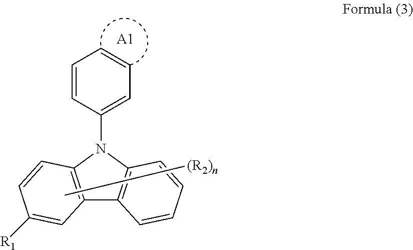
wherein R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and A1 represents a 5-membered heterocycle, provided that the 5-membered heterocycle may further have a substituent, and the substituent may form a ring.

5. The organic electroluminescent element material described in claim 1,
wherein the compound having a structure represented by Formula (1) is a compound having a structure represented by Formula (4), 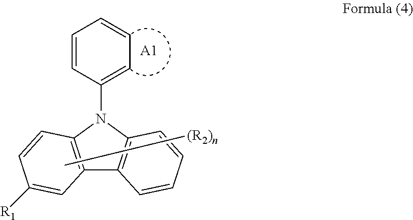
wherein R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and A1 represents a 5-membered heterocycle, provided that the 5-membered heterocycle may further have a substituent, and the substituent may form a ring.

6. The organic electroluminescent element material described in claim 1,
wherein the compound having a structure represented by Formula (1) is a compound having a structure represented by Formula (5), 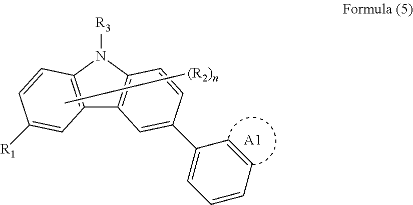
wherein R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; n represents an integer of 0 to 6; and A1 represents a 5-membered heterocycle, provided that the 5-membered heterocycle may further have a substituent, and the substituent may form a ring.

7. The organic electroluminescent element material described in claim 1,
wherein the compound having a structure represented by Formula (1) is a compound having a structure represented by Formula (6), 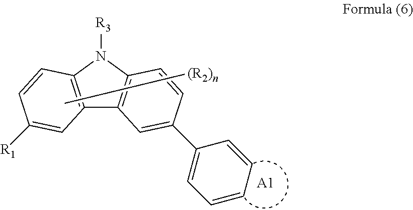
wherein R₁ represents a cyano group or CF₃; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; n represents an integer of 0 to 6; and A1 represents a 5-membered heterocycle, provided that the 5-membered heterocycle may further have a substituent, and the substituent may form a ring.

8. The organic electroluminescent element material described in claim 1,
wherein A1 in Formula (2) represents one selected from the group consisting of: a furan ring, a thiophene ring, a pyrrole ring, an indole ring, a benzofuran ring, a benzothiophene ring, a pyrazole ring, an imidazole ring, a triazole ring, an oxazole ring, and a thiazole ring.

9. The organic electroluminescent element material described in claim 1,
wherein the compound having a structure represented by Formula (1) has an emission maximum wavelength of a 0-0 transition band in a phosphorescence spectrum to be 450 nm or less.

10. The organic electroluminescent element material described in claim 1,
wherein a LUMO level of a condensed ring compound in a substituent containing a structure represented by Formula (2) is lower than a LUMO level of carbazole.

11. The organic electroluminescent element material described in claim 1,
wherein the compound having a structure represented by Formula (1) is a compound having a structure represented by Formula (7), 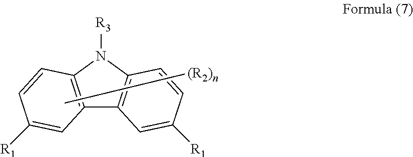
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; and n represents an integer of 0 to 6, provided that when R₂ and R₃ each independently represent an alkyl group, an aryl group, a heteroaryl group, or a fluoroalkyl group, at least one of R₂ and R₃ contains a structure represented by Formula (2).

12. The organic electroluminescent element material described in claim 1,
wherein the compound having a structure represented by Formula (1) is a compound having a structure represented by Formula (8), 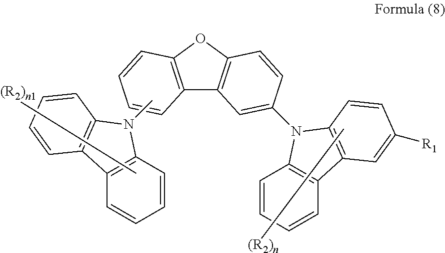
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

13. The organic electroluminescent element material described in claim 1,
wherein the compound having a structure represented by Formula (1) is a compound having a structure represented by Formula (9), 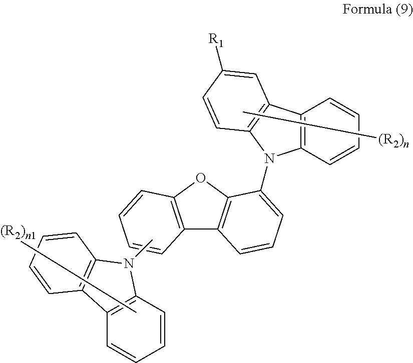
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

14. The organic electroluminescent element material described in claim 1,
wherein the compound having a structure represented by Formula (1) is a compound having a structure represented by Formula (10), 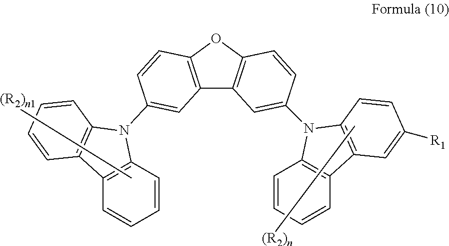
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

15. The organic electroluminescent element material described in claim 1,
wherein the compound having a structure represented by Formula (1) is a compound having a structure represented by Formula (11), 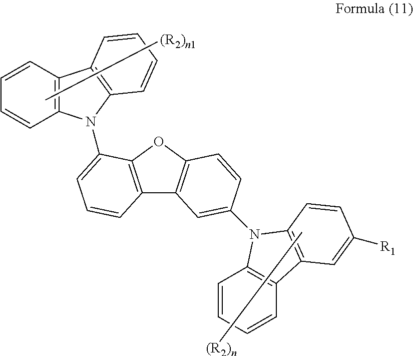
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

16. The organic electroluminescent element material described in claim 1,
wherein the compound having a structure represented by Formula (1) is a compound having a structure represented by Formula (12), 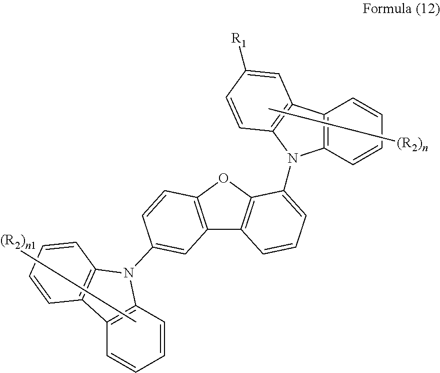
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

17. The organic electroluminescent element material described in claim 1,
wherein the compound having a structure represented by Formula (1) is a compound having a structure represented by Formula (13), 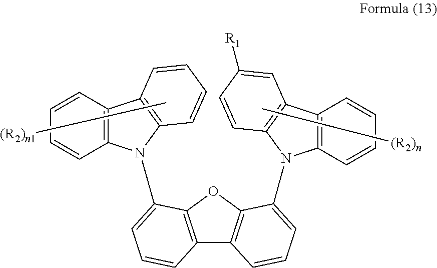
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 8.

18. The organic electroluminescent element material described in claim 1,
wherein the compound having a structure represented by Formula (1) is a compound having a structure represented by Formula (14), 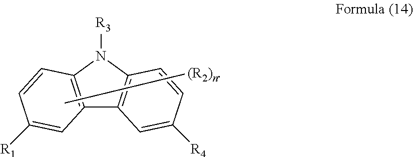
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; R₃ represents a hydrogen atom, an alkyl group, an aryl group, a heteroaryl group, or a fluoroalkyl group; R₄ represents a dibenzofuran ring; and n represents an integer of 0 to 6, provided that when R₂ and R₃ each independently represent an alkyl group, an aryl group, a heteroaryl group, or a fluoroalkyl group, at least one of R₂ and R₃ contains a structure represented by Formula (2).

19. The organic electroluminescent element material described in claim 1,
wherein the compound having a structure represented by Formula (1) is a compound having a structure represented by Formula (15), 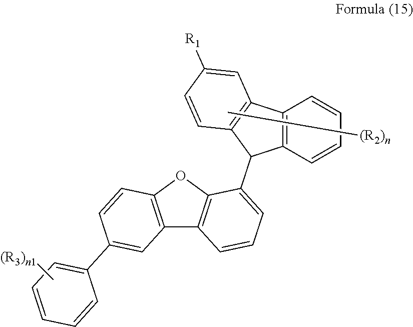
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 5.

20. The organic electroluminescent element material described in claim 1,
wherein the compound having a structure represented by Formula (1) is a compound having a structure represented by Formula (16), 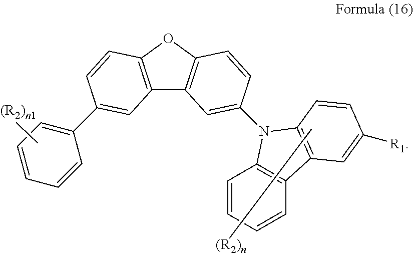
wherein R₁ represents a cyano group, CₘF₂ₘ₊₁, or SF₅; m represents an integer of 1 to 18; R₂ represents an alkyl group, an aryl group, a heteroaryl group, a halogen atom, a cyano group, or a fluoroalkyl group that is substituted with any one of hydrogen atoms on carbon atoms constituting a carbazole ring; n represents an integer of 0 to 7; and n1 represents an integer of 0 to 5.

21. An organic electroluminescent element containing the organic electroluminescent element material described in claim 1.

22. The organic electroluminescent element described in claim 21 emitting blue light.

23. The organic electroluminescent element described in claim 21 emitting white light.

24. A display device equipped with the organic electroluminescent element described in claim 21.

25. A lighting device equipped with the organic electroluminescent element described in claim 21.
